(19) United States
(12) Patent Application Publication
(10) Pub. No.: US 2025/0332284 A1
(43) Pub. Date: Oct. 30, 2025

(54) MODIFIED PLANT VIRUS SYSTEM FOR DELIVERY OF NUCLEIC ACIDS INTO MAMMALIAN CELLS

(71) Applicant: GEORGIA TECH RESEARCH CORPORATION, Atlanta, GA (US)

(72) Inventors: James Dahlman, Atlanta, GA (US); Curtis Dobrowolski, Boston, MA (US); Kalina Paunovska, Atlanta, GA (US)

(21) Appl. No.: 18/841,225

(22) PCT Filed: Feb. 24, 2023
(86) PCT No.: PCT/US2023/063239

§ 371 Date: Aug. 23, 2024

Related U.S. Application Data
(60) Provisional application No. 63/314,167, filed on Feb. 25, 2022.

(51) Int. Cl. A61K 48/00; C07K 14/005; C12N 9/22; C12N 15/11; C12N 15/86

(52) U.S. Cl. CPC A61K 48/005; C07K 14/005; C12N 9/222; C12N 15/11; C12N 15/86; C12N 2310/20; C12N 2740/00022; C12N 2740/00043; C12N 2750/12022; C12N 2750/12043; C12N 2770/40022; C12N 2770/40043

(57) ABSTRACT

The disclosure provides modified plant viruses designed for delivering a nucleotide of interest into mammalian cells. The modified plant viruses include a plant virus nucleotide sequence (e.g. fragment) that is capable of transfecting a mammalian cell when that mammalian cell expresses a receptor for the modified plant virus. Accordingly, the disclosure also provides receptors for the modified plant viruses as well as methods of using the receptor or the modified plant virus and receptor.

### CROSS-REFERENCE TO RELATED APPLICATIONS

The present application is the U.S. national stage entry of PCT/US2023/063239, filed Feb. 24, 2023, which claims the benefit of priority to U.S. Provisional Application No. 63/314,167, filed Feb. 25, 2022, the entire contents of both of which are incorporated herein by reference.

### SEQUENCE LISTING

The instant application contains a Sequence Listing which has been submitted electronically in XML format and is hereby incorporated by reference in its entirety. Said XML copy, created on Jun. 29, 2024, is named 119917000017_Sequence Listing and is 892 kilobytes size.

### TECHNICAL FIELD

This disclosure relates to modified plant virus systems that designed for delivery of nucleic acids into a mammalian cell.

### BACKGROUND

Delivery of nucleic acids into mammalian cells can be achieved by a variety of ways, including viral methods and non-viral methods.

One common way to currently deliver nucleic acids into cells involves adenoviral vectors. Adenoviral vectors are able to be used for delivery for a variety of cell types within and outside of the immune system. However, their use is limited because they are only suitable for delivery of nucleic acids of a specific size, and they are unable to specifically target a unique cell of interest necessitating re-optimization of the vector for new cell types. What is needed is a viral based delivery system for nucleic acids into mammalian cells.

### SUMMARY

The disclosure provides for modified plant viruses designed for delivering a nucleotide of interest into mammalian cells. The modified plant viruses include a plant virus nucleotide sequence (e.g. fragment) that is capable of transfecting a mammalian cell when that mammalian cell expresses a receptor for the modified plant virus. Accordingly, the disclosure also provides receptors for the modified plant viruses as well as methods of using the receptor or the modified plant viruses and receptor.

One embodiment of the invention is a modified plant virus comprising a plant virus nucleotide sequence and a nucleotide sequence capable of exhibiting a therapeutic effect in a mammalian cell, wherein the virus is capable of transfecting a mammalian cell that has been modified to express receptor for the modified plant virus. The mammalian cell can express the nucleotide sequence capable of exhibiting a therapeutic effect upon transfection. The nucleotide sequence capable of exhibiting a therapeutic effect can be a mammalian gene or a nuclease.

In certain embodiments, the plant virus nucleotide sequence is obtained from Cauliflower Mosaic virus, Tomato Yellow Leaf Curl Virus (TYLCV) or Turnip Yellow Virus (TuYV). For example, the plant virus nucleotide sequence may be obtained from SEQ ID NO: 1-6.

The disclosure also provides for modified receptor for the modified plant virus. In certain embodiments, the modified receptor are the receptors for Cauliflower Mosaic virus, Tomato Yellow Leaf Curl Virus (TYLCV) or Turnip Yellow Virus (TuYV) or a functional fragment thereof. In certain embodiments, the modified receptor for the modified plant virus comprises the amino acid sequence of SEQ ID NO: 7, 10, or 13 or a functional fragment thereof, wherein the modified receptor or functional fragment thereof is capable of rendering a mammalian cell susceptible to infection with a plant virus or a derivative thereof. In some embodiments, the fragment comprises a peptide having at least 85%, 90%, 95%, 96%, 97%, 98% or 99% identity to amino acids 103 to 504 of SEQ ID NO: 4. SEQ ID NO 10 or SEQ ID NO: 13, wherein the fragment is capable of rendering a mammalian cell susceptible to infection with the modified plant virus.

Alternatively, modified receptor is encoded by the nucleotide sequence of SEQ ID NO: 8, 9, 11, 12, 14, or 15, or a functional fragment thereof (such as e.g. the protein encoded by a nucleotide having at least 85%, 90%, 95%, 96%, 97%, 98% or 99% identity to SEQ ID NO: 8, 9, 11, 12, 14, or 15), wherein the modified receptor or functional fragment thereof is capable of rendering a mammalian cell susceptible to infection with plant virus or a derivative thereof. In certain embodiments, the disclosure provides for compositions comprising the modified receptor, wherein the composition is formulated for delivery to a specific cell type.

In another embodiment, the disclosure is directed to a method of transiently expressing a receptor for a modified plant virus in a mammalian cell which includes delivering a composition containing the modified receptor for the modified plant virus into the mammalian cell, whereby the mammalian cell transiently expresses the modified receptor for the modified plant virus upon delivery of the modified receptor for the modified plant virus nucleotide.

In another embodiment, the discloses provides for a method of delivering a nucleotide sequence capable of exhibiting a therapeutic effect in a mammalian cell to a mammalian cell transiently expressing the receptor for the modified plant virus. In certain embodiments, the method includes contacting the mammalian cell with a modified plant virus comprising a plant virus nucleotide sequence and a nucleotide sequence capable of exhibiting a therapeutic effect in a mammalian cell, whereby the virus is capable of transfecting a mammalian cell that has been modified to express a receptor for the modified plant virus and wherein the mammalian cell expresses the nucleotide sequence capable of exhibiting a therapeutic effect upon transfection.

### BRIEF DESCRIPTION OF THE DRAWINGS

The file of this patent or application contains at least one drawing/photograph executed in color. Copies of this patent or patent application publication with color drawing(s)/photograph(s) will be provided by the Office upon request and payment of the necessary fee.

The summary, as well as the following detailed description, is further understood when read in conjunction with the appended drawings. For the purpose of illustrating the invention, there are shown in the drawings' exemplary embodiments of the invention. However, the invention is not limited to the specific methods and compositions disclosed and the invention is not limited to the precise arrangements and instrumentalities of the embodiments depicted in the drawings. In addition, the drawings are not necessarily drawn to scale. In the drawings:

FIG. 1 shows a schematic of a general method of using plant viruses to deliver a nucleic acid to a mammalian cell.

FIG. 2A and FIG. 2B show the effect of administering a plant virus to a mammalian cell that has not been treated with the receptor (FIG. 2A) and to a mammalian cell that has been treated with the receptor (FIG. 2B).

FIG. 3A-3D show delivery of mRNA encoding viral receptor and successful delivery using LNP-liver in humanized mice.

FIG. 4A-4H show the quantification of CaMV levels using protein methods.

FIG. 5 shows quantification of CaMV levels using digital droplet PCR (ddPCR). CaMV RNA levels can be detected 48 hours after administration with CaMV in mice pre-treated with Stylin mRNA.

FIG. 6A-6D establish that it is possible to generate a plant cell (BY-2 cells) capable of producing CaMV encoding for GFP.

FIG. 7 shows single cell RNA sequencing data for humanized mouse experiment. Mice were treated with LNPs containing aVHH+Stylin or Stylin alone 24 hours prior to treatment with CaMV. In mice treated with aVHH and Stylin mRNA CaMV RNA was observed (shown in purple); in mice treated with either Stylin only or PBS only, no CaMV RNA was observed.

FIG. 8A-8G show the results of checkerboard experiments to identify genes that are most important to produce functional CAMV.

### DETAILED DESCRIPTION

The general description and the following detailed description are exemplary and explanatory only and are not restrictive of the invention, as defined in the appended claims. Other aspects of the present invention will be apparent to those skilled in the art in view of the detailed description of the invention as provided herein.

This disclosure is directed to modified plant virus which are modified to carry a nucleic acid of interest and receptors for these modified plant viruses. The modified plant virus does not enter mammalian cells on its own. This disclosure is based on the discovery that through transient expression of the receptor for the modified plant virus in a mammalian cell that is normally not susceptible to infection with the virus it is possible to specifically target delivery of the modified plant virus containing the nucleic acid of interest to only cells expressing the receptor. After delivery of the nucleic acid of interest, the nucleic acid is expressed in the cells. Furthermore, this disclosure is based on the discovery that it is possible to specifically target the receptors to certain cells of interest. Accordingly, it is possible to provide cell specific gene therapy.

Accordingly this disclosure provides for more specific gene therapies than those currently viable. Since the modified plant viruses of the disclosure do not enter mammalian (e.g. human) cells without its receptor (expression of which controlled), without being pound by theory, the disclose provides for reduced viral delivery to ‘off-target’ cells (i.e., mammalian (human) cells without the receptor).

The disclosure also provides for less expensive gene therapy. Currently, the cost to manufacture enough AAV for a single human injection can be as high as $400 k. As a result, AAV companies cannot reduce pricing to levels that would render AAV therapy widely used. Furthermore, unlike AAV therapy, which requires re-optimization of the AAV for a new cell type, by controlling delivery of the receptor to a specific cell type, the modified plant viruses of the disclosure may be used with a variety of different mammalian cell types without the need of re-optimization for a new cell type.

### A. Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or ±10%, more preferably ±5%, even more preferably +1%, and still more preferably ±0.1% from the specified value, as such variations are appropriate to perform the disclosed methods.

The term “biological” or “biological sample” refers to a sample obtained from an organism or from components (e.g., cells) of an organism. The sample may be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

As used herein, the terms “control,” or “reference” can be used interchangeably and refer to a value that is used as a standard of comparison.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “RNA” as used herein is defined as ribonucleic acid.

The term “treatment” as used within the context of the present invention is meant to include therapeutic treatment as well as prophylactic, or suppressive measures for the disease or disorder. As used herein, the term “treatment” and associated terms such as “treat” and “treating” means the reduction of the progression, severity and/or duration of a disease condition or at least one symptom thereof. The term “treatment” therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids. As used herein, when a nucleic acid sequenced is provided as a DNA sequence, it should be understood that the RNA sequence may also be used.

Nucleic acids can be single stranded or double-stranded or can contain portions of both double-stranded and single-stranded sequence. The nucleic acid can be DNA, both genomic and cDNA, RNA, or a hybrid, where the nucleic acid can contain combinations of deoxyribo and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods. “Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

“Substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more amino acids. Substantially identical can also mean that a first nucleic acid sequence and a second nucleic acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means Watson-Crick (e.g., A-T/U and CG) or Hoogsteen base pairing between nucleotides or nucleotide analogs of nucleic acid molecules.

“Consensus” or “Consensus Sequence” as used herein may mean a synthetic nucleic acid sequence, or corresponding polypeptide sequence, constructed based on analysis of an alignment of multiple subtypes of a particular antigen. The sequence may be used to induce broad immunity against multiple subtypes, serotypes, or strains of a particular antigen. Synthetic antigens, such as fusion proteins, may be manipulated to generate consensus sequences (or consensus antigens).

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. Identity can be performed manually or by using a computer sequence algorithm such as BLAST or BLAST 2.0.

“Variant” used herein with respect to a nucleic acid means (i) a portion or fragment of a referenced nucleotide sequence: (ii) the complement of a referenced nucleotide sequence or portion thereof: (iii) a nucleic acid that is substantially identical to a referenced nucleic acid or the complement thereof: or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein means a nucleic acid sequence containing an origin of replication. A vector can be a viral vector, bacteriophage, bacterial artificial chromosome, or yeast artificial chromosome. A vector can be a DNA or RNA vector. A vector can be a self-replicating extrachromosomal vector, and preferably, is a DNA plasmid.

As used herein, the term “a modified receptor for the modified plant virus nucleotide” refers to a nucleotide encoding a modified receptor for the modified plant virus of the disclosure. The nucleotide may be RNA or DNA.

As used herein, the term “pharmaceutical composition” refers to a mixture of at least one compound useful within the invention with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to intra-tumoral, intravenous, intrapleural, oral, aerosol, parenteral, ophthalmic, pulmonary, and topical administration.

The language “pharmaceutically acceptable carrier” includes a pharmaceutically acceptable salt, pharmaceutically acceptable material, composition, or carrier, such as a liquid or solid filler, diluent, excipient, solvent, or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soy bean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Modified Plant Viruses for Delivery of Nucleic Acids Into Mammalian Cells

One aspect of the disclosure is related to modified plant virus that has been modified to deliver a nucleic acid into a mammalian cell which has been modified to express a receptor for the modified plant virus.

Accordingly, one embodiment of the invention is a modified plant virus comprising a plant virus nucleotide sequence and a nucleotide sequence to be expressed in a mammalian cell. The modified virus is capable of transfecting a mammalian cell that has been modified to express a receptor for the modified plant virus and the mammalian cell expresses the nucleic acid upon transfection.

Suitable plant virus nucleotide sequences include but are not limited to variants or fragments of plant viruses whereby the variants or fragments retain the plant virus's ability to transfect cells when the appropriate receptor is present on the cell.

In certain embodiments, the nucleotide sequence is a nucleotide sequence capable of exhibiting a therapeutic effect in a mammalian cell, such as for example a mammalian gene. In other embodiments, the nucleotide sequence capable of exhibiting a therapeutic effect is a nuclease. Examples of such nucleases include but are not limited to a CRISPR/cas construct, TALENS, or ZFNs.

In certain embodiments, the modified plant virus sequence nucleotide sequence comprises one or more of ORF I, ORF II, ORF III, ORF IV, ORF V, ORF VI, ORF VII and ORF VIII of the plant virus. In certain embodiments, the plant virus nucleotide sequence is obtained from Cauliflower Mosaic virus. Tomato Yellow Leaf Curl Virus (TYLCV) or Turnip Yellow Virus (TuYV).

In certain embodiments, the nucleotide sequence (such as the nucleotide sequence capable of exhibiting a therapeutic effect in a mammalian cell) has been inserted into the plant virus nucleotide sequence. In other embodiments, the modified plant virus comprises a first plant virus sequence and a second plant virus sequence and wherein the nucleotide sequence (such as the nucleotide sequence capable of exhibiting a therapeutic effect in a mammalian cell) has been inserted the second plant virus nucleotide sequence.

In one embodiment of the modified plant, the plant virus nucleotide sequence comprises the nucleotide sequence of SEQ ID NO: 1-6 or a fragment thereof capable of transfecting a mammalian cell that has been modified to express a receptor for the modified plant virus.

In certain embodiments, the nucleotide sequence (such as the nucleotide capable of exhibiting a therapeutic effect in a mammalian cell) has been inserted in the plant virus sequence of SEQ ID NO: 1-3, 5 or 6. For example, the nucleotide (such as the nucleotide capable of exhibiting a therapeutic effect in a mammalian cell) has been inserted in the plant virus sequence of SEQ ID NO: 2 at nucleotides 3424-6101. Alternatively, the nucleotide (such as the nucleotide capable of exhibiting a therapeutic effect in a mammalian cell) has been inserted in the plant virus sequence of SEQ ID NO: 3 at nucleotides 1596-6101. In yet another embodiment, the nucleotide (such as the nucleotide capable of exhibiting a therapeutic effect in a mammalian cell) has been inserted in the plant virus sequence of SEQ ID NO: 5 at position 875. In yet an alternate embodiment, the nucleotide sequence capable of exhibiting a therapeutic effect in a mammalian cell has been inserted in the plant virus sequence of SEQ ID NO: 6 at position 4946.

In certain embodiments, the modified plant virus is based on Cauliflower Mosaic Virus. In one embodiment, the modified Cauliflower Mosaic Virus comprises SEQ ID NO: 1 or a functional fragment thereof which is capable of infecting a cells. In another embodiment, the modified Cauliflower Mosaic Virus comprises SEQ ID NO: 1 or a functional fragment thereof which is capable of infecting a cells. In an alternate embodiment, the modified Cauliflower Mosaic Virus comprises SEQ ID NO: 1 or a functional fragment thereof which is capable of infecting a cells.

In other embodiments, the modified plant virus is based on Tomato Yellow Leaf Curl Virus (TYLCV). Tomato Yellow Leaf Curl Virus (TYLCV) is a two-component virus—one fragment encodes the genome of TYCLV and the other encodes the transgene fragment. In one embodiment, the modified TYCLV comprises SEQ ID NO: 4 and SEQ ID NO: 5 or a functional fragment thereof which is capable of infecting cells.

The disclosure encompasses any part of the nucleotide sequence from Cauliflower Mosaic virus (CaMV), Tomato Yellow Leaf Curl Virus (TYLCV) or Turnip Yellow Virus (TuYV) (SEQ ID NO: 1-6) that are capable of transfecting a mammalian cell that has been modified to express a receptor for the modified plant virus (CaMV, TYLCV, or TuYV).

The disclosure also includes plant cells containing the modified plant viruses.

In addition the disclosure also includes methods of generating the modified plant virus. In certain embodiments, the methods include providing the plant virus, such as CaMV, TYLCV, or TuYV, or a fragment thereof and inserting the nucleotide of interest (e.g. the nucleotide capable of exhibiting a therapeutic effect in a mammalian cell) into the plant virus or fragment thereof. The result modified plant virus is capable of transfecting a mammalian cell that has been modified to express a receptor for the modified plant virus. In certain embodiments, the method includes inserting the nucleotide into any one of SEQ ID NO: 1-6 or a fragment thereof capable of transfecting a mammalian cell that has been modified to express a receptor for the modified plant virus. The method may include inserting the nucleotide of interest at nucleotides 3424-6101 of SEQ ID NO: 2, at nucleotides 1596-6101 of SEQ ID NO: 3, at position 875 of SEQ ID NO: 4, or at position 4946 of SEQ ID NO: 6.

### C. Modified Plant Receptors for the Modified Plant Viruses

Another aspect of the disclosure is directed to a modified receptor for the modified plant virus, wherein the modified receptor or functional fragment thereof is capable of rendering a mammalian cell susceptible to infection with a plant virus, variant, fragment, or a derivative thereof.

The modified plant receptor for use in the instant disclosure contain the receptor domain of a receptor for the plant virus or any fragment thereof capable of binding the plant virus. The modified plant virus receptors have a membrane anchor (such as e.g. a GPI anchor) and optionally a signal peptide.

In certain embodiments, the modified receptors are the receptors for Cauliflower Mosaic virus, Tomato Yellow Leaf Curl Virus (TYLCV), Turnip Yellow Virus (TuYV), or a functional fragment thereof. For example, the receptor may be Stylin-1 (CaMV), BtPGRP receptor (TYLCV), mpEPH receptor (TuYV), or a fragment thereof capable of binding the modified virus. In certain embodiments, the modified receptors include a signal peptide (such as CD55) as well as a membrane anchor (such as CD55 GPI anchor).

Suitable modified receptors include receptors or functional fragments thereof that retain that ability of the plant virus binding so that the modified receptors can for selective transfection of mammalian cells with the modified plant virus.

In one embodiment, the modified receptor for a modified plant virus comprises the amino acid sequence of SEQ ID NO: 7, 10, or 13 or a functional fragment thereof capable of rendering a mammalian cell susceptible to infection with a plant virus or a derivative thereof. In an embodiment, the modified receptor protein comprises an amino acid sequence at least 85%, 86%, 87%, 88%, 89%, 90%, 81%, 92%, 93%, 94%, 95%, 96%, 97%, 98% or 99% identical to SEQ ID NO: 7, 10, or 13.

In certain embodiment, the modified receptor fragment includes an extracellular domain of the receptor. For example, the fragment comprises amino acids 103 to 504 of SEQ ID NO: 4. Alternative, the fragment comprises a peptide having at least 885%, 86%, 87%, 88%, 89%, 90%, 81%, 92%, 93%, 94%, 95%, 96%, 97%, 98% or 99% identity to amino acids 103 to 504 of SEQ ID NO: 4 and capable of rendering a mammalian cell susceptible to infection with the modified plant virus.

In other embodiments, the fragment comprises amino acids 35-268 of SEQ ID NO: 10. Alternatively, the fragment comprises a peptide having at least 85%, 86%, 87%, 88%, 89%, 90%, 81%, 92%, 93%, 94%, 95%, 96%, 97%, 98% or 99% identity to amino acids 35-268 of SEQ ID NO: 10 and capable of rendering a mammalian cell susceptible to infection with the modified plant virus.

In yet another embodiment, the fragment comprises amino acids 35-653 of SEQ ID NO: 13. In alternate embodiments, the fragment comprises a peptide having at least 85%, 86%, 87%, 88%, 89%, 90%, 81%, 92%, 93%, 94%, 95%, 96%, 97%, 98% or 99% identity to amino acids 35-653 of SEQ ID NO: 13 and capable of rendering a mammalian cell susceptible to infection with the modified plant virus.

In one embodiment, the fragment also includes GPI anchor and/or a signal peptide. In another embodiment, the fragment includes a GPI anchor and a signal peptide.

In certain aspects of the disclosure, the modified receptor for the modified plant virus, consists of the amino acid sequence of SEQ ID NO: 7, 10, or 13, or a functional fragment thereof, and is capable of rendering a mammalian cell susceptible to infection with plant virus or a derivative thereof.

In alternate embodiments, the modified receptor for the modified plant virus is encoded by the nucleotide sequence of SEQ ID NO: 8, 9, 11, 12, 14, or 15, by a nucleotide having at least 85%, 86%, 87%, 88%, 89%, 90%, 81%, 92%, 93%, 94%, 95%, 96%, 97%, 98% or 99% identity to SEQ ID NO: 8, 9, 11, 12, 14, or 15 or by or a functional fragment thereof, whereby the modified receptor or functional fragment thereof are capable of rendering a mammalian cell susceptible to infection with plant virus or a derivative thereof.

To transiently express the modified receptor for the modified plant virus. nucleic acids (nucleotides) encoding the modified receptor for the modified plant virus are used. Accordingly, one aspect of the disclosure is directed to nucleotides encoding the modified receptor for the modified plant virus (“a modified receptor for the modified plant virus nucleotide”). The nucleotide may be RNA or DNA. In certain embodiments, when the modified receptor for the modified plant virus nucleotide is used for transient expression of the modified receptor for the modified plant virus in cells, the nucleotide is RNA.

The disclosure is also directed to modified receptors for the modified plant virus nucleotide comprising the nucleotide sequence of SEQ ID NO: 8, 9, 11, 12, 14, or 15, by a nucleotide having at least 85%, 86%, 87%, 88%, 89%, 90%, 81%, 92%, 93%, 94%, 95%, 96%, 97%, 98% or 99% identity to SEQ ID NO: 8, 9, 11, 12, 14, or 15 or by or a functional fragment thereof, whereby the modified receptor or functional fragment thereof are capable of rendering a mammalian cell susceptible to infection with plant virus or a derivative thereof. In certain embodiments, the modified receptor for the modified plant virus nucleotide is selected from the group consisting of: (a) a modified receptor for the modified plant virus nucleotide comprising the nucleotide sequence of SEQ ID NO: 9, 12, or 15; (b) a modified receptor for the modified plant virus nucleotide consisting of the nucleotide sequence of SEQ ID NO: 9, 12, or 15; (c) a modified receptor for the modified plant virus nucleotide comprising a functional fragment of the nucleotide sequence of SEQ ID NO: 9, 12, or 15, wherein the functional fragment is capable of rendering a mammalian cell susceptible to infection with plant virus or a derivative thereof; and (d) combinations thereof. In certain embodiments, the nucleotide encoding the modified receptor is a codon optimized for human cells.

The disclosure also provides for cells or vectors containing the modified plant virus nucleotide.

In certain embodiments, the disclosure provides for compositions comprising the modified receptor, wherein the composition is formulated for delivery to a specific cell type. For example, the modified receptor may be in lipid nanoparticle that is specifically formulated for delivery into a certain cell type such as e.g. the liver.

### D. Pharmaceutical Compositions

The disclosure also provides for pharmaceutical compositions containing the modified plant virus and/or the modified receptor for the modified plant virus and a pharmaceutically acceptable carrier. In one embodiment, the pharmaceutical composition comprises the modified plant virus. In another embodiment, the pharmaceutical composition comprises the modified receptor.

In one embodiment, the disclosure is directed to a pharmaceutical composition comprising the modified receptor for the modified plant virus nucleotide and a pharmaceutically acceptable carrier. In certain embodiments, the composition comprises a lipid nanoparticle. Preferably, the composition is formulated for delivering the modified receptor for the modified plant virus nucleotide into mammalian cells.

Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single-or multi-dose unit.

The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs. In one embodiment, the subject is a human or a non-human mammal such as but not limited to an equine, an ovine, a bovine, a porcine, a canine, a feline and a murine. In one embodiment, the subject is a human.

In one embodiment, the compositions are formulated using one or more pharmaceutically acceptable excipients or carriers. Pharmaceutically acceptable carriers, which are useful, include, but are not limited to, glycerol, water, saline, ethanol, and other pharmaceutically acceptable salt solutions such as phosphates and salts of organic acids. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

The compositions may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea, and combinations thereof.

The compositions may include an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

The pharmaceutical composition disclosed herein may be used in combination with an additional therapeutic agent.

Administration of the pharmaceutical compositions of the present invention to a patient subject, preferably a mammal, more preferably a human, may be carried out using known procedures, at dosages and for periods of time effective to ensure expression of the modified receptor or the modified receptor and nucleic acid in the subject in the subject. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily, or the dose may be proportionally reduced as indicated by the exigencies of the therapeutic situation.

Routes of administration of the disclosed compositions (containing the modified plant virus) include inhalational, oral, nasal, rectal, parenteral, sublingual, transdermal, transmucosal (e.g., sublingual, lingual, (trans)buccal, (trans)urethral, vaginal (e.g., trans- and perivaginally), (intra) nasal, and (trans) rectal), intravesical, intrapulmonary, intraduodenal, intragastrical, intrathecal, subcutaneous, intramuscular, intradermal, intra-arterial, intravenous, intrabronchial, inhalation, and topical administration. Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein. In one embodiment, the modified plant virus treatment comprises an administration route selected from the group consisting of inhalation, oral, rectal, vaginal, parenteral, topical, transdermal, pulmonary, intranasal, buccal, ophthalmic, intra-hepatic arterial, intrapleural, intrathecal, intra-tumoral, intravenal, and any combination thereof.

### E. Methods of Using the Modified Receptor and/or Modified Plant Virus

The disclosure also provides for methods of ushing the modified receptor and/or the modified plant virus. One aspect of the disclosure is directed to methods of transiently expressing a receptor for a modified plant virus in a mammalian cell. Another aspect of the disclosure is directed to methods of delivering a nucleotide sequence (such as a nucleotide sequence capable of exhibiting a therapeutic effect in a mammalian cell) to a mammalian cell. Yet another aspect of the disclosure is directed to a method of expressing a gene in a mammalian cell.

One embodiment of the disclosure is a method of transiently expressing a receptor for a modified plant virus in a mammalian cell, comprising delivering a composition comprising the modified receptor for the modified plant virus nucleotide to the mammalian cell. In the methods, the mammalian cell transiently expresses the modified receptor for the modified plant virus upon delivery of the modified receptor for the modified plant virus nucleotide.

In another embodiment, the method of transiently expressing a receptor for a modified plant virus in a mammalian cell of interest comprises contacting a mammalian cell of interest with a lipid nanoparticle of comprising the modified receptor for the modified plant virus nucleotide under conditions allowing delivery of the modified receptor for the modified plant virus nucleotide into the mammalian cell. In the methods, the mammalian cell transiently expresses receptor for the modified plant virus upon delivery of the modified receptor for the modified plant virus nucleotide, and the nanoparticle is configured to be selective for a specific mammalian cell. In certain embodiments, the nanoparticle is a lipid nanoparticle.

Another embodiment of the disclosure is directed to methods of delivering a nucleotide sequence capable of exhibiting a therapeutic effect in a mammalian cell to a mammalian cell, which transiently expresses the receptor for the modified plant virus comprising contacting the mammalian cell with a modified plant virus comprising a plant virus nucleotide sequence and a nucleotide sequence capable of exhibiting a therapeutic effect in a mammalian cell, wherein the virus is capable of transfecting a mammalian cell modified to express a receptor for the modified plant virus and wherein the mammalian cell expresses the nucleotide sequence capable of exhibiting a therapeutic effect upon transfection. In certain embodiments of the methods, any nucleotide sequence of interest may be used.

Yet another embodiment of the disclosure is directed to methods of expressing a gene in a mammalian cell. The methods include contacting a mammalian cell, that transiently expresses receptor for the modified plant virus with a modified plant virus comprising a plant virus nucleotide sequence and the gene, w wherein the virus is capable of transfecting the cell by virtue of the cell's expression of receptor for the modified plant virus, and wherein the cell expresses the gene upon transfection.

One embodiment of the disclosure directed to method of delivering a nucleotide sequence capable of exhibiting a therapeutic effect in a mammalian cell to a mammalian cell including:
contacting a mammalian cell with a composition comprising the modified receptor for the modified plant virus nucleotide under conditions allowing delivery of the nucleotide into the mammalian cell, so that the mammalian cell transiently expresses the modified receptor for the modified plant virus upon delivery of the modified receptor for the modified plant virus nucleotide; and
contacting the mammalian cell transiently expressing the receptor for the modified plant virus with a modified plant virus comprising a plant virus nucleotide sequence and a nucleotide sequence capable of exhibiting a therapeutic effect in a mammalian cell.
   In certain embodiments, the modified plant virus transfects the mammalian cell with the nucleotide sequence capable of exhibiting a therapeutic effect in a mammalian cell. In one embodiment, the nucleotide sequence capable of exhibiting a therapeutic effect is a mammalian gene. In another embodiment, the nucleotide sequence capable of exhibiting a therapeutic effect is a nuclease such as e.g. a CRISPR/cas construct, TALENS, or ZFNs.

In certain embodiments, the methods may be used to introduce the modified receptor or the modified receptor and the modified plant virus into the cell of a patient in need of a therapeutic nucleic acid. In certain embodiments, the patient may have antibodies against an adenoviral vector. In other embodiments, the patient is resistant or has become resistant to treatment with an adenoviral vector.

In yet another embodiment, the disclosure is directed to methods of expressing a gene in a mammalian cell comprising:
contacting a mammalian cell with a composition comprising the modified receptor for the modified plant virus nucleotide under conditions allowing delivery of the nucleotide into the mammalian cell so that the mammalian cell transiently expresses the modified receptor for the modified plant virus upon delivery of the modified receptor for the modified plant virus nucleotide; and
contacting the mammalian cell transiently expressing the receptor for the modified plant virus with a modified plant virus comprising a plant virus nucleotide sequence and a gene of interest, whereby the modified plant virus transfects the mammalian cell with the gene, and wherein the mammalian cell expresses the gene after transfection.

In yet another embodiment, the disclosure provides for method of selectively delivering a gene to a mammalian cell of interest comprising:
contacting a mammalian cell with a composition comprising a lipid nanoparticle and the modified receptor for the modified plant virus nucleotide under conditions allowing delivery of the modified receptor for the modified plant virus nucleotide into the mammalian cell, wherein the mammalian cell transiently expresses the modified receptor for the modified plant virus upon delivery of the modified receptor for the modified plant virus nucleotide; and
contacting the mammalian cell transiently expressing receptor for the modified plant virus with a modified plant virus comprising a plant virus nucleotide sequence and the gene. The methods of selectively delivering the gene require a lipid nanoparticle that is configured to be selective for the mammalian cell of interest. In one embodiment, the modified plant virus transfects the cell with the gene.

In yet another embodiment, the disclosure provides for methods of selectively expressing a nucleotide in a mammalian cell comprising:
contacting a mammalian cell with a composition comprising a lipid nanoparticle and the modified receptor for the modified plant virus nucleotide under conditions allowing delivery of the modified receptor for the modified plant virus nucleotide into the mammalian cell, wherein the mammalian cell transiently expresses the modified receptor for the modified plant virus upon delivery of the modified receptor for the modified plant virus nucleotide; and
contacting the mammalian cell transiently expressing receptor for the modified plant virus with a modified plant virus comprising a plant virus nucleotide sequence and the gene, wherein the modified plant virus transfects the mammalian cell with the gene, and wherein the mammalian cell expresses the nucleotide after transfection.
   The methods require a lipid nanoparticle configured to be selective for the mammalian cell of interest.

In certain embodiments of the disclosure, the plant virus nucleotide sequence comprises the nucleotide sequence of SEQ ID NO: 1-6 or a fragment thereof capable of transfecting a mammalian cell that has been modified to express receptor for the modified plant virus. In other embodiments, the modified plant virus comprises a plant virus nucleotide sequence into with a nucleotide sequence capable of exhibiting a therapeutic effect in a mammalian cell has been inserted.

In one embodiment, the methods use a plant virus nucleotide sequence having the nucleotide sequence of SEQ ID NO: 1-3 or 6 or a fragment thereof capable of transfecting a mammalian cell that has been modified to express receptor for the modified plant virus. In some embodiments, the modified plant virus comprises a first plant virus nucleotide sequence and a second plant virus nucleotide sequence into with a nucleotide sequence capable of exhibiting a therapeutic effect in a mammalian cell has been inserted.

For example, in one embodiment, the first plant virus nucleotide sequence comprises the nucleotide sequence of SEQ ID NO: 4 or a fragment thereof capable of transfecting a mammalian cell that has been modified to express receptor for the modified plant virus. In another embodiment, the second plant virus nucleotide sequence comprises the nucleotide sequence of SEQ ID NO: 5 or a fragment thereof.

The methods of the disclosure may be used with any kind of mammalian cell. In certain embodiments, the mammalian cell is a human cell. In one embodiment, the human cell is from a human having antibodies against and an adenoviral vector. In another embodiment, the mammalian cell is from a human patient that is resistant or has become resistant to treatment with an adenoviral vector.

### F. Kits

In other aspects the disclosure provides for kits of delivery of therapeutic genes comprising the modified plant virus and the modified receptor for the modified plant virus. In one embodiment, the kit comprises a pharmaceutical composition containing the modified plant virus and a pharmaceutical composition comprising the modified receptor. In certain embodiments, the kits include instructions for use.

Aspects. The present disclosure also pertains to and includes at least the following aspects:

Aspect 1. A modified plant virus comprising a plant virus nucleotide sequence and a nucleotide sequence capable of exhibiting a therapeutic effect in a mammalian cell, wherein the virus is capable of transfecting a mammalian cell, wherein the mammalian cell is modified to express a receptor for the modified plant virus.

Aspect 2. The modified plant virus of claim 1, wherein the nucleotide sequence capable of exhibiting a therapeutic effect is a mammalian gene.

Aspect 3. The modified plant virus of aspect 1, wherein the nucleotide sequence capable of exhibiting a therapeutic effect is a nuclease.

Aspect 4. The modified plant virus of aspect 3, wherein the nuclease is a CRISPR/cas construct, TALENS, or ZFNs.

Aspect 5. The modified plant virus of any one of aspects 1-4, wherein the plant virus nucleotide sequence comprises one or more of ORF I, ORF II, ORF III, ORF IV, ORF V, ORF VI, ORF VII and ORF VIII.

Aspect 6. The modified plant virus of any one of aspects 1-5, wherein the plant virus is Cauliflower Mosaic virus, Tomato Yellow Leaf Curl Virus (TYLCV) or Turnip Yellow Virus (TuYV).

Aspect 7. The modified plant virus of any one of aspects 1-5, wherein the nucleotide sequence capable of exhibiting a therapeutic effect in a mammalian cell is present within the plant virus nucleotide sequence.

Aspect 8. The modified plant virus of any one of aspects 1-5, wherein the plant virus comprises a first plant virus sequence and a second plant virus sequence and wherein the nucleotide sequence capable of exhibiting a therapeutic effect in a mammalian cell is present within the second plant virus nucleotide sequence.

Aspect 9. The modified plant virus of any one of aspects 1-6, wherein the plant virus nucleotide sequence comprises the nucleotide sequence of SEQ ID NO: 1-6 or a fragment thereof capable of transfecting a mammalian cell that has been modified to express a receptor for the modified plant virus.

Aspect 10. The modified plant virus of aspect 9, wherein the nucleotide sequence capable of exhibiting a therapeutic effect in a mammalian cell is present within the plant virus sequence of SEQ ID NO: 1-3, 5 or 6.

Aspect 11. The modified plant virus of aspect 9, wherein the nucleotide sequence capable of exhibiting a therapeutic effect in a mammalian cell is present within the plant virus sequence of SEQ ID NO: 2 at nucleotides 3424-6101.

Aspect 12. The modified plant virus of aspect 9, wherein the nucleotide sequence capable of exhibiting a therapeutic effect in a mammalian cell is present within the plant virus sequence of SEQ ID NO: 3 at nucleotides 1596-6101.

Aspect 13. The modified plant virus of aspect 9, wherein the nucleotide sequence capable of exhibiting a therapeutic effect in a mammalian cell is present within the plant virus sequence of SEQ ID NO: 5 at position 875.

Aspect 14. The modified plant virus of aspect 9, wherein the nucleotide sequence capable of exhibiting a therapeutic effect in a mammalian cell is present within the plant virus sequence of SEQ ID NO: 6 at position 4946.

Aspect 15. A pharmaceutical composition comprising the modified plant virus of any one of aspects 1 to 14 and a pharmaceutically acceptable carrier.

Aspect 16. A plant cell comprising the modified plant virus of any one of aspects 1 to 14.

Aspect 17. A modified receptor for a modified plant virus, comprising the amino acid sequence of SEQ ID NO: 7, 10, or 13 or a fragment thereof.

Aspect 18. The modified receptor for the modified plant virus of aspect 17, wherein the modified receptor or the fragment thereof renders a mammalian cell susceptible to infection with the modified plant virus.

Aspect 19. The modified receptor for the modified plant virus of aspect 17, wherein the modified receptor comprises an amino acid sequence at least 85%, 90%, 95%, 96%, 97%, 98% or 99% identical to SEQ ID NO: 7, 10, or 13.

Aspect 20. The modified receptor for the modified plant virus of aspects 17, wherein the fragment comprises an extracellular domain of the receptor.

Aspect 21. The modified receptor for the modified plant virus of aspect 17, wherein the fragment comprises amino acids 103 to 504 of SEQ ID NO: 4.

Aspect 22. The modified receptor for the modified plant virus of aspect 17, wherein the fragment comprises a peptide having at least 85%, 90%, 95%, 96%, 97%, 98% or 99% identity to amino acids 103 to 504 of SEQ ID NO: 4.

Aspect 23. The modified receptor for the modified plant virus of aspect 17, wherein the fragment comprises amino acids 35-268 of SEQ ID NO: 10.

Aspect 24. The modified receptor for the modified plant virus of aspect 17, wherein the fragment comprises a peptide having at least 85%, 90%, 95%, 96%, 97%, 98% or 99% identity to amino acids 35-268 of SEQ ID NO: 10.

Aspect 25. The modified receptor for the modified plant virus of aspect 17, wherein the fragment comprises amino acids 35-653 of SEQ ID NO: 13.

Aspect 26. The modified receptor for the modified plant virus of aspect 17, wherein the fragment comprises a peptide having at least 85%, 90%, 95%, 96%, 97%, 98% or 99% identity to amino acids 35-653 of SEQ ID NO: 13.

Aspect 27. The modified receptor for the modified plant virus of any one of aspects 20-26, wherein the fragment renders a mammalian cell susceptible to infection with the modified plant virus.

Aspect 28. The modified receptor for the modified plant virus of any one of aspects 19-27, wherein the fragment further comprises GPI anchor and/or a signal peptide.

Aspect 29. The modified receptor for the modified plant virus of aspect 17 or aspect 18, wherein the modified receptor consists of the amino acid sequence of SEQ ID NO: 7, 10, or 13, or a fragment thereof.

Aspect 30. A modified receptor for a modified plant virus, wherein the modified receptor is encoded by the nucleotide sequence of SEQ ID NO: 8, 9, 11, 12, 14, or 15, or a fragment thereof.

Aspect 31. The modified receptor for the modified plant virus, wherein the modified receptor or fragment thereof renders a mammalian cell susceptible to infection with the modified plant virus.

Aspect 32. The modified receptor for the modified plant virus according to aspect 30 or aspect 31, wherein the modified receptor is encoded by a nucleotide sequence having at least 85%, 90%, 95%, 96%, 97%, 98% or 99% identity to SEQ ID NO: 8, 9, 11, 12, 14, or 15.

Aspect 33. A modified receptor for a modified plant virus nucleotide, wherein the modified plant virus nucleotide comprises the nucleotide sequence of SEQ ID NO: 8, 9, 11, 12, 14, or 15, or a fragment thereof.

Aspect 34. The modified receptor for the modified plant virus nucleotide of aspect 33, wherein the modified receptor or fragment thereof renders a mammalian cell susceptible to infection with the modified plant virus.

Aspect 35. The modified receptor for the modified plant virus nucleotide of aspect 34, wherein the modified plant virus nucleotide consists of SEQ ID NO: 8, 9, 11, 12, 14, or 15, or a functional fragment thereof.

Aspect 36. A modified receptor for a modified plant virus nucleotide, wherein the modified plant virus nucleotide comprises the nucleotide having at least 85%, 90%, 95%, 96%, 97%, 98% or 99% identity to SEQ ID NO: 8, 9, 11, 12, 14, or 15, or a functional fragment thereof

Aspect 37. The modified receptor for the modified plant virus nucleotide of any one of aspects 34-36, wherein the nucleotide encoding the receptor is a codon optimized for human cells.

Aspect 38. A modified receptor for a modified plant virus nucleotide selected from the group consisting of:
(a) a modified receptor for the modified plant virus nucleotide, wherein the modified plant virus nucleotide comprises the nucleotide sequence of SEQ ID NO: 9, 12, or 15;
(b) a modified receptor for the modified plant virus nucleotide, wherein the modified plant virus nucleotide consists of the nucleotide sequence of SEQ ID NO: 9, 12, or 15;
(c) a modified receptor for the modified plant virus nucleotide, wherein the modified plant virus nucleotide comprises a functional fragment of the nucleotide sequence of SEQ ID NO: 9, 12, or 15, wherein the functional fragment is capable of rendering a mammalian cell susceptible to infection with plant virus or a derivative thereof; and
(d) combinations thereof.

Aspect 39. The modified receptor for the modified plant virus nucleotide of any one of aspects 34-38, wherein the modified plant virus comprises a modified plant virus of any one of aspects 1 to 14.

Aspect 40. The modified receptor for the modified plant virus nucleotide of aspects 38 or 39, wherein the nucleotide is a codon optimized for human cells.

Aspect 41. The cell or vector comprising the modified receptor for the modified plant virus nucleotide of any one of aspects 34-40.

Aspect 42. A pharmaceutical composition comprising the modified receptor for the modified plant virus nucleotide of any one of aspects 34-40 and a pharmaceutically acceptable carrier.

Aspect 43. The pharmaceutical composition of aspect 42, wherein the composition comprises the modified receptor for the modified plant virus nucleotide of any one of aspects 24 to 26.

Aspect 44. The pharmaceutical composition of aspect 43, wherein the composition comprises a lipid nanoparticle.

Aspect 45. The pharmaceutical composition of any one of aspects 42-44, wherein the composition is formulated for delivering the modified receptor for the modified plant virus nucleotide into a mammalian cell.

Aspect 46. The pharmaceutical composition of aspect 44, wherein the pharmaceutical composition comprises a lipid nanoparticle comprising the modified receptor for the modified plant virus nucleotide.

Aspect 47. A kit for delivering a therapeutic gene of interest comprising the pharmaceutical composition of aspect 15 and the pharmaceutical composition of any one of aspects 42-46.

Aspect 48. The kit of aspect 47 further comprising instructions for use.

Aspect 49. A method of transiently expressing a receptor for a modified plant virus in a mammalian cell, comprising delivering a composition comprising the modified receptor for the modified plant virus nucleotide of any one of aspects 33-40 into the mammalian cell, wherein the mammalian cell transiently expresses the modified receptor for the modified plant virus upon delivery of the modified receptor for the modified plant virus nucleotide.

Aspect 50. A method of transiently expressing a receptor for a modified plant virus in a mammalian cell of interest, comprising contacting a mammalian cell of interest with a lipid nanoparticle comprising the modified receptor for the modified plant virus nucleotide of any one of aspects 38-40 under conditions allowing delivery of the modified receptor for the modified plant virus nucleotide into the mammalian cell, wherein the mammalian cell transiently expresses receptor for the modified plant virus upon delivery of the modified receptor for the modified plant virus nucleotide, and wherein the nanoparticle is configured to be selective for the mammalian cell.

Aspect 51. The method of aspect 50, wherein the nanoparticle is a lipid nanoparticle.

Aspect 52. A method of delivering a nucleotide to a mammalian cell comprising contacting the mammalian cell with a modified plant virus comprising a plant virus nucleotide sequence and a nucleotide sequence capable of exhibiting a therapeutic effect in a mammalian cell, wherein the virus is capable of transfecting a mammalian cell that has been modified to express a receptor for the modified plant virus and wherein the mammalian cell expresses the nucleotide sequence capable of exhibiting a therapeutic effect upon transfection, and wherein the cell transiently expresses the receptor for the modified plant virus.

Aspect 53. A method of expressing a gene in a mammalian cell comprising contacting a mammalian cell with a modified plant virus comprising a plant virus nucleotide sequence and the gene, wherein the cell transiently expresses receptor for the modified plant virus, wherein the virus is capable of transfecting the cell by virtue of the cell's expression of receptor for the modified plant virus, and wherein the cell expresses the gene upon transfection.

Aspect 54. A method of delivering a nucleotide sequence capable of exhibiting a therapeutic effect in a mammalian cell to a mammalian cell comprising:
contacting a mammalian cell with a composition comprising the modified receptor for the modified plant virus nucleotide of any one of aspects 17-40 under conditions allowing delivery of the modified receptor for the modified plant virus nucleotide into the mammalian cell, wherein the mammalian cell transiently expresses the modified receptor for the modified plant virus upon delivery of the modified receptor for the modified plant virus nucleotide; and
contacting the mammalian cell transiently expressing receptor for the modified plant virus with a modified plant virus comprising a plant virus nucleotide sequence and a nucleotide sequence capable of exhibiting a therapeutic effect in a mammalian cell.

Aspect 55. The method of aspect 54, wherein the modified plant virus transfects the mammalian cell with the nucleotide sequence capable of exhibiting a therapeutic effect in a mammalian cell.

Aspect 56. The method of aspect 54, wherein the nucleotide sequence capable of exhibiting a therapeutic effect is a mammalian gene.

Aspect 57. The method of aspect 56, wherein the nucleotide sequence capable of exhibiting a therapeutic effect is a nuclease.

Aspect 58. The method of aspect 57, wherein the nuclease is a CRISPR/cas construct, TALENS, or ZFNs.

Aspect 59. A method of expressing a gene in a mammalian cell comprising:
contacting a mammalian cell with a composition comprising the modified receptor for the modified plant virus nucleotide of any one of aspects 17-40 under conditions allowing delivery of the modified receptor for the modified plant virus nucleotide into the mammalian cell, wherein the mammalian cell transiently expresses the modified receptor for the modified plant virus upon delivery of the modified receptor for the modified plant virus nucleotide; and
contacting the mammalian cell transiently expressing receptor for the modified plant virus with a modified plant virus comprising a plant virus nucleotide sequence and a gene of interest, wherein the modified plant virus transfects the mammalian cell with the gene, and wherein the mammalian cell expresses the gene after transfection.

Aspect 60. A method of selectively delivering a gene to a mammalian cell of interest comprising:
contacting a mammalian cell with a composition comprising a lipid nanoparticle and the modified receptor for the modified plant virus nucleotide of any one of aspects 17-40 under conditions allowing delivery of the modified receptor for the modified plant virus nucleotide into the mammalian cell, wherein the mammalian cell transiently expresses the modified receptor for the modified plant virus upon delivery of the modified receptor for the modified plant virus nucleotide; and
contacting the mammalian cell transiently expressing receptor for the modified plant virus with a modified plant virus comprising a plant virus nucleotide sequence and the gene, wherein the lipid nanoparticle is configured to be selective for the mammalian cell of interest.

Aspect 61. The method of aspect 60, wherein the modified plant virus transfects the cell with the gene.

Aspect 62. A method of selectively expressing a nucleotide in a mammalian cell comprising:
contacting a mammalian cell with a composition comprising a lipid nanoparticle the modified receptor for the modified plant virus nucleotide of any one of aspects 17-40 under conditions allowing delivery of the modified receptor for the modified plant virus nucleotide into the mammalian cell, wherein the mammalian cell transiently expresses the modified receptor for the modified plant virus upon delivery of the modified receptor for the modified plant virus nucleotide; and
contacting the mammalian cell transiently expressing receptor for the modified plant virus with a modified plant virus comprising a plant virus nucleotide sequence and the gene, wherein the modified plant virus transfects the mammalian cell with the gene, and wherein the mammalian cell expresses the nucleotide after transfection, wherein the lipid nanoparticle is configured to be selective for the mammalian cell of interest.

Aspect 63. The method of any one of aspects 52-62, wherein the plant virus nucleotide sequence comprises the nucleotide sequence of SEQ ID NO: 1-6 or a fragment thereof capable of transfecting a mammalian cell that has been modified to express a receptor for the modified plant virus.

Aspect 64. The method of any one of aspects 52-62, wherein the modified plant virus comprises a plant virus nucleotide sequence into with a nucleotide sequence capable of exhibiting a therapeutic effect in a mammalian cell has been inserted.

Aspect 65. The method of aspect 64, wherein the plant virus nucleotide sequence comprises the nucleotide sequence of SEQ ID NO: 1-3 or 6 or a fragment thereof capable of transfecting a mammalian cell that has been modified to express a receptor for the modified plant virus.

Aspect 66. The method of any one of aspects 52-62, wherein the modified plant virus comprises a first plant virus nucleotide sequence and a second plant virus nucleotide sequence into with a nucleotide sequence capable of exhibiting a therapeutic effect in a mammalian cell has been inserted.

Aspect 67. The method of aspect 66, wherein the first plant virus nucleotide sequence comprises the nucleotide sequence of SEQ ID NO: 4 or a fragment thereof capable of transfecting a mammalian cell that has been modified to express a receptor for the modified plant virus.

Aspect 68. The method of aspect 66, wherein the second plant virus nucleotide sequence comprises the nucleotide sequence of SEQ ID NO: 5 or a fragment thereof.

Aspect 69. The method of any one of aspects 52-68, wherein the mammalian cell is a human cell.

Aspect 70. The method of aspect 69, wherein the human cell is from a human having antibodies against and an adenoviral vector.

Aspect 71. The method of aspect 69, wherein the mammalian cell is from a human patient that is resistant or has become resistant to treatment with an adenoviral vector.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1

### General Principle of Using Plant Viruses as a Vector for Delivery of Nucleic Acids Into Mammalian Cells

Plant viruses do not enter mammalian cells because the mammalian cells do not have the necessary receptors for the virus. Plant viruses due to their size are able to carry a large nucleic acid payload that may render them useful for delivery of a nuclei acid, as a therapeutic gene into a mammalian cell.

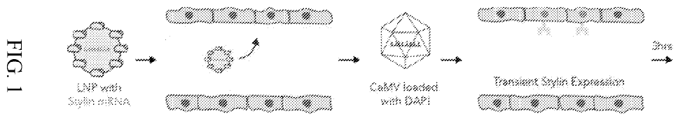

A schematic of a general method of using plant viruses to deliver a nucleic acid to a mammalian cell is shown in FIG. 1. In step one, a lipid nanoparticle (LNP) delivers mRNA encoding for the viral receptor. This viral receptor only lasts a few hours. Within those hours, the modified plant virus is delivered, which only enters the cells targeted with the LNP containing the mRNA encoding the receptor of the viral vector. With reference to the schematic in FIG. 1, the viral receptor is Stylin, and the modified plant virus is Cauliflower Mosaic virus which has been loaded with DAPI.

### Example 2

### In Vitro and In Vivo Delivery of Plant Viruses to Mammalian Cells

To confirm that the general two-step method shown in FIG. 1 is indeed feasible, initial studies were carried out using a virus that only enters strawberry cells (Strawberry Mild Yellow Edge Virus). In step 1, a clinically relevant LNP was used to deliver mRNA encoding a receptor for the virus that infects strawberry cells (Strawberry Mild Yellow Edge Virus); critically, this virus does not enter human cells on its own. The LNP-delivered mRNA is translated into the strawberry virus receptor on human cells for short (˜6 hours) and controllable periods of time. Second, the strawberry virus, which only enters cells expressing the strawberry virus receptor was administered. Notably, the receptor would only be expressed on the cells purposefully targeted with our LNP. Thus, by controlling which cell types express the receptor, the virus can be specifically targeted to a cell of interest.

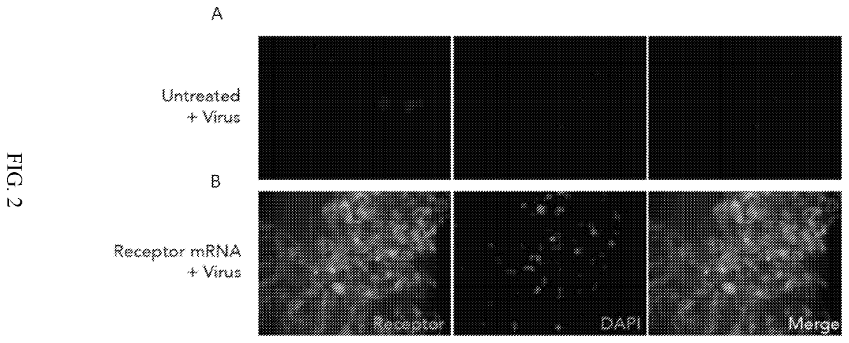

Significant data was generated suggesting it is indeed feasible to use a plant virus to deliver nucleic acids to a mammalian cell. This data was generated by encapsulating mRNA encoding the receptor for the virus in a lipid nanoparticle. The virus was then administered and observed to only enter cells the express the receptor. First, it was demonstrated that mRNA encoding viral proteins in cells can be delivered. Viral receptor delivery (FIG. 2A,B) and subsequent viral delivery into cells in vitro has been observed (see FIG. 2A, B). In one such experiment, a receptor for a virus using mRNA in mouse cells was transiently expressed. The plant virus entered cells with the receptor (FIG. 2B) but did not enter cells without the receptor (FIG. 2A). In FIG. 2A, the virus stained with DAPI (shown in blue) does not enter cells that do not express the receptor (shown in green). In FIG. 2B, the virus stained with DAPI enters cells that express the receptor. This is an important first step for making this approach work in vivo.

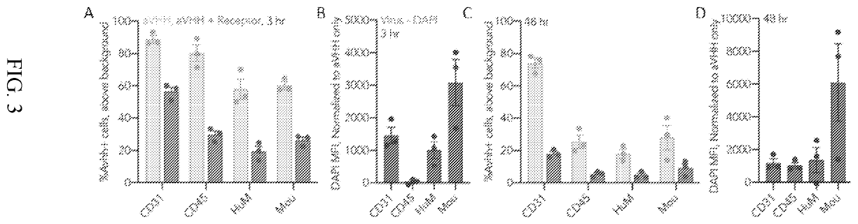

Second, a nanoparticle which delivers mRNA to cells in the liver (FIG. 3A-D) of humanized mice was identified (FIG. 3A-D). This demonstrates the potential this approach has to generate substantial value by enabling gene therapies in new cell types. Specifically, by pre-treating patients with LNP-Liver carrying the virus receptor, then delivering the virus, it is possible to carry out gene therapies targeting the liver. Similarly, by pre-treating patients with LNPs targeting other organs such as the lung or spleen carrying the virus receptor, then delivering the virus, gene therapies targeting the lung or spleen are possible. Thus, gene therapies can be targeted specifically to multiple cell types. Third, delivery of mRNA encoding the receptor (FIG. 3A, C) and virus (FIG. 3B, D) into ‘humanized’ mice, which are mice with human or monkey cells in them, has been demonstrated.

Specifically, FIG. 3A-3D show delivery of mRNA encoding viral receptor and successful delivery using LNP-liver in humanized mice. mRNA encoding viral receptor can be delivered to humanized mouse cells and the receptor expression is evident after 3 hours and 48 hours (FIG. 3A, 3C). Virus stained with DAPI enters cells that receive aVHH+viral receptor but does not enter cells that receive aVHH only (control); virus can be seen after 3 hours (FIG. 3B) and 48 hours (FIG. 3D). Therefore it has been demonstrated the ability to deliver the receptor and virus into human or monkey cells within a living animal.

### Example 3

### Investigation of CAMV as a Vector for Mammalian Cells

Based on the testing discussed in Example 2 above, cauliflower mosaic virus (CaMV) was investigated for its suitability as a vector for mammalian cells. CAMV is a plant virus that is unable to infect mammalian cells. CAMV uses reverse transcription for genome amplification (see e.g. Mikhail Schepetilnikov, Lyubov Ryabova, in Plant Virus-Host Interaction, 2014). Due to its size and the presence of a reverse transcriptase, the use of CAMV as a vector to selectively deliver nucleic acids to mammalian cells was investigated.

The same general protocol as discussed in Example 1 was used. As shown in FIG. 1, CaMV requires the receptor stylin for infection.

For this study, the stylin receptor was optimized using a CD55 CPI anchor. The amino acid sequence of the modified stylin receptor (Stylin-1 with CD55 CPI anchor) (SEQ ID NO: 7) is shown below. Furthermore, this study used wild-type CaMV (SEQ ID NO: 1).

Stylin-1 with CD55 CPI anchor
(SEQ ID NO: 7)
IRGRAPRTRPSPPPPLLPLLSLSLLLLSPTVRGQVTFAVSSLLLAVVAV
SAYPASLNPESRAAILVQDSAPNADGSFKNNFQTENGIKQESVGYLKAG
PEGPVAVFQGASAYVAPDGQTIQIGYIADENGYQPYGAHLPTPPPIPAE
IQESLRYLASLPSTPEPKYQGGDRYIYGHTCLITLTVLHVMLSLIGYLT
This modified stylin receptor has the DNA
sequence of SEQ ID NO: 8 and the RNA sequence of
SEQ ID NO: 9, both of which are also shown below.
Stylin-1 with CD55 CPI anchor (DNA)
(SEQ ID NO: 8)
ATAAGAGGCCGCGCCCCACGCACCCGCCCTAGTCCCCCTCCTCCTTTGT
TGCCACTTCTTTCTCTGTCCCTTCTCCTCCTCTCTCCTACTGTGAGGGG
TCAGGTCACTTTTGCCGTATCGTCGTTGCTGTTAGCTGTCGTCGCCGTC
AGCGCCTACCCCGCATCACTGAACCCGGAATCCAGAGCCGCCATCTTGG
TCCAAGATTCAGCACCCAACGCCGATGGATCATTCAAGAACAATTTCCA
AACCGAAAACGGAATCAAACAAGAATCAGTCGGATACTTGAAGGCTGGC
CCAGAAGGACCCGTAGCTGTGTTCCAGGGAGCTTCTGCGTACGTCGCCC
CAGACGGCCAGACCATCCAAATCGGATACATCGCCGACGAGAACGGTTA
CCAGCCGTACGGCGCTCATTTGCCCACTCCACCACCAATCCCAGCTGAG
ATCCAAGAGTCGCTCAGATACCTCGCCTCTCTGCCCAGCACCCCCGAAC
CAAAATACCAGGGAGGAGACCGGTACATATACGGGCATACCTGCTTGAT
AACTCTGACAGTGCTGCATGTGATGCTTTCACTTATTGGTTACTTGACT
TGA
Stylin-1 with CD55 CPI anchor (RNA)
(SEQ ID NO: 9)
AUAAGAGGCCGCGCCCCACGCACCCGCCCUAGUCCCCCUCCUCCUUUGU
UGCCACUUCUUUCUCUGUCCCUUCUCCUCCUCUCUCCUACUGUGAGGGG
UCAGGUCACUUUUGCCGUAUCGUCGUUGCUGUUAGCUGUCGUCGCCGUC
AGCGCCUACCCCGCAUCACUGAACCCGGAAUCCAGAGCCGCCAUCUUGG
UCCAAGAUUCAGCACCCAACGCCGAUGGAUCAUUCAAGAACAAUUUCCA
AACCGAAAACGGAAUCAAACAAGAAUCAGUCGGAUACUUGAAGGCUGGC
CCAGAAGGACCCGUAGCUGUGUUCCAGGGAGCUUCUGCGUACGUCGCCC
CAGACGGCCAGACCAUCCAAAUCGGAUACAUCGCCGACGAGAACGGUUA
CCAGCCGUACGGCGCUCAUUUGCCCACUCCACCACCAAUCCCAGCUGAG
AUCCAAGAGUCGCUCAGAUACCUCGCCUCUCUGCCCAGCACCCCCGAAC
CAAAAUACCAGGGAGGAGACCGGUACAUAUACGGGCAUACCUGCUUGAU
AACUCUGACAGUGCUGCAUGUGAUGCUUUCACUUAUUGGUUACUUGACU
UGA

A liver-cell specific LNP containing Stylin-1 was administered into humanized mice. 12-24 hours after administration of the LNP, the mice were administered CaMV. Three days after administration of CaMV, mice were sacrificed, perfused, and liver sections were taken out of the mice.

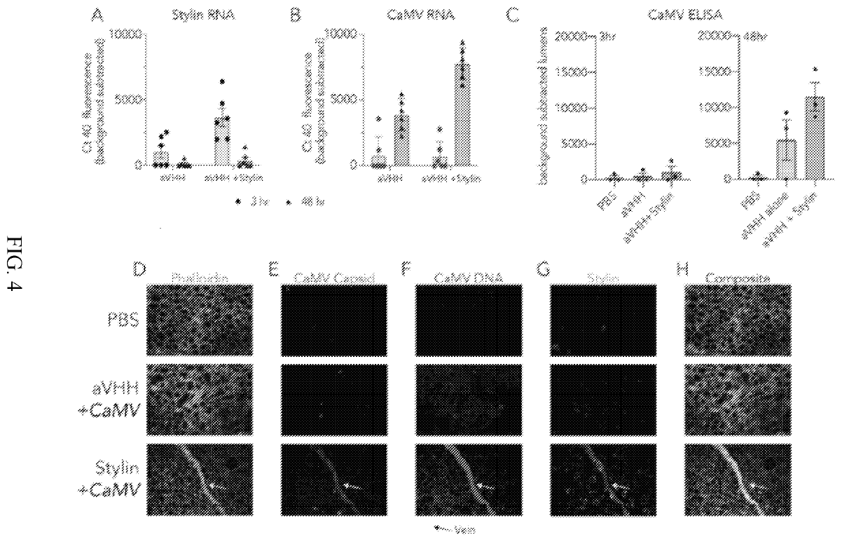

The liver sections of humanized mice were imaged and co-localization of Stylin receptor protein and CaMV capsid protein as well as payload (e.g. DAPI) was show. This suggests the ability to deliver Stylin mRNA and subsequently infect transfected cells with CaMV (FIG. 4A-4H).

Specifically, FIG. 4A-4H show the quantification of CaMV levels using protein methods. FIG. 4A and FIG. 4B show quantitative-PCR (qPCR) measurements for Stylin and CaMV RNA at 3 and 48 hours after CaMV administration. FIG. 4C shows ELISA data showing CaMV protein levels 3 and 48 hours after administration. FIG. 4D-FIG. 4F show immunofluorescence imaging that stains for cell outlines using phalloidin (FIG. 4D), the CaMV capsid protein (FIG. 4E), CaMV containing DAPI (FIG. 4F), the Stylin protein. (FIG. 4G) and a composite image showing co-localization of Stylin protein and CaMV (FIG. 4H).

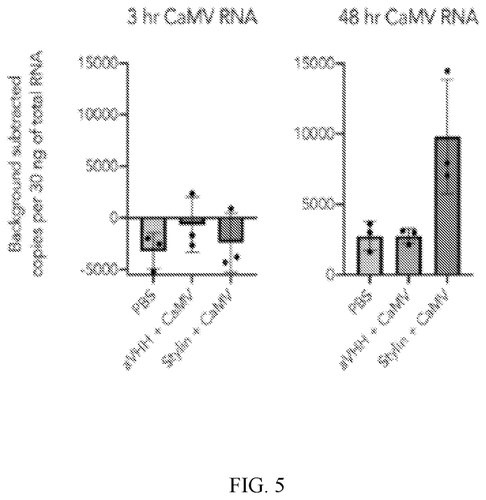

A ddPCR protocol was also developed to quickly determine the CaMV infection levels in liver tissue (FIG. 5). This protocol was observed to give much cleaner data than the qPCR data. In fact, background levels of CaMV, similar to those in PBS mice, were observed when mice were only pre-treated aVHH mRNA and much higher levels of CaMV infection when mice were pre-treated with Stylin mRNA as well.

Additionally, a scRNA sequencing experiment in humanized mice to determine cell type delivery of CaMV was performed. Stylin hybridoma cells were sequenced to create a recombinant antibody for Stylin detection with FACS and scRNA sequencing. This data is currently being analyzed to understand which cells were infected with CaMV and verify that they co-localize with cells that were pre-treated with Stylin mRNA.

This example established that when mammalian cells transiently express stylin, it is possible for the cells in vivo to be infected with CAMV. Accordingly, CAMV is a suitable vector for delivery of nucleic acid of interest to mammalian cells.

### Example 4

### Use of CAMV as a Vector to Deliver Nucleic Acids Into Mammalian Cells

Having established that wild-type CAMV can be used to in combination with a stylin derivate to infect mammalian cells in vivo, a similar study was carried out using a modified CAMV. For this study, CAMV was modified to encode GFP (see SEQ ID NO: 2 and SEQ ID NO: 3).

Specifically, a plant cell line (BY-2) was used to aid in production of CaMV. This cell line was used to produce CaMV encoding for a GFP functional protein (CaMV-GFP). In order to make CaMV-GFP, the BY-2 cell line needs to be digested into protoplasts and then transfected with a plasmid containing the CaMV-GFP construct as well as an accessory construct.

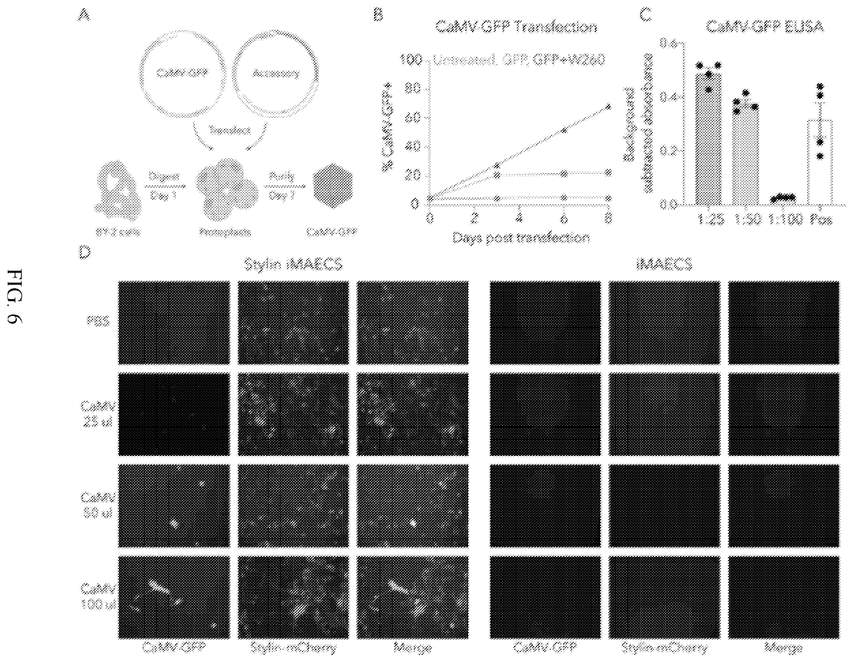

The percentage of GFP+ cells in BY-2 protoplasts was assessed and it was observed that the percentage of GFP+ cells increases every day post-transfection, suggesting that the BY-2 cells are producing new virus that is capable of infecting more and more BY-2 cells over time. As a control, cells were transfected with GFP only; these cells are not capable of producing new virus and their GFP expression stabilizes over time (See FIGS. 6A and 6B).

FIG. 6A-6D establish that it is possible to generate a plant cell (BY-2 cells) capable of producing CaMV encoding for GFP. FIG. 6A shows the general protocol for modifying plant cells to produce a modified CaMV such as CaMV encoding for GFP. BY-2 cells are digested into protoplasts. Protoplasts will only produce CaMV-GFP if they contain both the CaMV-GFP construct and the accessory construct. CaMV-GFP can be purified from these protoplasts and used for downstream experiments. FIG. 6B establishes that transfection of BY-2 cells with CaMV was successful. BY-2 cells continue to produce CaMV-GFP, as noted by the increase in the number of BY-2 cells that are GFP positive. Verification of the purification of CaMV-GFP from BY-2 cells was confirmed using an ELISA kit optimized for CaMV (FIG. 6C). Purified CaMV-GFP leads to GFP+ cells in iMAECs containing the Stylin receptor but not iMAECs without it (FIG. 6D).

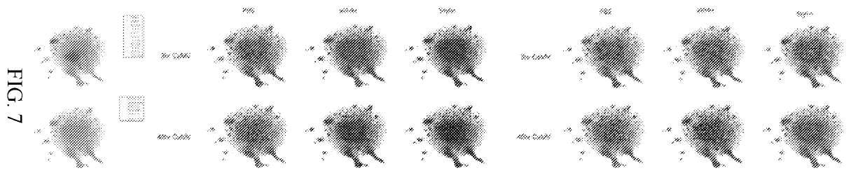

e CaMV-GFP was purified from the BY-2 cells and the presence of CaMV protein was confirmed using an ELISA (FIG. 7). Additionally, to validate that the purified CaMV could infect receptor (Stylin+) mammalian cells different amounts of purified CaMV (25 μL, 50 μL, 100 μL) were taken and put it onto immortalized mouse aortic endothelial (iMAEC) cells expressing Stylin on the surface and four days following transfection the cells were observed to be GFP+ (FIG. 7). As a control, iMAECs not expressing Stylin were transfected with CaMV-GFP and no GFP+ cells were observed, implying that the virus is entering cells through the Stylin receptor.

Single cell RNA sequencing data for humanized mice reveals CaMV RNA delivery in cell types containing Stylin protein. As a first step, this is indicatory of the ability to modulate the delivery of CaMV to cells that express Stylin and avoid off-target to cells that do not. CaMV RNA would only show up if CaMV were successfully delivered to the nucleus of a cell and translated into RNA. Single cell RNA sequencing bypasses our issues with staining for Stylin protein because it allows us to read out Stylin at the RNA level. As expected, abundant Stylin mRNA was observed to be present at 24 hours post-transfection with LNP in the 3-hour CaMV timepoint, and much less Stylin mRNA present 72 hours post-transfection in the 48-hour CaMV timepoint.

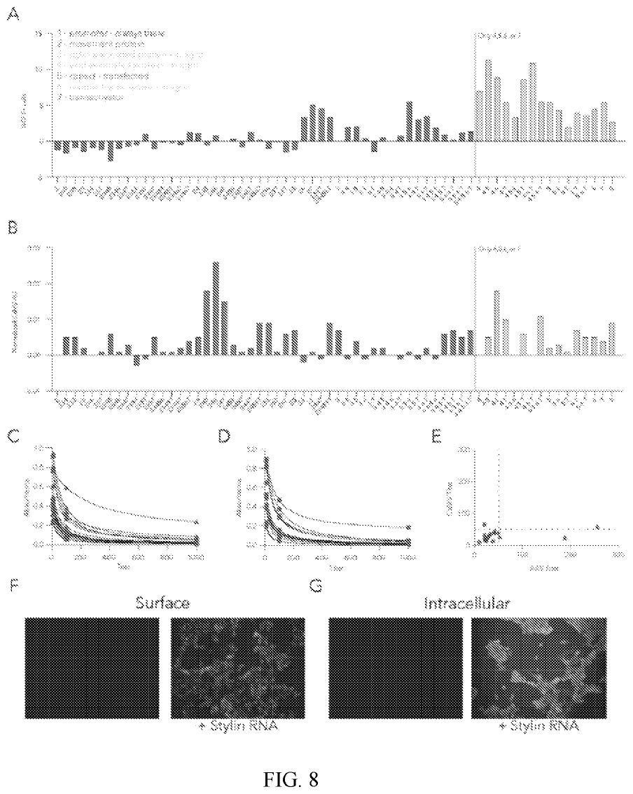

An additive experiment using each individual gene from CaMV was conducted to determine the minimal genes required for potent viral production. Genes 4, 5, 6, and 7 were observed to be required for making virus that led to high levels of reporter positive cells, and high levels of detectable CaMV (FIGS. 8A.B). While genes 2 and 3 also made high levels of detectable CaMV, they did not lead to high levels of reporter positive cells. It is believed that this is indicative of recombination. The presence of natural immunity to CaMV when compared to the commonly used infection vector AAV was also assessed. AAV was found to lead to higher titers as compared to CaMV in the same group of patient serum samples (FIGS. 8C-E). A new batch of Stylin mRNA was made, tested it in vitro, and observed to lead to the production of Stylin protein (FIGS. 8F,G).

FIG. 8A-8G show the results of checkerboard experiments to identify genes that are most important to produce functional CAMV. FIGS. 8A and B establish that components 4, 5 and 6 were most important for making functional virus that led to the most GFP+ cells. Titer levels for human serum patients against AAV and CaMV are shown in FIG. 8C and FIG. 8D, respectively. Only one patient had CaMV titer levels above the cutoff whereas two patients had AAV titer levels firmly above the cutoff (FIG. 8E). Stylin positive cells were stained on both the surface (FIG. 8F), and intracellularly (FIG. 8G) to confirm that the new batch of Stylin mRNA worked.

The testing shown in this example confirm that CaMV can be used in conjunction with a modified Stylin-1 receptor to selectively deliver a nucleic acid of interest to a mammalian cell transiently expressing the Stylin-1 receptor.

### Example 5

### Construction of Other Plant Virus and Plant Virus Receptor Constructs for Use as Vectors to Deliver Nucleic Acids to Mammalian Cells

Based on the testing shown in Examples 1-4, additional plant virus and plant virus receptor constructs were designed to vectors to selectively deliver nucleic acids of interest.

### Tomato Yellow Leaf Curl Virus and BtPGRP Constructs

Tomato yellow leaf curl virus (TYLCV) is a geminivirus (family Geminiviridae). While the virus can infect a relatively wide range of plant species, tomato is the host to which the virus is best adapted and that facilitates the build-up of the virus to high incidences in the field. Like CaMV, TYLCV does not infect mammalian cells.

“Peptidoglycan recognition proteins (PGRPs) are multifunctional pattern recognition proteins. Here, we report that a PGRP gene, BtPGRP, encodes a PGRP from the whitefly Bemisia tabaci (MEAM1) that binds and kills bacteria in vitro.” Wang et al., Sci Rep. 2016; 6: 37806 at Abstract. “BtPGRP has a TYLCV binding site to form a BtPGRP and TYLCV complex.” Wang et al. at 6. Accordingly, BtGRP was chosen as the receptor for TYLCV.

A construct of the BtGRP receptor containing the BtGRP receptor extracellular domain a signal peptide and a GPI anchor was generated. Specifically, a BtGRP receptor construct containing an extracellular domain of BtGRP, a CD55 signal peptide, and a CD55 GPI anchor was constructed. The nucleic acid and peptide sequences of this BtGRP receptor construct are shown below.

BtPGRP receptor with CD55 signal peptide and CD55
GPI anchor (amino acid sequence)
(SEQ ID NO: 10)
MIRGRAPRTRPSPPPPLLPLLSLSLLLLSPTVRGSQVFVDFFAKTHLMT
RLLVSVACPAILARDSWYASPATGPIDKYDPEQPPSMVIIHHSRLPPCS
TTESCIVRMLELQRLHQRDRHWFDIGFNFAIGGDGSVYEGRGWNQKPAA
VKNYNNKSINIAFLGDFSSSVPSAEMLKTARDLIDCGVRTGKISRDYKL
VGYSEQDVSSSVSGPSSSTSSPTSDSSDSSSSPSSFPSSLFSHLQSWPH
WSPMNLTDQAEPPTELKLALLVEGGDRYIYGHTCLITLTVLHVMLSLIG
YLT
BtPGRP receptor with CD55 signal peptide and CD55
GPI anchor (DNA sequence)
(SEQ ID NO: 11)
ATGATAAGAGGCCGCGCCCCACGCACCCGCCCTAGTCCCCCTCCTCCTT
TGTTGCCACTTCTTTCTCTGTCCCTTCTCCTCCTCTCTCCTACTGTGAG
GGGTAGCCAGGTTTTCGTGGATTTCTTTGCGAAGACGCACCTCATGACC
CGGCTGCTAGTCTCGGTGGCATGTCCGGCGATCCTAGCCCGAGATTCGT
GGTACGCGAGCCCTGCGACGGGCCCGATCGACAAATACGACCCCGAGCA
ACCTCCATCCATGGTCATCATCCACCACTCCCGACTTCCACCTTGCAGC
ACCACCGAAAGTTGTATCGTACGAATGCTAGAATTGCAGAGGCTACACC
AGAGAGATCGCCACTGGTTCGACATCGGTTTCAATTTTGCGATAGGTGG
CGATGGATCTGTTTATGAGGGACGAGGATGGAATCAAAAACCAGCGGCT
GTTAAAAATTACAATAACAAAAGCATCAACATAGCATTTCTCGGCGATT
TTAGCTCGAGCGTTCCCTCGGCGGAGATGCTGAAAACGGCGCGGGACCT
AATCGACTGCGGCGTGCGAACCGGGAAAATCTCGCGGGACTACAAGCTC
GTGGGCTACTCGGAGCAGGACGTGAGCTCGAGCGTGAGCGGGCCGAGCA
GCTCGACGTCATCGCCGACGTCAGACTCGTCCGACTCCTCGTCATCGCC
GTCGTCATTCCCGTCCTCCCTGTTCAGTCACCTCCAGAGCTGGCCGCAC
TGGTCCCCGATGAACCTCACCGACCAGGCCGAGCCGCCCACCGAGCTCA
AACTCGCCCTCCTCGTCGAAGGAGGAGACCGGTACATATACGGGCATAC
CTGCTTGATAACTCTGACAGTGCTGCATGTGATGCTTTCACTTATTGGT
TACTTGACTTGA

This BtGRP receptor can be used in conjunction with TYLCV to deliver a nucleic acid of interest to a cell transiently expressing the BtGRP receptor. The BtGRP receptor can be used in conjunction with a TYCLV full length genome fragment 1 of SEQ ID NO: 4 and a TYCLV transgene fragment of SEQ ID NO: 5.

Turnip Yellow Virus (TuYV) and mpEPH Constructs

“Turnip yellows virus (TuYV; family Solemoviridae, genus Polerovirus) is the most widespread and economically damaging virus of canola (Brassica napus L.) production in Australia.” Congdon et al., Plant Disease, Vo. 105, No. 9 (2021). Like CaMV and TYLCV, TuYV does not infect mammalian cells.

“The membrane-bound Ephrin receptor (Eph) is a aphid protein involved in the transmission of the Turnip yellows virus (TuYV, Polerovirus genus, Luteoviridae family) by Myzus persicae. The minor capsid protein of TuYV, essential for aphid transmission, was able to bind the external domain of Eph in yeast.” Mulot et al. Front Microb. 2018 Mar. 13; 9:457. Accordingly the Eph receptor was chosen as the receptor for TuYV.

A construct of the mpEPH receptor containing the mpEPH receptor extracellular domain, a signal peptide, and a GPI anchor was generated. Specifically, a mpEPH receptor construct containing an extracellular domain of mpEPH, a CD55 signal peptide and a CD55 GPI anchor was constructed. The nucleic acid and peptide sequences of this mpEPH receptor construct are shown below.

mpEPH receptor with CD55 signal peptide (1-102)and CD55 GPI anchor (1960-2046)
(amino acid sequence)
(SEQ ID NO: 13)
MIRGRAPRTRPSPPPPLLPLLSLSLLLLSPTVRGHKYHQHHCDDSSHHHHRHNHHHHLNRHH
RCCLAAVFLLLAVAIVSPGRAEHVVLLDTTTEPSLRWTTYPYGPDANAAGWVEESYINFEKG
INWRSYVVCDVTKQNVNNWVWTPFIERGLANLIYIEVKFTIRDCSLFPGYALSCKETFSLLYY
EFDVATREPPPWEPDSYKSVGRIAAGEGRFNANNEVVINTETKAVKVTKKGVYFAFRDQGA
CISIMAVKVYYIVCPEVVINFANFSATPTARELTQIEHATGKCVDNAEVVGGGAPTYLCKGD
GKWYLPSGGCKCKAGFEADIEAQTCIICPPGKYKYGVGDDKCQPCPAHSKAPDQGMSECRC
NTGYYRSPKDPKSVPCTQPPSAPQNLTVNFVDQSTVTLSWNPPNFLGGRTDIVYRVTCDMCG
PSVVFMPNNEVENDTKITISGLGPVTTYKFHVWAENGVSNLTSSENRQFVDIAVTTTEASVKS
ASVNNVRVMLVKASEITLSWDPPMASFFDSGDDDAAVEVYEVKFYPRGDESNVSNKLTADR
HMVFTALRPKTDYGFQVRAKTAHGWGEYSPTIYKTTGQLLGSGKNTQQQVQRKNGIKDDT
AYIGDEDNMEVRIIAGATVAVVVVLVVVIIMTVLFLGGDRYIYGHTCLITLTVLHVMLSLIGYLT
mpEPH receptor with CD55 signal peptide (1-102) and CD55 GPI anchor (1960-2046)
(DNA sequence)
(SEQ ID NO: 14)
ATGATAAGAGGCCGCGCCCCACGCACCCGCCCTAGTCCCCCTCCTCCTTTGTTGCCACTT
CTTTCTCTGTCCCTTCTCCTCCTCTCTCCTACTGTGAGGGGTCACAAGTATCACCAACACC
ACTGCGACGACAGCAGCCATCACCACCATCGGCACAACCACCATCATCACCTCAACCGT
CACCACCGATGCTGTCTCGCCGCGGTGTTTCTGCTGCTGGCCGTCGCGATCGTCAGCCCC
GGACGAGCCGAACACGTTGTACTTTTGGACACCACGACAGAGCCTAGTCTACGGTGGAC
TACGTACCCTTACGGACCGGACGCCAACGCAGCAGGGTGGGTCGAAGAGAGCTACATAA
ATTTTGAAAAGGGCATCAACTGGCGGTCGTACGTCGTGTGCGATGTCACCAAGCAAAAC
GTCAACAATTGGGTGTGGACGCCTTTCATAGAGCGCGGGCTGGCCAATCTCATTTACATC
GAAGTCAAGTTTACCATCCGAGATTGTTCGCTGTTCCCGGGGTACGCGCTGTCATGCAAA
GAAACGTTTTCACTACTCTACTATGAGTTTGACGTGGCCACCAGGGAACCACCGCCTTGG
GAACCTGACAGCTATAAATCTGTTGGTCGGATAGCTGCTGGTGAAGGGAGGTTCAACGC
TAATAACGAAGTGGTGATCAATACTGAGACAAAAGCTGTGAAAGTTACCAAAAAAGGAG
TGTACTTTGCCTTCAGGGACCAGGGCGCATGTATTTCCATCATGGCAGTCAAGGTGTATT
ACATCGTGTGCCCAGAAGTTGTGATAAACTTTGCAAACTTCTCTGCTACTCCAACTGCTA
GAGAACTTACCCAAATCGAACATGCCACCGGCAAATGTGTTGATAATGCTGAAGTGGTA
GGAGGTGGTGCTCCAACTTACCTGTGCAAAGGAGATGGAAAATGGTATCTACCGTCAGG
TGGATGCAAGTGCAAAGCTGGTTTTGAAGCAGACATAGAAGCGCAAACTTGTATAATCT
GTCCTCCGGGCAAGTACAAATACGGCGTTGGGGACGACAAGTGTCAGCCGTGTCCGGCG
CACAGCAAAGCGCCCGACCAGGGGATGTCCGAGTGCAGGTGCAACACCGGATACTACAG
GTCGCCCAAGGACCCAAAATCGGTGCCGTGCACGCAACCGCCGTCAGCGCCGCAAAACT
TGACAGTCAACTTTGTGGACCAGTCGACTGTGACCCTGTCGTGGAACCCACCGAACTTCC
TCGGTGGCAGGACCGATATCGTGTACAGAGTGACATGCGACATGTGCGGTCCATCCGTTG
TGTTCATGCCCAACAACGAGGTGTTCAACGATACCAAAATAACGATCAGCGGTCTGGGT
CCGGTCACCACGTACAAGTTTCACGTATGGGCCGAAAACGGTGTCAGTAATCTAACGTCG
TCCGAAAACCGGCAATTCGTCGACATAGCCGTGACCACTACAGAAGCTTCCGTTAAGTCT
GCGTCCGTCAACAATGTCCGTGTCATGTTGGTAAAGGCGTCCGAGATCACGCTATCTTGG
GATCCGCCCATGGCGAGCTTCTTCGATTCTGGCGATGACGACGCTGCCGTCGAAGTTTAC
GAAGTGAAGTTCTATCCCCGAGGTGACGAGTCAAACGTCAGCAACAAACTTACCGCCGA
CAGGCACATGGTGTTTACGGCACTCAGGCCCAAGACGGACTACGGATTCCAAGTGCGAG
CGAAGACCGCTCACGGCTGGGGCGAGTACAGTCCGACTATTTACAAAACGACGGGGCAA
TTGCTCGGAAGCGGTAAGAACACACAACAACAAGTCCAAAGAAAAAACGGGATTAAAG
ATGATACGGCCTACATCGGTGACGAAGACAACATGGAAGTCCGAATAATCGCAGGTGCG
ACGGTCGCGGTAGTGGTGGTCTTAGTAGTTGTCATCATCATGACGGTATTGTTCTTAGGA
GGAGACCGGTACATATACGGGCATACCTGCTTGATAACTCTGACAGTGCTGCATGTGATG
CTTTCACTTATTGGTTACTTGACTTGA
mpEPH receptor with CD55 signal peptide (1-102) and CD55 GPI anchor (1960-2046)
(RNA)
AUGAUAAGAGGCCGCGCCCCACGCACCCGCCCUAGUCCCCCUCCUCCUUUGUUGCCAC
UUCUUUCUCUGUCCCUUCUCCUCCUCUCUCCUACUGUGAGGGGUCACAAGUAUCACCA
ACACCACUGCGACGACAGCAGCCAUCACCACCAUCGGCACAACCACCAUCAUCACCUC
AACCGUCACCACCGAUGCUGUCUCGCCGCGGUGUUUCUGCUGCUGGCCGUCGCGAUCG
UCAGCCCCGGACGAGCCGAACACGUUGUACUUUUGGACACCACGACAGAGCCUAGUCU
ACGGUGGACUACGUACCCUUACGGACCGGACGCCAACGCAGCAGGGUGGGUCGAAGAG
AGCUACAUAAAUUUUGAAAAGGGCAUCAACUGGCGGUCGUACGUCGUGUGCGAUGUC
ACCAAGCAAAACGUCAACAAUUGGGUGUGGACGCCUUUCAUAGAGCGCGGGCUGGCC
AAUCUCAUUUACAUCGAAGUCAAGUUUACCAUCCGAGAUUGUUCGCUGUUCCCGGGG
UACGCGCUGUCAUGCAAAGAAACGUUUUCACUACUCUACUAUGAGUUUGACGUGGCC
ACCAGGGAACCACCGCCUUGGGAACCUGACAGCUAUAAAUCUGUUGGUCGGAUAGCU
GCUGGUGAAGGGAGGUUCAACGCUAAUAACGAAGUGGUGAUCAAUACUGAGACAAAA
GCUGUGAAAGUUACCAAAAAAGGAGUGUACUUUGCCUUCAGGGACCAGGGCGCAUGU
AUUUCCAUCAUGGCAGUCAAGGUGUAUUACAUCGUGUGCCCAGAAGUUGUGAUAAAC
UUUGCAAACUUCUCUGCUACUCCAACUGCUAGAGAACUUACCCAAAUCGAACAUGCCA
CCGGCAAAUGUGUUGAUAAUGCUGAAGUGGUAGGAGGUGGUGCUCCAACUUACCUGU
GCAAAGGAGAUGGAAAAUGGUAUCUACCGUCAGGUGGAUGCAAGUGCAAAGCUGGUU
UUGAAGCAGACAUAGAAGCGCAAACUUGUAUAAUCUGUCCUCCGGGCAAGUACAAAU
ACGGCGUUGGGGACGACAAGUGUCAGCCGUGUCCGGCGCACAGCAAAGCGCCCGACCA
GGGGAUGUCCGAGUGCAGGUGCAACACCGGAUACUACAGGUCGCCCAAGGACCCAAAA
UCGGUGCCGUGCACGCAACCGCCGUCAGCGCCGCAAAACUUGACAGUCAACUUUGUGG
ACCAGUCGACUGUGACCCUGUCGUGGAACCCACCGAACUUCCUCGGUGGCAGGACCGA
UAUCGUGUACAGAGUGACAUGCGACAUGUGCGGUCCAUCCGUUGUGUUCAUGCCCAA
CAACGAGGUGUUCAACGAUACCAAAAUAACGAUCAGCGGUCUGGGUCCGGUCACCACG
UACAAGUUUCACGUAUGGGCCGAAAACGGUGUCAGUAAUCUAACGUCGUCCGAAAAC
CGGCAAUUCGUCGACAUAGCCGUGACCACUACAGAAGCUUCCGUUAAGUCUGCGUCCG
UCAACAAUGUCCGUGUCAUGUUGGUAAAGGCGUCCGAGAUCACGCUAUCUUGGGAUC
CGCCCAUGGCGAGCUUCUUCGAUUCUGGCGAUGACGACGCUGCCGUCGAAGUUUACGA
AGUGAAGUUCUAUCCCCGAGGUGACGAGUCAAACGUCAGCAACAAACUUACCGCCGAC
AGGCACAUGGUGUUUACGGCACUCAGGCCCAAGACGGACUACGGAUUCCAAGUGCGA
GCGAAGACCGCUCACGGCUGGGGCGAGUACAGUCCGACUAUUUACAAAACGACGGGGC
AAUUGCUCGGAAGCGGUAAGAACACACAACAACAAGUCCAAAGAAAAAACGGGAUUA
AAGAUGAUACGGCCUACAUCGGUGACGAAGACAACAUGGAAGUCCGAAUAAUCGCAG
GUGCGACGGUCGCGGUAGUGGUGGUCUUAGUAGUUGUCAUCAUCAUGACGGUAUUGU
UCUUAGGAGGAGACCGGUACAUAUACGGGCAUACCUGCUUGAUAACUCUGACAGUGC
UGCAUGUGAUGCUUUCACUUAUUGGUUACUUGACUUGA

This mpEPH receptor can be used in conjunction with TuYV to deliver a nucleic acid of interest to a cell transiently expressing the mpEPH receptor. The mpEPH receptor can be used in conjunction with a TuYV of SEQ ID NO: 6.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention and are not to be construed as limiting in any way the remainder of the disclosure.

It is to be understood that while the disclosure has been described in conjunction with the preferred specific embodiments thereof, that the foregoing description and the examples that follow are intended to illustrate and not limit the scope of the disclosure. It will be understood by those skilled in the art that various changes may be made, and equivalents may be substituted without departing from the scope of the disclosure, and further that other aspects, advantages and modifications will be apparent to those skilled in the art to which the disclosure pertains. In addition to the embodiments described herein, the present disclosure contemplates and claims those inventions resulting from the combination of features of the disclosure cited herein and those of the cited prior art references which complement the features of the present disclosure. Similarly, it will be appreciated that any described material, feature, or article may be used in combination with any other material, feature, or article, and such combinations are considered within the scope of this disclosure.

The disclosures of each patent, patent application, and publication cited or described herein are hereby incorporated herein by reference, each in its entirety, for all purposes.

1. A modified plant virus comprising a plant virus nucleotide sequence and a nucleotide sequence capable of exhibiting a therapeutic effect in a mammalian cell, wherein the virus is capable of transfecting a mammalian cell, wherein the mammalian cell is modified to express a receptor for the modified plant virus.

2. The modified plant virus of claim 1, wherein the nucleotide sequence capable of exhibiting a therapeutic effect is a mammalian gene.

3. The modified plant virus of claim 1, wherein the nucleotide sequence capable of exhibiting a therapeutic effect is a nuclease.

4. The modified plant virus of claim 3, wherein the nuclease is a CRISPR/cas construct, TALENS, or ZFNs.

5. The modified plant virus of claim 1, wherein the plant virus nucleotide sequence comprises one or more of ORF I, ORF II, ORF III, ORF IV, ORF V, ORF VI, ORF VII and ORF VIII.

6. The modified plant virus of claim 1, wherein the plant virus is Cauliflower Mosaic virus, Tomato Yellow Leaf Curl Virus (TYLCV) or Turnip Yellow Virus (TuYV).

7. The modified plant virus of claim 1, wherein the nucleotide sequence capable of exhibiting a therapeutic effect in a mammalian cell is present within the plant virus nucleotide sequence.

8. The modified plant virus of claim 1, wherein the plant virus comprises a first plant virus sequence and a second plant virus sequence and wherein the nucleotide sequence capable of exhibiting a therapeutic effect in a mammalian cell is present within the second plant virus nucleotide sequence.

9. The modified plant virus of claim 1, wherein the plant virus nucleotide sequence comprises the nucleotide sequence of SEQ ID NO: 1-6 or a fragment thereof capable of transfecting a mammalian cell that has been modified to express a receptor for the modified plant virus.

10. The modified plant virus of claim 9, wherein the nucleotide sequence capable of exhibiting a therapeutic effect in a mammalian cell is present within the plant virus sequence of SEQ ID NO: 1-3, 5 or 6.

11. The modified plant virus of claim 9, wherein the nucleotide sequence capable of exhibiting a therapeutic effect in a mammalian cell is present within the plant virus sequence of SEQ ID NO: 2 at nucleotides 3424-6101.

12. The modified plant virus of claim 9, wherein the nucleotide sequence capable of exhibiting a therapeutic effect in a mammalian cell is present within the plant virus sequence of SEQ ID NO: 3 at nucleotides 1596-6101.

13. The modified plant virus of claim 9, wherein the nucleotide sequence capable of exhibiting a therapeutic effect in a mammalian cell is present within the plant virus sequence of SEQ ID NO: 5 at position 875.

14. The modified plant virus of claim 9, wherein the nucleotide sequence capable of exhibiting a therapeutic effect in a mammalian cell is present within the plant virus sequence of SEQ ID NO: 6 at position 4946.

15. A pharmaceutical composition comprising the modified plant virus of claim 1 and a pharmaceutically acceptable carrier.

16. A plant cell comprising the modified plant virus of claim 1.

17.-71. (canceled)


SEQUENCE LISTING (15 sequences)

SEQ ID NO: 1 - DNA, 10995 bp, synthetic construct
  misc_feature 1..10995: note = synthetic Polynucleotide
  source 1..10995: mol_type = other DNA
ggtatcagagccatgaataggtctatgaccaaaactcaagaggataaaacctcaccaaaataccaaagagttcttaactctaaagataaaagatctttcaagatcaaaaatagttccctcacaccggtgaccgacaggtttaccaccgtaaggtttcagaacaacatcgaatgcgtttacgccaacttcgactctcagctcaagtcgtcgtacgatggtagatctaaaaagatcaagaatctaagccttaaaaatcttagatgctacgaagccttcctcaggaagtaccttctggaacaataaatctctctgagaatagtactctaacgagtatccacagataaaataatcttctgtgttgagatggatttgtatccagaggaaaatacccaaagcgagcaatcgcataattctgaaaataatatgcaaatatttaaatcagaaaattcggatggattctcttccgatctaatgatctcaaacgatcaattaaaaaatatctctaaaacccaattgacattggagaaagaaaagatatttaaaatgcctaacgttttatctcaagttatgaaaaaagcgtttagcaggaaaaacgagatactttactgcgtctcgacaaaagaattatcggtggacattcacgatgccacaggtaaggtatatcttcctttaatcactaaagaggagataaataaaagactttccagcttaaaacctgaagtcagaagaaccatgtccatggtccacttgggcgcggttaaaatattgcttaaagctcaatttcgaaatgggattgataccccaatcaaaattgctttaatcgatgatagaatcaattctagaaaagattgccttctcggtgcagccaaaggtaatctagcatacggtaagtttatgtttactgtataccccaagtttggaataagccttaatacccaaagacttaaccaaaccctaagccttattcatgattttgagaataaaaatcttatgaataaaggtgataaagttatgaccataacctatattgtaggatatgcattaaccaatagtcatcatagcatagattatcaatcgaatgctacaattgaactagaagacgtatttcaagaaattggaaatgtccagcaatctgagttctgtacaatacagaatgatgaatgcaattgggccattgatatagcccaaaacaaagccttattaggagctaaaaccaaatcccaaattggtaatagtcttcaaataggaaacagtgcttcatcctctaatactgaaaatgaattagctagggtaagccagaacatagatcttttaaagaataaattaaaagaaatctgtggagaataatatgagcattacgggtcaaccgcatgtttataaaaaagatactattattagactaaaaccattgtctcttaatagtaataatagaagttatgtttttagttcctcaaaagggaatattcaaaatataattaatcatcttaacaacctcaatgagattgtaggaagaagcttactcggaatatggaagatcaactcatacttcggactaagcaaagacccttcggagtccaaatcaaaaaacccgtcagtttttaatactgcaaaaaccatttttaagagtgggggggttgattactcgagccaattaaaggaaataaaatcccttttagaagctcaaaatactagaattaaaaatctagaaaaagcagttcaagccttagataataagattgaaccagagcccttaactaaagaagaagttaaagaacttaaagaatcgattaactcgatcaaagaaggattaaagaatattattggctgaaatggctaatcttaatcaaatccaaaaagaagtctctgagatcctcagtgaccaaaaatccatgaaagcggatataaaagctatcttagaattattaggatcccaaaatcctattaaagaaagcttagaaaccgttgcagcgaaaatcgttaatgacttaaccaagctcatcaatgattgtccttgtaacaaagagatattagaagccttaggcaatcagcctaaagagcaactaattgaacaacctaaagaaaaaggcaaaggccttaatctaggaaaatactcttaccccaattacggagtaggaaatgaagaattaggatcctctggaaaccctaaagctttaacctggcccttcaaagctccagcaggatggccgaatcaattttagatggatcatctacttctgaagactcagactcagactgagcaggtgatgaacgtcaccaatcccaattcaatctacatcaagggaagactctacttcaaaggatacaagaagatagagcttcattgtttcgtagacacgggagcaagcttatgcatagcatccaagttcgtcattccagaagaacattgggtcaatgcagaaagaccaataatggtcaaaatagcagatggaagctcaatcaccatcagcaaagtctgcaaagacatagacttgatcatagccggcgagatattcaaaattcccaccgtctatcagcaagaaagtggtatcgatttcataatcggcaacaacttctgtcagctgtatgaaccattcattcagtttacagatagagttatcttcacaaagaacaagtcctatcctgttcatattgcgaagctaacaagagcagtgcgagtaggcaccgaaggatttcttgaatcaatgaagaaacgttcaaagactcaacaaccagagccagtgaacatttctacaaacaagatagaaaatccaaaagaagaaattgctattctttcagaggggaggaggttagcagaagaaaaactcttcatcactcaacaaagaatgcaaaaaatcgaagaactacttgagaaagtatgttcagaaaatccattagatcctaacaagactaagcaatggatgaaagcttcaatcaagctcagcgacccaagcaaagctatcaaggttaaacccatgaagtatagcccaatggatcgtgaagaatttgacaagcaaatcaaagagttactggacctaaaagtcattaagcccagtaaaagccctcacatggcaccagccttcttggtcaataatgaagccgagaagcgaagaggaaagaagcgtatggtagtcaactacaaggctatgaacaaagccaccataggagacgcatacaatcttcccaataaagacgagttactgacacttattcgaggaaagaagatcttctcttccttcgactgcaagtcaggtttctggcaggttctgctagatcaagaatcaagacctctaacggcattcacatgtccccaaggtcactacgaatggaatgtggtccctttcggcctaaagcaggctccatccatattccaaagacacatggacgaagcatttcgtgtgttcaggaagttctgttgcgtgtatgtcgacgacattctcgtattcagtaacaacgaagaagatcacctacttcacgtagcaatgatcttacaaaagtgcaatcaacatggaatcatcctttccaagaagaaagcacaactcttcaaaaagaagataaacttccttggtctagaaatagatgaaggaacacataagcctcaaggacatatcttggaacacatcaacaaattcccagatacccttgaagacaagaagcaacttcagagattcttaggcatactaacatatgcctcggattatatcccgaagctagctcaaatcagaaagcctctgcaagccaagcttaaggagaacgttccatggaaatggacgaaagaggacaccctctacatgcaaaaggtgaagaaaaatctgcaaggatttcctccactacatcatcccttaccagaagagaagctgatcatcgagaccgacgcatcagacgactactggggaggtatgttaaaagctatcaaaattaacgaaggtactaatactgagttaatttgcagatacgcatctggaagctttaaagctgcagaaaagaattaccacagcaatgacaaagaaacattggcggtaataaatactataaagaaattcagtatttatctaactcctgttcatttcttaatcaggacagataatacgcatttcaagagttttgttaaccttaattacaaaggtgattcaaaacttggaagaaacatcagatggcaagcatggcttagccactattcatttgatgttgaacacattaaaggaaccgacaaccactttgcggacttcctttcaagagaattcaataaggttaattcctaattgaaatccgaagataagattcccacacacttgtggctgatatcaaaaggctactgcctatataaacacatctctggagactgagaaaatcagacctccaagcatggagaacatagaaaaactcctcatgcaagagaaaatactaatgctagagctcgatctagtaagagcaaaaataagcttagcaagagctaacggctcttcgcaacaaggagacctccctctccaccgtgaaacaccggtaaaagaagaagcagttcactctgcactggctacttttacgccatctcaagtaaaagctattccagagcaaacggctcctggtaaagaatcaacaaatccgttgatggctagtatcttgccaaaagatatgaacccagttcagactgggatgaggcttacagtgccaggggactttttacgtcctcatcagggaattccaatcccacgaaaatctgagcttagcagcacagttgctcctctcagggcagaatcgggtattcaacaccctcatatcaactactacgttgtgtataacggtccacacgccggtatatacgatgactggggttgtacaaaggcggcaacaaacggtgttcccggagttgcacataagaagtttgccactattacagaggcaagagcagcagctgacgcatatacaacaagtcagcaaacagataggttgaacttcatccccaaaggagaagctcaactcaagcccaaaagctttgccaaggctctcaccagcccaccaaagcaaaaagcccactggctcacgctaggaaccaaaaagcccagcagtgatccagccccaaaagagatctccttcgacccggagatcacaatggacgacttcctctatctctatgatctagtcaggaagttcgacggagaaggtgacgataccatgttcaccactgataatgagaagattagcctcttcaatttcagaaagaacgctaacccacagatggttagagaggcctacgcagcaggtctcatcaagacgatctacccgagcaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaagacaagtaatagagatcggagtctctaagaaattagttcctacagagtctaaggccatggagtcaaagattcaaattgaggacctaacagaactcgccgtgaagactggcgaacagttcatacagagtctcttaagactcaatgacaagaagaaaatcttcgtcaacatggtggagcacgacactctgatctactccaaaaatatcaaagaaacagtctcagaagaccaaagggcaattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtgagactttccaacaaagggtaatatcgggaaacctcctcggattccattgcccagctatctgtcactttattgtgaagatagtggaaaaggaaggtggcacctaccaatgccatcattgcgataaaggaaaggctatcgttaaagatgcctctgccgacagtggtcctaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacacaaaaaaaaacacaaaaaaatcaccagtctctctctacaaatctatctctctctattttctccataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtggtgaatataaaccagacacgagacgactaaacctggagcccagacgccgtttgaagctagaagtaccgcttaggcaggaggccgttagggaaaagatgctaaggcagggttggttacgttgactcccccgtaggtttggtttaaatatcatgaagtggacggaaggaaggaggaagacaaggaaggataaggttgcaggccctgtgcaaggtaagaagatggaaatttgatagaggtacgttactatactcatactatacgctaagggaatgcttgtatttaccctatataccctaataaccccttatcgatttaaagaaataatccgcataagcccccgcttaaaaaatt

SEQ ID NO: 2 - DNA, 12971 bp, synthetic construct
  misc_feature 1..12971: note = synthetic Polynucleotide
  source 1..12971: mol_type = other DNA
ggtatcagagccatgaataggtctatgaccaaaactcaagaggataaaacctcaccaaaataccaaagagttcttaactctaaagataaaagatctttcaagatcaaaaatagttccctcacaccggtgaccgacaggtttaccaccgtaaggtttcagaacaacatcgaatgcgtttacgccaacttcgactctcagctcaagtcgtcgtacgatggtagatctaaaaagatcaagaatctaagccttaaaaatcttagatgctacgaagccttcctcaggaagtaccttctggaacaataaatctctctgagaatagtactctaacgagtatccacagataaaataatcttctgtgttgagatggatttgtatccagaggaaaatacccaaagcgagcaatcgcataattctgaaaataatatgcaaatatttaaatcagaaaattcggatggattctcttccgatctaatgatctcaaacgatcaattaaaaaatatctctaaaacccaattgacattggagaaagaaaagatatttaaaatgcctaacgttttatctcaagttatgaaaaaagcgtttagcaggaaaaacgagatactttactgcgtctcgacaaaagaattatcggtggacattcacgatgccacaggtaaggtatatcttcctttaatcactaaagaggagataaataaaagactttccagcttaaaacctgaagtcagaagaaccatgtccatggtccacttgggcgcggttaaaatattgcttaaagctcaatttcgaaatgggattgataccccaatcaaaattgctttaatcgatgatagaatcaattctagaaaagattgccttctcggtgcagccaaaggtaatctagcatacggtaagtttatgtttactgtataccccaagtttggaataagccttaatacccaaagacttaaccaaaccctaagccttattcatgattttgagaataaaaatcttatgaataaaggtgataaagttatgaccataacctatattgtaggatatgcattaaccaatagtcatcatagcatagattatcaatcgaatgctacaattgaactagaagacgtatttcaagaaattggaaatgtccagcaatctgagttctgtacaatacagaatgatgaatgcaattgggccattgatatagcccaaaacaaagccttattaggagctaaaaccaaatcccaaattggtaatagtcttcaaataggaaacagtgcttcatcctctaatactgaaaatgaattagctagggtaagccagaacatagatcttttaaagaataaattaaaagaaatctgtggagaataatatgagcattacgggtcaaccgcatgtttataaaaaagatactattattagactaaaaccattgtctcttaatagtaataatagaagttatgtttttagttcctcaaaagggaatattcaaaatataattaatcatcttaacaacctcaatgagattgtaggaagaagcttactcggaatatggaagatcaactcatacttcggactaagcaaagacccttcggagtccaaatcaaaaaacccgtcagtttttaatactgcaaaaaccatttttaagagtgggggggttgattactcgagccaattaaaggaaataaaatcccttttagaagctcaaaatactagaattaaaaatctagaaaaagcagttcaagccttagataataagattgaaccagagcccttaactaaagaagaagttaaagaacttaaagaatcgattaactcgatcaaagaaggattaaagaatattattggctgaaatggctaatcttaatcaaatccaaaaagaagtctctgagatcctcagtgaccaaaaatccatgaaagcggatataaaagctatcttagaattattaggatcccaaaatcctattaaagaaagcttagaaaccgttgcagcgaaaatcgttaatgacttaaccaagctcatcaatgattgtccttgtaacaaagagatattagaagccttaggcaatcagcctaaagagcaactaattgaacaacctaaagaaaaaggcaaaggccttaatctaggaaaatactcttaccccaattacggagtaggaaatgaagaattaggatcctctggaaaccctaaagctttaacctggcccttcaaagctccagcaggatggccgaatcaattttagatggatcatctacttctgaagactcagactcagactgagcaggtgatgaacgtcaccaatcccaattcaatctacatcaagggaagactctacttcaaaggatacaagaagatagagcttcattgtttcgtagacacgggagcaagcttatgcatagcatccaagttcgtcattccagaagaacattgggtcaatgcagaaagaccaataatggtcaaaatagcagatggaagctcaatcaccatcagcaaagtctgcaaagacatagacttgatcatagccggcgagatattcaaaattcccaccgtctatcagcaagaaagtggtatcgatttcataatcggcaacaacttctgtcagctgtatgaaccattcattcagtttacagatagagttatcttcacaaagaacaagtcctatcctgttcatattgcgaagctaacaagagcagtgcgagtaggcaccgaaggatttcttgaatcaatgaagaaacgttcaaagactcaacaaccagagccagtgaacatttctacaaacaagatagaaaatccaaaagaagaaattgctattctttcagaggggaggaggttagcagaagaaaaactcttcatcactcaacaaagaatgcaaaaaatcgaagaactacttgagaaagtatgttcagaaaatccattagatcctaacaagactaagcaatggatgaaagcttcaatcaagctcagcgacccaagcaaagctatcaaggttaaacccatgaagtatagcccaatggatcgtgaagaatttgacaagcaaatcaaagagttactggacctaaaagtcattaagcccagtaaaagccctcacatggcaccagccttcttggtcaataatgaagccgagaagcgaagaggaaagaagcgtatggtagtcaactacaaggctatgaacaaagccaccataggagacgcatacaatcttcccaataaagacgagttactgacacttattcgaggaaagaagatcttctcttccttcgactgcaagtcaggtttctggcaggttctgctagatcaagaatcaagacctctaacggcattcacatgtccccaaggtcactacgaatggaatgtggtccctttcggcctaaagcaggctccatccatattccaaagacacatggacgaagcatttcgtgtgttcaggaagttctgttgcgtgtatgtcgacccactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggccctcgacctgcagcccaagctgtaccccctcgaaactgacaggagaaccaccatgggcccaaagaagaagagaaaggtttcgaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgcgaccagcttgatatcgaattgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctacgtattaactagctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtgagactttccaacaaagggtaatatcgggaaacctcctcggattccattgcccagctatctgtcactttattgtgaagatagtggaaaaggaaggtggcacctaccaatgccatcattgcgataaaggaaaggctatcgttaaagatgcctctgccgacagtggtcctaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacacaaaaaaaaacacaaaaaaatcaccagtctctctctacaaatctatctctctctattttctccataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtggtgaatataaaccagacacgagacgactaaacctggagcccagacgccgtttgaagctagaagtaccgcttaggcaggaggccgttagggaaaagatgctaaggcagggttggttacgttgactcccccgtaggtttggtttaaatatcatgaagtggacggaaggaaggaggaagacaaggaaggataaggttgcaggccctgtgcaaggtaagaagatggaaatttgatagaggtacgttactatactcatactatacgctaagggaatgcttgtatttaccctatataccctaataaccccttatcgatttaaagaaataatccgcataagcccccgcttaaaaaatt

SEQ ID NO: 3 - DNA, 11250 bp, synthetic construct
  misc_feature 1..11250: note = synthetic Polynucleotide
  source 1..11250: mol_type = other DNA
ggtatcagagccatgaataggtctatgaccaaaactcaagaggataaaacctcaccaaaataccaaagagttcttaactctaaagataaaagatctttcaagatcaaaaatagttccctcacaccggtgaccgacaggtttaccaccgtaaggtttcagaacaacatcgaatgcgtttacgccaacttcgactctcagctcaagtcgtcgtacgatggtagatctaaaaagatcaagaatctaagccttaaaaatcttagatgctacgaagccttcctcaggaagtaccttctggaacaataaatctctctgagaatagtactctaacgagtatccacagataaaataatcttctgtgttgagatggatttgtatccagaggaaaatacccaaagcgagcaatcgcataattctgaaaataatatgcaaatatttaaatcagaaaattcggatggattctcttccgatctaatgatctcaaacgatcaattaaaaaatatctctaaaacccaattgacattggagaaagaaaagatatttaaaatgcctaacgttttatctcaagttatgaaaaaagcgtttagcaggaaaaacgagatactttactgcgtctcgacaaaagaattatcggtggacattcacgatgccacaggtaaggtatatcttcctttaatcactaaagaggagataaataaaagactttccagcttaaaacctgaagtcagaagaaccatgtccatggtccacttgggcgcggttaaaatattgcttaaagctcaatttcgaaatgggattgataccccaatcaaaattgctttaatcgatgatagaatcaattctagaaaagattgccttctcggtgcagccaaaggtaatctagcatacggtaagtttatgtttactgtataccccaagtttggaataagccttaatacccaaagacttaaccaaaccctaagccttattcatgattttgagaataaaaatcttatgaataaaggtgataaagttatgaccataacctatattgtaggatatgcattaaccaatagtcatcatagcatagattatcaatcgaatgctacaattgaactagaagacgtatttcaagaaattggaaatgtccagcaatctgagttctgtacaatacagaatgatgaatgcaattgggccattgatatagcccaaaacaaagccttattaggagctaaaaccaaatcccaaattggtaatagtcttcaaataggaaacagtgcttcatcctctaatactgaaaatgaattagctagggtaagccagaacatagatcttttaaagaataaattaaaagaaatctgtggagaataatatgagcattacgggtcaaccgcatgtttataaaaaagatactattattagactaaaaccattgtctcttaatagtaataatagaagttatgtttttagttcctcaaaagggaatattcaaaatataattaatcatcttaacaacctcaatgagattgtaggaagaagcttactcggaatatggaagatcaactcatacttcggactaagcaaagacccttcggagtccaaatcaaaaaacccgtcagtttttaatactgcaaaaaccatttttaagagtgggggggttgattactcgacccactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattctgcagtcgacggtaccgcgggccctcgacctgcagcccaagctgtaccccctcgaaactgacaggagaaccaccatgggcccaaagaagaagagaaaggtttcgaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagccattaacgcgcgaccagcttgatatcgaattgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctacgtattaaattgctattctttcagaggggaggaggttagcagaagaaaaactcttcatcactcaacaactagctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtgagactttccaacaaagggtaatatcgggaaacctcctcggattccattgcccagctatctgtcactttattgtgaagatagtggaaaaggaaggtggcacctaccaatgccatcattgcgataaaggaaaggctatcgttaaagatgcctctgccgacagtggtcctaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacacaaaaaaaaacacaaaaaaatcaccagtctctctctacaaatctatctctctctattttctccataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtggtgaatataaaccagacacgagacgactaaacctggagcccagacgccgtttgaagctagaagtaccgcttaggcaggaggccgttagggaaaagatgctaaggcagggttggttacgttgactcccccgtaggtttggtttaaatatcatgaagtggacggaaggaaggaggaagacaaggaaggataaggttgcaggccctgtgcaaggtaagaagatggaaatttgatagaggtacgttactatactcatactatacgctaagggaatgcttgtatttaccctatataccctaataaccccttatcgatttaaagaaataatccgcataagcccccgcttaaaaaatt

SEQ ID NO: 4 - DNA, 2787 bp, synthetic construct
  misc_feature 1..2787: note = synthetic Polynucleotide
  source 1..2787: mol_type = other DNA
gttgaaatgaatcggtgtccctcaaagctctatggcaatcggtgtatcggtgtcttacttatacttggacacctaatggctatttggtaatttcataaatgttcatttcaattcaaaattcaaaattcaaaaatcaaatcattaaagcggccatccgtataatattaccggatggccgcgccttttccttttatgtggtccccacgagggttacacagatgttattgtcaaccaatcaaattgcattctcaaacgttagataagtgttcatttgtctttatatacttggtccccaagttttttgtcttgcaatatgtgggacccacttcttaatgaatttcctgaatctgttcacggatttcgttgtatgttagctattaaatatttgcagtccgttgaggaaacttacgagcccaatacattgggccacgatttaattagggatcttatatctgttgtaagggcccgtgactatgtcgaagcgaccaggcgatataatcatttccacgcccgtctcgaaggttcgccgaaggctgaacttcgacagcccatacagcagccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcaacgggatgatattaagcatactggtattgttcgttgtgttagtgatgttactcgtggatctggaattactcacagagtgggtaagaggttctgtgttaaatcgatatattttttaggtaaagtctggatggatgaaaatatcaagaagcagaatcacactaatcaggtcatgttcttcttggtccgtgatagaaggccctatggaaacagcccaatggattttggacaggtttttaatatgttcgataatgagcccagtaccgcaaccgtgaagaatgatttgcgtgataggtttcaagtgatgaggaaatttcatgctacagttattggtgggccctctggaatgaaggaacaggcattagttaagagattttttaaaattaacagtcatgtaactttatttatattcattcaggaggcagcaaagtacgagaaccatactgaaaacgccttgttattgtatatggcatgtacgcatgcctctaatccagtgtatgcaactatgaaaatacgcatctatttctatgattcaatatcaaattaataaaatttatattttatatcatgagtttctgttacatttattgtgttttcaagtacatcatacaatacatgatcaactgctctgattacattgttaatggaaattacaccaagactatctaaatacttaagaacttcatatctaaatactcttaagaaatgaccagtctgaggctgtaatgtcgtccaaattcggaagtcgagaaaacatttgtgaatccccattaccttcctgatgttgtggttgaatcttatctgaatggaaatgatgtcgtggttcattagaaatggcctctggctgtgttctgttatcttgaaatagaggggattgtttatctcccagataaaaacgccattctctgcctgaggagcagtgatgagttcccctgtgcgtgaatccatgattattgcagttgaggtggaggtagtatgagcagccacagtctaggtctacacgcttacgccttattggtttcttcttggctatcttgtgttggaccttgattgatacttgcgaacagtggctcgtagagggtgacgaaggttgcattcttgagagcccaatttttcaaggatatgtttttttcttcgtctagatattccctatatgaggaggtaggtcctggattgcagaggaagatagtgggaattccccctttaatttgaatgggcttcccgtactttgtgttgctttgccagtccctctgggcccccatgaattccttgaagtgctttaaataatgcgggtctacgtcatcaatgacgttgtaccacgcatcattactgtacacctttgggcttaggtctagatgtccacataaataattatgtgggcctagagacctggcccacattgttttgcctgttctgctatcaccctcaatgacaatacttatgggtctccatggccgcgcagcggaatacacgacgttctcggcgacccactcttcaagttcatctggaacttgattaaaagaagaagaaagaaatggagaaacataaacttctaaaggaggactaaaaatcctatctaaatttgaacttaaattatgaaattgtaaaatatagtcctttggggccttctcttttaatatattgagggcctcggatttactgcctgaattgagtgcttcggcatatgcgtcgttggcagattgctgacctcctctagctgatctgccatcgatttgggaaactccaaaatcaatgaagtttccgtctttctccacgtaggtcttgacatctgttgagctcttagctgcctgaatgttcggatggaaatgtgctgacctgtttggggataccaagtcgaagaaccgttggttcttacattggtatttgccttcgaattggataagcacatggagatgtggttccccattctcgtggagttctttgcaaactttgatgtattttttatttgttggggtttctagtttttttaattgggaaagtgcttcctctttagagagagaacaattgggatatgttaggaaataatttttggcatatattttaaataaacgaggcat

SEQ ID NO: 5 - DNA, 2122 bp, synthetic construct
  misc_feature 1..2122: note = synthetic Polynucleotide
  source 1..2122: mol_type = other DNA
cttagctgcctgaatgttcggatggaaatgtgctgacctgtttggggataccaagtcgaagaaccgttggttcttacattggtatttgccttcgaattggataagcacatggagatgtggttccccattctcgtggagttctttgcaaactttgatgtattttttatttgttggggtttctagtttttttaattgggaaagtgcttcctctttagagagagaacaattgggatatgttaggaaataatttttggcatatattttaaataaacgaggcatgttgaaatgaatcggtgtccctcaaagctctatggcaatcggtgtatcggtgtcttacttatacttggacacctaatggctatttggtaatttcataaatgttcatttcaattcaaaattcaaaattcaaaaatcaaatcattaaagcggccatccgtataatattaccggatggccgcgccttttccttttatgtggtccccacgagggttacacagatgttattgtcaaccaatcaaattgcattctcaaacgttagataagtgttcatttgtctttatatacttggtccccaagttttttgtcttgcaatatgtgggacccacttcttaatgaatttcctgaatctgttcacggatttcgttgtatgttagctattaaatatttgcagtccgttgaggaaacttacgagcccaatacattgggccacgatttaattagggatcttatatctgttgtaagggcccgtgactatgtcgaagcgaccaggcgatataatcatttccacgcccgtctcgaaggttcgccgaaggctgaacttcgacagcccatacagcagccgtgctgctgtccccattgtccaaggcacaaacaaggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 6 - RNA, 5581 nt, synthetic construct
  misc_feature 1..5581: note = synthetic Polynucleotide
  source 1..5581: mol_type = other RNA
  modified_base 19: mod_base = OTHER; note = Uracil
  modified_base 22: mod_base = OTHER; note = Uracil
  modified_base 26: mod_base = OTHER; note = Uracil
  modified_base 27: mod_base = OTHER; note = Uracil
  modified_base 30: mod_base = OTHER; note = Uracil
  modified_base 35: mod_base = OTHER; note = Uracil
  modified_base 36: mod_base = OTHER; note = Uracil
  modified_base 37: mod_base = OTHER; note = Uracil
  modified_base 39: mod_base = OTHER; note = Uracil
  modified_base 43: mod_base = OTHER; note = Uracil
  modified_base 49: mod_base = OTHER; note = Uracil
  modified_base 53: mod_base = OTHER; note = Uracil
  modified_base 54: mod_base = OTHER; note = Uracil
  modified_base 55: mod_base = OTHER; note = Uracil
  modified_base 61: mod_base = OTHER; note = Uracil
  modified_base 63: mod_base = OTHER; note = Uracil
  modified_base 69: mod_base = OTHER; note = Uracil
  modified_base 78: mod_base = OTHER; note = Uracil
  modified_base 83: mod_base = OTHER; note = Uracil
  modified_base 87: mod_base = OTHER; note = Uracil
  modified_base 102: mod_base = OTHER; note = Uracil
  modified_base 107: mod_base = OTHER; note = Uracil
  modified_base 108: mod_base = OTHER; note = Uracil
  modified_base 109: mod_base = OTHER; note = Uracil
  modified_base 111: mod_base = OTHER; note = Uracil
  modified_base 112: mod_base = OTHER; note = Uracil
  modified_base 113: mod_base = OTHER; note = Uracil
  modified_base 114: mod_base = OTHER; note = Uracil
  modified_base 121: mod_base = OTHER; note = Uracil
  modified_base 123: mod_base = OTHER; note = Uracil
  modified_base 125: mod_base = OTHER; note = Uracil
  modified_base 126: mod_base = OTHER; note = Uracil
  modified_base 129: mod_base = OTHER; note = Uracil
  modified_base 130: mod_base = OTHER; note = Uracil
  modified_base 135: mod_base = OTHER; note = Uracil
  modified_base 143: mod_base = OTHER; note = Uracil
  modified_base 144: mod_base = OTHER; note = Uracil
  modified_base 145: mod_base = OTHER; note = Uracil
  modified_base 147: mod_base = OTHER; note = Uracil
  modified_base 156: mod_base = OTHER; note = Uracil
  modified_base 172: mod_base = OTHER; note = Uracil
  modified_base 176: mod_base = OTHER; note = Uracil
  modified_base 177: mod_base = OTHER; note = Uracil
  modified_base 184: mod_base = OTHER; note = Uracil
  modified_base 187: mod_base = OTHER; note = Uracil
  modified_base 189: mod_base = OTHER; note = Uracil
  modified_base 190: mod_base = OTHER; note = Uracil
  modified_base 191: mod_base = OTHER; note = Uracil
  modified_base 192: mod_base = OTHER; note = Uracil
  modified_base 193: mod_base = OTHER; note = Uracil
  modified_base 195: mod_base = OTHER; note = Uracil
  modified_base 196: mod_base = OTHER; note = Uracil
  modified_base 200: mod_base = OTHER; note = Uracil
  modified_base 202: mod_base = OTHER; note = Uracil
  modified_base 204: mod_base = OTHER; note = Uracil
  modified_base 207: mod_base = OTHER; note = Uracil
  modified_base 209: mod_base = OTHER; note = Uracil
  modified_base 211: mod_base = OTHER; note = Uracil
  modified_base 216: mod_base = OTHER; note = Uracil
  modified_base 217: mod_base = OTHER; note = Uracil
  modified_base 220: mod_base = OTHER; note = Uracil
  modified_base 222: mod_base = OTHER; note = Uracil
  modified_base 228: mod_base = OTHER; note = Uracil
  modified_base 240: mod_base = OTHER; note = Uracil
  modified_base 247: mod_base = OTHER; note = Uracil
  modified_base 250: mod_base = OTHER; note = Uracil
  modified_base 252: mod_base = OTHER; note = Uracil
  modified_base 260: mod_base = OTHER; note = Uracil
  modified_base 264: mod_base = OTHER; note = Uracil
  modified_base 267: mod_base = OTHER; note = Uracil
  modified_base 268: mod_base = OTHER; note = Uracil
  modified_base 271: mod_base = OTHER; note = Uracil
  modified_base 274: mod_base = OTHER; note = Uracil
  modified_base 285: mod_base = OTHER; note = Uracil
  modified_base 286: mod_base = OTHER; note = Uracil
  modified_base 287: mod_base = OTHER; note = Uracil
  modified_base 289: mod_base = OTHER; note = Uracil
  modified_base 293: mod_base = OTHER; note = Uracil
  modified_base 296: mod_base = OTHER; note = Uracil
  modified_base 297: mod_base = OTHER; note = Uracil
  modified_base 299: mod_base = OTHER; note = Uracil
  modified_base 301: mod_base = OTHER; note = Uracil
  modified_base 302: mod_base = OTHER; note = Uracil
  modified_base 303: mod_base = OTHER; note = Uracil
  modified_base 314: mod_base = OTHER; note = Uracil
  modified_base 316: mod_base = OTHER; note = Uracil
  modified_base 319: mod_base = OTHER; note = Uracil
  modified_base 326: mod_base = OTHER; note = Uracil
  modified_base 332: mod_base = OTHER; note = Uracil
  modified_base 340: mod_base = OTHER; note = Uracil
  modified_base 342: mod_base = OTHER; note = Uracil
  modified_base 343: mod_base = OTHER; note = Uracil
  modified_base 347: mod_base = OTHER; note = Uracil
  modified_base 348: mod_base = OTHER; note = Uracil
  modified_base 354: mod_base = OTHER; note = Uracil
  modified_base 371: mod_base = OTHER; note = Uracil
  modified_base 373: mod_base = OTHER; note = Uracil
  modified_base 384: mod_base = OTHER; note = Uracil
  modified_base 387: mod_base = OTHER; note = Uracil
  modified_base 395: mod_base = OTHER; note = Uracil
  modified_base 397: mod_base = OTHER; note = Uracil
  modified_base 398: mod_base = OTHER; note = Uracil
  modified_base 399: mod_base = OTHER; note = Uracil
  modified_base 400: mod_base = OTHER; note = Uracil
  modified_base 409: mod_base = OTHER; note = Uracil
  modified_base 410: mod_base = OTHER; note = Uracil
  modified_base 411: mod_base = OTHER; note = Uracil
  modified_base 413: mod_base = OTHER; note = Uracil
  modified_base 423: mod_base = OTHER; note = Uracil
  modified_base 431: mod_base = OTHER; note = Uracil
  modified_base 432: mod_base = OTHER; note = Uracil
  modified_base 433: mod_base = OTHER; note = Uracil
  modified_base 440: mod_base = OTHER; note = Uracil
  modified_base 444: mod_base = OTHER; note = Uracil
  modified_base 446: mod_base = OTHER; note = Uracil
  modified_base 447: mod_base = OTHER; note = Uracil
  modified_base 451: mod_base = OTHER; note = Uracil
  modified_base 456: mod_base = OTHER; note = Uracil
  modified_base 457: mod_base = OTHER; note = Uracil
  modified_base 461: mod_base = OTHER; note = Uracil
  modified_base 463: mod_base = OTHER; note = Uracil
  modified_base 464: mod_base = OTHER; note = Uracil
  modified_base 465: mod_base = OTHER; note = Uracil
  modified_base 473: mod_base = OTHER; note = Uracil
  modified_base 474: mod_base = OTHER; note = Uracil
  modified_base 475: mod_base = OTHER; note = Uracil
  modified_base 477: mod_base = OTHER; note = Uracil
  modified_base 479: mod_base = OTHER; note = Uracil
  modified_base 485: mod_base = OTHER; note = Uracil
  modified_base 490: mod_base = OTHER; note = Uracil
  modified_base 498: mod_base = OTHER; note = Uracil
  modified_base 499: mod_base = OTHER; note = Uracil
  modified_base 508: mod_base = OTHER; note = Uracil
  modified_base 510: mod_base = OTHER; note = Uracil
  modified_base 512: mod_base = OTHER; note = Uracil
  modified_base 513: mod_base = OTHER; note = Uracil
  modified_base 514: mod_base = OTHER; note = Uracil
  modified_base 515: mod_base = OTHER; note = Uracil
  modified_base 517: mod_base = OTHER; note = Uracil
  modified_base 521: mod_base = OTHER; note = Uracil
  modified_base 523: mod_base = OTHER; note = Uracil
  modified_base 531: mod_base = OTHER; note = Uracil
  modified_base 532: mod_base = OTHER; note = Uracil
  modified_base 535: mod_base = OTHER; note = Uracil
  modified_base 537: mod_base = OTHER; note = Uracil
  modified_base 542: mod_base = OTHER; note = Uracil
  modified_base 544: mod_base = OTHER; note = Uracil
  modified_base 552: mod_base = OTHER; note = Uracil
  modified_base 553: mod_base = OTHER; note = Uracil
  modified_base 555: mod_base = OTHER; note = Uracil
  modified_base 561: mod_base = OTHER; note = Uracil
  modified_base 565: mod_base = OTHER; note = Uracil
  modified_base 568: mod_base = OTHER; note = Uracil
  modified_base 570: mod_base = OTHER; note = Uracil
  modified_base 571: mod_base = OTHER; note = Uracil
  modified_base 579: mod_base = OTHER; note = Uracil
  modified_base 580: mod_base = OTHER; note = Uracil
  modified_base 582: mod_base = OTHER; note = Uracil
  modified_base 588: mod_base = OTHER; note = Uracil
  modified_base 589: mod_base = OTHER; note = Uracil
  modified_base 591: mod_base = OTHER; note = Uracil
  modified_base 594: mod_base = OTHER; note = Uracil
  modified_base 597: mod_base = OTHER; note = Uracil
  modified_base 598: mod_base = OTHER; note = Uracil
  modified_base 601: mod_base = OTHER; note = Uracil
  modified_base 608: mod_base = OTHER; note = Uracil
  modified_base 609: mod_base = OTHER; note = Uracil
  modified_base 610: mod_base = OTHER; note = Uracil
  modified_base 617: mod_base = OTHER; note = Uracil
  modified_base 622: mod_base = OTHER; note = Uracil
  modified_base 624: mod_base = OTHER; note = Uracil
  modified_base 625: mod_base = OTHER; note = Uracil
  modified_base 627: mod_base = OTHER; note = Uracil
  modified_base 629: mod_base = OTHER; note = Uracil
  modified_base 631: mod_base = OTHER; note = Uracil
  modified_base 636: mod_base = OTHER; note = Uracil
  modified_base 637: mod_base = OTHER; note = Uracil
  modified_base 640: mod_base = OTHER; note = Uracil
  modified_base 642: mod_base = OTHER; note = Uracil
  modified_base 647: mod_base = OTHER; note = Uracil
  modified_base 649: mod_base = OTHER; note = Uracil
  modified_base 650: mod_base = OTHER; note = Uracil
  modified_base 657: mod_base = OTHER; note = Uracil
  modified_base 658: mod_base = OTHER; note = Uracil
  modified_base 659: mod_base = OTHER; note = Uracil
  modified_base 661: mod_base = OTHER; note = Uracil
  modified_base 664: mod_base = OTHER; note = Uracil
  modified_base 673: mod_base = OTHER; note = Uracil
  modified_base 674: mod_base = OTHER; note = Uracil
  modified_base 675: mod_base = OTHER; note = Uracil
  modified_base 678: mod_base = OTHER; note = Uracil
  modified_base 679: mod_base = OTHER; note = Uracil
  modified_base 680: mod_base = OTHER; note = Uracil
  modified_base 682: mod_base = OTHER; note = Uracil
  modified_base 683: mod_base = OTHER; note = Uracil
  modified_base 684: mod_base = OTHER; note = Uracil
  modified_base 685: mod_base = OTHER; note = Uracil
  modified_base 686: mod_base = OTHER; note = Uracil
  modified_base 693: mod_base = OTHER; note = Uracil
  modified_base 694: mod_base = OTHER; note = Uracil
  modified_base 700: mod_base = OTHER; note = Uracil
  modified_base 702: mod_base = OTHER; note = Uracil
  modified_base 706: mod_base = OTHER; note = Uracil
  modified_base 709: mod_base = OTHER; note = Uracil
  modified_base 710: mod_base = OTHER; note = Uracil
  modified_base 711: mod_base = OTHER; note = Uracil
  modified_base 715: mod_base = OTHER; note = Uracil
  modified_base 716: mod_base = OTHER; note = Uracil
  modified_base 719: mod_base = OTHER; note = Uracil
  modified_base 720: mod_base = OTHER; note = Uracil
  modified_base 721: mod_base = OTHER; note = Uracil
  modified_base 723: mod_base = OTHER; note = Uracil
  modified_base 727: mod_base = OTHER; note = Uracil
  modified_base 730: mod_base = OTHER; note = Uracil
  modified_base 738: mod_base = OTHER; note = Uracil
  modified_base 741: mod_base = OTHER; note = Uracil
  modified_base 742: mod_base = OTHER; note = Uracil
  modified_base 743: mod_base = OTHER; note = Uracil
  modified_base 744: mod_base = OTHER; note = Uracil
  modified_base 749: mod_base = OTHER; note = Uracil
  modified_base 751: mod_base = OTHER; note = Uracil
  modified_base 752: mod_base = OTHER; note = Uracil
  modified_base 756: mod_base = OTHER; note = Uracil
  modified_base 758: mod_base = OTHER; note = Uracil
  modified_base 768: mod_base = OTHER; note = Uracil
  modified_base 770: mod_base = OTHER; note = Uracil
  modified_base 771: mod_base = OTHER; note = Uracil
  modified_base 772: mod_base = OTHER; note = Uracil
  modified_base 774: mod_base = OTHER; note = Uracil
  modified_base 776: mod_base = OTHER; note = Uracil
  modified_base 787: mod_base = OTHER; note = Uracil
  modified_base 794: mod_base = OTHER; note = Uracil
  modified_base 795: mod_base = OTHER; note = Uracil
  modified_base 796: mod_base = OTHER; note = Uracil
  modified_base 803: mod_base = OTHER; note = Uracil
  modified_base 804: mod_base = OTHER; note = Uracil
  modified_base 805: mod_base = OTHER; note = Uracil
  modified_base 809: mod_base = OTHER; note = Uracil
  modified_base 811: mod_base = OTHER; note = Uracil
  modified_base 831: mod_base = OTHER; note = Uracil
  modified_base 833: mod_base = OTHER; note = Uracil
  modified_base 834: mod_base = OTHER; note = Uracil
  modified_base 838: mod_base = OTHER; note = Uracil
  modified_base 841: mod_base = OTHER; note = Uracil
  modified_base 842: mod_base = OTHER; note = Uracil
  modified_base 847: mod_base = OTHER; note = Uracil
  modified_base 872: mod_base = OTHER; note = Uracil
  modified_base 876: mod_base = OTHER; note = Uracil
  modified_base 885: mod_base = OTHER; note = Uracil
  modified_base 887: mod_base = OTHER; note = Uracil
  modified_base 889: mod_base = OTHER; note = Uracil
  modified_base 895: mod_base = OTHER; note = Uracil
  modified_base 897: mod_base = OTHER; note = Uracil
  modified_base 914: mod_base = OTHER; note = Uracil
  modified_base 915: mod_base = OTHER; note = Uracil
  modified_base 916: mod_base = OTHER; note = Uracil
  modified_base 919: mod_base = OTHER; note = Uracil
  modified_base 929: mod_base = OTHER; note = Uracil
  modified_base 934: mod_base = OTHER; note = Uracil
  modified_base 937: mod_base = OTHER; note = Uracil
  modified_base 941: mod_base = OTHER; note = Uracil
  modified_base 945: mod_base = OTHER; note = Uracil
  modified_base 953: mod_base = OTHER; note = Uracil
  modified_base 956: mod_base = OTHER; note = Uracil
  modified_base 958: mod_base = OTHER; note = Uracil
  modified_base 962: mod_base = OTHER; note = Uracil
  modified_base 964: mod_base = OTHER; note = Uracil
  modified_base 965: mod_base = OTHER; note = Uracil
  modified_base 966: mod_base = OTHER; note = Uracil
  modified_base 979: mod_base = OTHER; note = Uracil
  modified_base 980: mod_base = OTHER; note = Uracil
  modified_base 986: mod_base = OTHER; note = Uracil
  modified_base 991: mod_base = OTHER; note = Uracil
  modified_base 992: mod_base = OTHER; note = Uracil
  modified_base 997: mod_base = OTHER; note = Uracil
  modified_base 1001: mod_base = OTHER; note = Uracil
  modified_base 1005: mod_base = OTHER; note = Uracil
  modified_base 1006: mod_base = OTHER; note = Uracil
  modified_base 1015: mod_base = OTHER; note = Uracil
  modified_base 1016: mod_base = OTHER; note = Uracil
  modified_base 1023: mod_base = OTHER; note = Uracil
  modified_base 1034: mod_base = OTHER; note = Uracil
  modified_base 1037: mod_base = OTHER; note = Uracil
  modified_base 1039: mod_base = OTHER; note = Uracil
  modified_base 1040: mod_base = OTHER; note = Uracil
  modified_base 1045: mod_base = OTHER; note = Uracil
  modified_base 1048: mod_base = OTHER; note = Uracil
  modified_base 1065: mod_base = OTHER; note = Uracil
  modified_base 1075: mod_base = OTHER; note = Uracil
  modified_base 1077: mod_base = OTHER; note = Uracil
  modified_base 1078: mod_base = OTHER; note = Uracil
  modified_base 1083: mod_base = OTHER; note = Uracil
  modified_base 1099: mod_base = OTHER; note = Uracil
  modified_base 1102: mod_base = OTHER; note = Uracil
  modified_base 1103: mod_base = OTHER; note = Uracil
  modified_base 1117: mod_base = OTHER; note = Uracil
  modified_base 1125: mod_base = OTHER; note = Uracil
  modified_base 1134: mod_base = OTHER; note = Uracil
  modified_base 1138: mod_base = OTHER; note = Uracil
  modified_base 1140: mod_base = OTHER; note = Uracil
  modified_base 1143: mod_base = OTHER; note = Uracil
  modified_base 1145: mod_base = OTHER; note = Uracil
  modified_base 1146: mod_base = OTHER; note = Uracil
  modified_base 1147: mod_base = OTHER; note = Uracil
  modified_base 1148: mod_base = OTHER; note = Uracil
  modified_base 1157: mod_base = OTHER; note = Uracil
  modified_base 1161: mod_base = OTHER; note = Uracil
  modified_base 1165: mod_base = OTHER; note = Uracil
  modified_base 1166: mod_base = OTHER; note = Uracil
  modified_base 1172: mod_base = OTHER; note = Uracil
  modified_base 1173: mod_base = OTHER; note = Uracil
  modified_base 1183: mod_base = OTHER; note = Uracil
  modified_base 1184: mod_base = OTHER; note = Uracil
  modified_base 1186: mod_base = OTHER; note = Uracil
  modified_base 1191: mod_base = OTHER; note = Uracil
  modified_base 1199: mod_base = OTHER; note = Uracil
  modified_base 1205: mod_base = OTHER; note = Uracil
  modified_base 1207: mod_base = OTHER; note = Uracil
  modified_base 1208: mod_base = OTHER; note = Uracil
  modified_base 1210: mod_base = OTHER; note = Uracil
  modified_base 1213: mod_base = OTHER; note = Uracil
  modified_base 1216: mod_base = OTHER; note = Uracil
  modified_base 1239: mod_base = OTHER; note = Uracil
  modified_base 1251: mod_base = OTHER; note = Uracil
  modified_base 1254: mod_base = OTHER; note = Uracil
  modified_base 1255: mod_base = OTHER; note = Uracil
  modified_base 1256: mod_base = OTHER; note = Uracil
  modified_base 1259: mod_base = OTHER; note = Uracil
  modified_base 1270: mod_base = OTHER; note = Uracil
  modified_base 1272: mod_base = OTHER; note = Uracil
  modified_base 1273: mod_base = OTHER; note = Uracil
  modified_base 1279: mod_base = OTHER; note = Uracil
  modified_base 1280: mod_base = OTHER; note = Uracil
  modified_base 1283: mod_base = OTHER; note = Uracil
  modified_base 1284: mod_base = OTHER; note = Uracil
  modified_base 1289: mod_base = OTHER; note = Uracil
  modified_base 1295: mod_base = OTHER; note = Uracil
  modified_base 1307: mod_base = OTHER; note = Uracil
  modified_base 1308: mod_base = OTHER; note = Uracil
  modified_base 1314: mod_base = OTHER; note = Uracil
  modified_base 1315: mod_base = OTHER; note = Uracil
  modified_base 1318: mod_base = OTHER; note = Uracil
  modified_base 1327: mod_base = OTHER; note = Uracil
  modified_base 1336: mod_base = OTHER; note = Uracil
  modified_base 1345: mod_base = OTHER; note = Uracil
  modified_base 1352: mod_base = OTHER; note = Uracil
  modified_base 1358: mod_base = OTHER; note = Uracil
  modified_base 1359: mod_base = OTHER; note = Uracil
  modified_base 1361: mod_base = OTHER; note = Uracil
  modified_base 1363: mod_base = OTHER; note = Uracil
  modified_base 1365: mod_base = OTHER; note = Uracil
  modified_base 1366: mod_base = OTHER; note = Uracil
  modified_base 1367: mod_base = OTHER; note = Uracil
  modified_base 1393: mod_base = OTHER; note = Uracil
  modified_base 1394: mod_base = OTHER; note = Uracil
  modified_base 1395: mod_base = OTHER; note = Uracil
  modified_base 1396: mod_base = OTHER; note = Uracil
  modified_base 1409: mod_base = OTHER; note = Uracil
  modified_base 1411: mod_base = OTHER; note = Uracil
  modified_base 1420: mod_base = OTHER; note = Uracil
  modified_base 1429: mod_base = OTHER; note = Uracil
  modified_base 1431: mod_base = OTHER; note = Uracil
  modified_base 1433: mod_base = OTHER; note = Uracil
  modified_base 1444: mod_base = OTHER; note = Uracil
  modified_base 1452: mod_base = OTHER; note = Uracil
  modified_base 1457: mod_base = OTHER; note = Uracil
  modified_base 1469: mod_base = OTHER; note = Uracil
  modified_base 1470: mod_base = OTHER; note = Uracil
  modified_base 1471: mod_base = OTHER; note = Uracil
  modified_base 1476: mod_base = OTHER; note = Uracil
  modified_base 1484: mod_base = OTHER; note = Uracil
  modified_base 1493: mod_base = OTHER; note = Uracil
  modified_base 1494: mod_base = OTHER; note = Uracil
  modified_base 1502: mod_base = OTHER; note = Uracil
  modified_base 1504: mod_base = OTHER; note = Uracil
  modified_base 1514: mod_base = OTHER; note = Uracil
  modified_base 1522: mod_base = OTHER; note = Uracil
  modified_base 1524: mod_base = OTHER; note = Uracil
  modified_base 1525: mod_base = OTHER; note = Uracil
  modified_base 1527: mod_base = OTHER; note = Uracil
  modified_base 1528: mod_base = OTHER; note = Uracil
  modified_base 1544: mod_base = OTHER; note = Uracil
  modified_base 1546: mod_base = OTHER; note = Uracil
  modified_base 1548: mod_base = OTHER; note = Uracil
  modified_base 1561: mod_base = OTHER; note = Uracil
  modified_base 1599: mod_base = OTHER; note = Uracil
  modified_base 1621: mod_base = OTHER; note = Uracil
  modified_base 1625: mod_base = OTHER; note = Uracil
  modified_base 1636: mod_base = OTHER; note = Uracil
  modified_base 1639: mod_base = OTHER; note = Uracil
  modified_base 1648: mod_base = OTHER; note = Uracil
  modified_base 1651: mod_base = OTHER; note = Uracil
  modified_base 1653: mod_base = OTHER; note = Uracil
  modified_base 1656: mod_base = OTHER; note = Uracil
  modified_base 1658: mod_base = OTHER; note = Uracil
  modified_base 1660: mod_base = OTHER; note = Uracil
  modified_base 1663: mod_base = OTHER; note = Uracil
  modified_base 1672: mod_base = OTHER; note = Uracil
  modified_base 1676: mod_base = OTHER; note = Uracil
  modified_base 1678: mod_base = OTHER; note = Uracil
  modified_base 1687: mod_base = OTHER; note = Uracil
  modified_base 1699: mod_base = OTHER; note = Uracil
  modified_base 1702: mod_base = OTHER; note = Uracil
  modified_base 1711: mod_base = OTHER; note = Uracil
  modified_base 1713: mod_base = OTHER; note = Uracil
  modified_base 1725: mod_base = OTHER; note = Uracil
  modified_base 1726: mod_base = OTHER; note = Uracil
  modified_base 1733: mod_base = OTHER; note = Uracil
  modified_base 1740: mod_base = OTHER; note = Uracil
  modified_base 1778: mod_base = OTHER; note = Uracil
  modified_base 1779: mod_base = OTHER; note = Uracil
  modified_base 1787: mod_base = OTHER; note = Uracil
  modified_base 1790: mod_base = OTHER; note = Uracil
  modified_base 1791: mod_base = OTHER; note = Uracil
  modified_base 1793: mod_base = OTHER; note = Uracil
  modified_base 1796: mod_base = OTHER; note = Uracil
  modified_base 1800: mod_base = OTHER; note = Uracil
  modified_base 1802: mod_base = OTHER; note = Uracil
  modified_base 1808: mod_base = OTHER; note = Uracil
  modified_base 1815: mod_base = OTHER; note = Uracil
  modified_base 1823: mod_base = OTHER; note = Uracil
  modified_base 1831: mod_base = OTHER; note = Uracil
  modified_base 1836: mod_base = OTHER; note = Uracil
  modified_base 1844: mod_base = OTHER; note = Uracil
  modified_base 1845: mod_base = OTHER; note = Uracil
  modified_base 1849: mod_base = OTHER; note = Uracil
  modified_base 1853: mod_base = OTHER; note = Uracil
  modified_base 1863: mod_base = OTHER; note = Uracil
  modified_base 1871: mod_base = OTHER; note = Uracil
  modified_base 1874: mod_base = OTHER; note = Uracil
  modified_base 1898: mod_base = OTHER; note = Uracil
  modified_base 1904: mod_base = OTHER; note = Uracil
  modified_base 1917: mod_base = OTHER; note = Uracil
  modified_base 1920: mod_base = OTHER; note = Uracil
  modified_base 1930: mod_base = OTHER; note = Uracil
  modified_base 1937: mod_base = OTHER; note = Uracil
  modified_base 1938: mod_base = OTHER; note = Uracil
  modified_base 1942: mod_base = OTHER; note = Uracil
  modified_base 1958: mod_base = OTHER; note = Uracil
  modified_base 1959: mod_base = OTHER; note = Uracil
  modified_base 1964: mod_base = OTHER; note = Uracil
  modified_base 1973: mod_base = OTHER; note = Uracil
  modified_base 1974: mod_base = OTHER; note = Uracil
  modified_base 1992: mod_base = OTHER; note = Uracil
  modified_base 2001: mod_base = OTHER; note = Uracil
  modified_base 2007: mod_base = OTHER; note = Uracil
  modified_base 2014: mod_base = OTHER; note = Uracil
  modified_base 2015: mod_base = OTHER; note = Uracil
  modified_base 2021: mod_base = OTHER; note = Uracil
  modified_base 2025: mod_base = OTHER; note = Uracil
  modified_base 2039: mod_base = OTHER; note = Uracil
  modified_base 2049: mod_base = OTHER; note = Uracil
  modified_base 2054: mod_base = OTHER; note = Uracil
  modified_base 2056: mod_base = OTHER; note = Uracil
  modified_base 2062: mod_base = OTHER; note = Uracil
  modified_base 2074: mod_base = OTHER; note = Uracil
  modified_base 2076: mod_base = OTHER; note = Uracil
  modified_base 2079: mod_base = OTHER; note = Uracil
  modified_base 2080: mod_base = OTHER; note = Uracil
  modified_base 2095: mod_base = OTHER; note = Uracil
  modified_base 2097: mod_base = OTHER; note = Uracil
  modified_base 2098: mod_base = OTHER; note = Uracil
  modified_base 2099: mod_base = OTHER; note = Uracil
  modified_base 2104: mod_base = OTHER; note = Uracil
  modified_base 2107: mod_base = OTHER; note = Uracil
  modified_base 2108: mod_base = OTHER; note = Uracil
  modified_base 2119: mod_base = OTHER; note = Uracil
  modified_base 2125: mod_base = OTHER; note = Uracil
  modified_base 2148: mod_base = OTHER; note = Uracil
  modified_base 2153: mod_base = OTHER; note = Uracil
  modified_base 2162: mod_base = OTHER; note = Uracil
  modified_base 2163: mod_base = OTHER; note = Uracil
  modified_base 2166: mod_base = OTHER; note = Uracil
  modified_base 2169: mod_base = OTHER; note = Uracil
  modified_base 2170: mod_base = OTHER; note = Uracil
  modified_base 2173: mod_base = OTHER; note = Uracil
  modified_base 2174: mod_base = OTHER; note = Uracil
  modified_base 2175: mod_base = OTHER; note = Uracil
  modified_base 2187: mod_base = OTHER; note = Uracil
  modified_base 2189: mod_base = OTHER; note = Uracil
  modified_base 2190: mod_base = OTHER; note = Uracil
  modified_base 2192: mod_base = OTHER; note = Uracil
  modified_base 2196: mod_base = OTHER; note = Uracil
  modified_base 2201: mod_base = OTHER; note = Uracil
  modified_base 2202: mod_base = OTHER; note = Uracil
  modified_base 2206: mod_base = OTHER; note = Uracil
  modified_base 2214: mod_base = OTHER; note = Uracil
  modified_base 2220: mod_base = OTHER; note = Uracil
  modified_base 2225: mod_base = OTHER; note = Uracil
  modified_base 2227: mod_base = OTHER; note = Uracil
  modified_base 2229: mod_base = OTHER; note = Uracil
  modified_base 2230: mod_base = OTHER; note = Uracil
  modified_base 2234: mod_base = OTHER; note = Uracil
  modified_base 2236: mod_base = OTHER; note = Uracil
  modified_base 2254: mod_base = OTHER; note = Uracil
  modified_base 2258: mod_base = OTHER; note = Uracil
  modified_base 2262: mod_base = OTHER; note = Uracil
  modified_base 2269: mod_base = OTHER; note = Uracil
  modified_base 2277: mod_base = OTHER; note = Uracil
  modified_base 2280: mod_base = OTHER; note = Uracil
  modified_base 2281: mod_base = OTHER; note = Uracil
  modified_base 2286: mod_base = OTHER; note = Uracil
  modified_base 2289: mod_base = OTHER; note = Uracil
  modified_base 2293: mod_base = OTHER; note = Uracil
  modified_base 2297: mod_base = OTHER; note = Uracil
  modified_base 2298: mod_base = OTHER; note = Uracil
  modified_base 2303: mod_base = OTHER; note = Uracil
  modified_base 2304: mod_base = OTHER; note = Uracil
  modified_base 2307: mod_base = OTHER; note = Uracil
  modified_base 2313: mod_base = OTHER; note = Uracil
  modified_base 2322: mod_base = OTHER; note = Uracil
  modified_base 2324: mod_base = OTHER; note = Uracil
  modified_base 2325: mod_base = OTHER; note = Uracil
  modified_base 2331: mod_base = OTHER; note = Uracil
  modified_base 2332: mod_base = OTHER; note = Uracil
  modified_base 2335: mod_base = OTHER; note = Uracil
  modified_base 2336: mod_base = OTHER; note = Uracil
  modified_base 2337: mod_base = OTHER; note = Uracil
  modified_base 2344: mod_base = OTHER; note = Uracil
  modified_base 2346: mod_base = OTHER; note = Uracil
  modified_base 2361: mod_base = OTHER; note = Uracil
  modified_base 2364: mod_base = OTHER; note = Uracil
  modified_base 2374: mod_base = OTHER; note = Uracil
  modified_base 2375: mod_base = OTHER; note = Uracil
  modified_base 2376: mod_base = OTHER; note = Uracil
  modified_base 2378: mod_base = OTHER; note = Uracil
  modified_base 2388: mod_base = OTHER; note = Uracil
  modified_base 2393: mod_base = OTHER; note = Uracil
  modified_base 2394: mod_base = OTHER; note = Uracil
  modified_base 2396: mod_base = OTHER; note = Uracil
  modified_base 2397: mod_base = OTHER; note = Uracil
  modified_base 2400: mod_base = OTHER; note = Uracil
  modified_base 2430: mod_base = OTHER; note = Uracil
  modified_base 2434: mod_base = OTHER; note = Uracil
  modified_base 2444: mod_base = OTHER; note = Uracil
  modified_base 2451: mod_base = OTHER; note = Uracil
  modified_base 2454: mod_base = OTHER; note = Uracil
  modified_base 2457: mod_base = OTHER; note = Uracil
  modified_base 2463: mod_base = OTHER; note = Uracil
  modified_base 2464: mod_base = OTHER; note = Uracil
  modified_base 2465: mod_base = OTHER; note = Uracil
  modified_base 2469: mod_base = OTHER; note = Uracil
  modified_base 2472: mod_base = OTHER; note = Uracil
  modified_base 2476: mod_base = OTHER; note = Uracil
  modified_base 2481: mod_base = OTHER; note = Uracil
  modified_base 2484: mod_base = OTHER; note = Uracil
  modified_base 2493: mod_base = OTHER; note = Uracil
  modified_base 2494: mod_base = OTHER; note = Uracil
  modified_base 2496: mod_base = OTHER; note = Uracil
  modified_base 2498: mod_base = OTHER; note = Uracil
  modified_base 2499: mod_base = OTHER; note = Uracil
  modified_base 2523: mod_base = OTHER; note = Uracil
  modified_base 2524: mod_base = OTHER; note = Uracil
  modified_base 2529: mod_base = OTHER; note = Uracil
  modified_base 2530: mod_base = OTHER; note = Uracil
  modified_base 2531: mod_base = OTHER; note = Uracil
  modified_base 2541: mod_base = OTHER; note = Uracil
  modified_base 2548: mod_base = OTHER; note = Uracil
  modified_base 2557: mod_base = OTHER; note = Uracil
  modified_base 2558: mod_base = OTHER; note = Uracil
  modified_base 2559: mod_base = OTHER; note = Uracil
  modified_base 2564: mod_base = OTHER; note = Uracil
  modified_base 2565: mod_base = OTHER; note = Uracil
  modified_base 2567: mod_base = OTHER; note = Uracil
  modified_base 2583: mod_base = OTHER; note = Uracil
  modified_base 2586: mod_base = OTHER; note = Uracil
  modified_base 2591: mod_base = OTHER; note = Uracil
  modified_base 2592: mod_base = OTHER; note = Uracil
  modified_base 2593: mod_base = OTHER; note = Uracil
  modified_base 2595: mod_base = OTHER; note = Uracil
  modified_base 2602: mod_base = OTHER; note = Uracil
  modified_base 2603: mod_base = OTHER; note = Uracil
  modified_base 2604: mod_base = OTHER; note = Uracil
  modified_base 2611: mod_base = OTHER; note = Uracil
  modified_base 2616: mod_base = OTHER; note = Uracil
  modified_base 2623: mod_base = OTHER; note = Uracil
  modified_base 2637: mod_base = OTHER; note = Uracil
  modified_base 2640: mod_base = OTHER; note = Uracil
  modified_base 2641: mod_base = OTHER; note = Uracil
  modified_base 2647: mod_base = OTHER; note = Uracil
  modified_base 2648: mod_base = OTHER; note = Uracil
  modified_base 2657: mod_base = OTHER; note = Uracil
  modified_base 2660: mod_base = OTHER; note = Uracil
  modified_base 2661: mod_base = OTHER; note = Uracil
  modified_base 2674: mod_base = OTHER; note = Uracil
  modified_base 2675: mod_base = OTHER; note = Uracil
  modified_base 2678: mod_base = OTHER; note = Uracil
  modified_base 2683: mod_base = OTHER; note = Uracil
  modified_base 2684: mod_base = OTHER; note = Uracil
  modified_base 2685: mod_base = OTHER; note = Uracil
  modified_base 2690: mod_base = OTHER; note = Uracil
  modified_base 2695: mod_base = OTHER; note = Uracil
  modified_base 2697: mod_base = OTHER; note = Uracil
  modified_base 2698: mod_base = OTHER; note = Uracil
  modified_base 2705: mod_base = OTHER; note = Uracil
  modified_base 2709: mod_base = OTHER; note = Uracil
  modified_base 2712: mod_base = OTHER; note = Uracil
  modified_base 2713: mod_base = OTHER; note = Uracil
  modified_base 2722: mod_base = OTHER; note = Uracil
  modified_base 2724: mod_base = OTHER; note = Uracil
  modified_base 2727: mod_base = OTHER; note = Uracil
  modified_base 2730: mod_base = OTHER; note = Uracil
  modified_base 2737: mod_base = OTHER; note = Uracil
  modified_base 2742: mod_base = OTHER; note = Uracil
  modified_base 2743: mod_base = OTHER; note = Uracil
  modified_base 2748: mod_base = OTHER; note = Uracil
  modified_base 2754: mod_base = OTHER; note = Uracil
  modified_base 2761: mod_base = OTHER; note = Uracil
  modified_base 2763: mod_base = OTHER; note = Uracil
  modified_base 2764: mod_base = OTHER; note = Uracil
  modified_base 2774: mod_base = OTHER; note = Uracil
  modified_base 2775: mod_base = OTHER; note = Uracil
  modified_base 2780: mod_base = OTHER; note = Uracil
  modified_base 2783: mod_base = OTHER; note = Uracil
  modified_base 2785: mod_base = OTHER; note = Uracil
  modified_base 2786: mod_base = OTHER; note = Uracil
  modified_base 2789: mod_base = OTHER; note = Uracil
  modified_base 2790: mod_base = OTHER; note = Uracil
  modified_base 2795: mod_base = OTHER; note = Uracil
  modified_base 2797: mod_base = OTHER; note = Uracil
  modified_base 2799: mod_base = OTHER; note = Uracil
  modified_base 2800: mod_base = OTHER; note = Uracil
  modified_base 2801: mod_base = OTHER; note = Uracil
  modified_base 2807: mod_base = OTHER; note = Uracil
  modified_base 2811: mod_base = OTHER; note = Uracil
  modified_base 2814: mod_base = OTHER; note = Uracil
  modified_base 2816: mod_base = OTHER; note = Uracil
  modified_base 2820: mod_base = OTHER; note = Uracil
  modified_base 2824: mod_base = OTHER; note = Uracil
  modified_base 2827: mod_base = OTHER; note = Uracil
  modified_base 2830: mod_base = OTHER; note = Uracil
  modified_base 2835: mod_base = OTHER; note = Uracil
  modified_base 2836: mod_base = OTHER; note = Uracil
  modified_base 2837: mod_base = OTHER; note = Uracil
  modified_base 2838: mod_base = OTHER; note = Uracil
  modified_base 2851: mod_base = OTHER; note = Uracil
  modified_base 2859: mod_base = OTHER; note = Uracil
  modified_base 2860: mod_base = OTHER; note = Uracil
  modified_base 2869: mod_base = OTHER; note = Uracil
  modified_base 2876: mod_base = OTHER; note = Uracil
  modified_base 2881: mod_base = OTHER; note = Uracil
  modified_base 2884: mod_base = OTHER; note = Uracil
  modified_base 2885: mod_base = OTHER; note = Uracil
  modified_base 2891: mod_base = OTHER; note = Uracil
  modified_base 2897: mod_base = OTHER; note = Uracil
  modified_base 2904: mod_base = OTHER; note = Uracil
  modified_base 2910: mod_base = OTHER; note = Uracil
  modified_base 2911: mod_base = OTHER; note = Uracil
  modified_base 2913: mod_base = OTHER; note = Uracil
  modified_base 2921: mod_base = OTHER; note = Uracil
  modified_base 2922: mod_base = OTHER; note = Uracil
  modified_base 2923: mod_base = OTHER; note = Uracil
  modified_base 2932: mod_base = OTHER; note = Uracil
  modified_base 2935: mod_base = OTHER; note = Uracil
  modified_base 2939: mod_base = OTHER; note = Uracil
  modified_base 2944: mod_base = OTHER; note = Uracil
  modified_base 2946: mod_base = OTHER; note = Uracil
  modified_base 2952: mod_base = OTHER; note = Uracil
  modified_base 2959: mod_base = OTHER; note = Uracil
  modified_base 2967: mod_base = OTHER; note = Uracil
  modified_base 2972: mod_base = OTHER; note = Uracil
  modified_base 2977: mod_base = OTHER; note = Uracil
  modified_base 2983: mod_base = OTHER; note = Uracil
  modified_base 2988: mod_base = OTHER; note = Uracil
  modified_base 2996: mod_base = OTHER; note = Uracil
  modified_base 3006: mod_base = OTHER; note = Uracil
  modified_base 3010: mod_base = OTHER; note = Uracil
  modified_base 3011: mod_base = OTHER; note = Uracil
  modified_base 3012: mod_base = OTHER; note = Uracil
  modified_base 3018: mod_base = OTHER; note = Uracil
  modified_base 3019: mod_base = OTHER; note = Uracil
  modified_base 3024: mod_base = OTHER; note = Uracil
  modified_base 3025: mod_base = OTHER; note = Uracil
  modified_base 3026: mod_base = OTHER; note = Uracil
  modified_base 3036: mod_base = OTHER; note = Uracil
  modified_base 3041: mod_base = OTHER; note = Uracil
  modified_base 3042: mod_base = OTHER; note = Uracil
  modified_base 3044: mod_base = OTHER; note = Uracil
  modified_base 3047: mod_base = OTHER; note = Uracil
  modified_base 3049: mod_base = OTHER; note = Uracil
  modified_base 3054: mod_base = OTHER; note = Uracil
  modified_base 3056: mod_base = OTHER; note = Uracil
  modified_base 3057: mod_base = OTHER; note = Uracil
  modified_base 3058: mod_base = OTHER; note = Uracil
  modified_base 3072: mod_base = OTHER; note = Uracil
  modified_base 3078: mod_base = OTHER; note = Uracil
  modified_base 3082: mod_base = OTHER; note = Uracil
  modified_base 3084: mod_base = OTHER; note = Uracil
  modified_base 3099: mod_base = OTHER; note = Uracil
  modified_base 3102: mod_base = OTHER; note = Uracil
  modified_base 3104: mod_base = OTHER; note = Uracil
  modified_base 3111: mod_base = OTHER; note = Uracil
  modified_base 3114: mod_base = OTHER; note = Uracil
  modified_base 3116: mod_base = OTHER; note = Uracil
  modified_base 3118: mod_base = OTHER; note = Uracil
  modified_base 3122: mod_base = OTHER; note = Uracil
  modified_base 3127: mod_base = OTHER; note = Uracil
  modified_base 3145: mod_base = OTHER; note = Uracil
  modified_base 3150: mod_base = OTHER; note = Uracil
  modified_base 3153: mod_base = OTHER; note = Uracil
  modified_base 3154: mod_base = OTHER; note = Uracil
  modified_base 3155: mod_base = OTHER; note = Uracil
  modified_base 3161: mod_base = OTHER; note = Uracil
  modified_base 3164: mod_base = OTHER; note = Uracil
  modified_base 3165: mod_base = OTHER; note = Uracil
  modified_base 3172: mod_base = OTHER; note = Uracil
  modified_base 3173: mod_base = OTHER; note = Uracil
  modified_base 3175: mod_base = OTHER; note = Uracil
  modified_base 3176: mod_base = OTHER; note = Uracil
  modified_base 3177: mod_base = OTHER; note = Uracil
  modified_base 3179: mod_base = OTHER; note = Uracil
  modified_base 3183: mod_base = OTHER; note = Uracil
  modified_base 3184: mod_base = OTHER; note = Uracil
  modified_base 3186: mod_base = OTHER; note = Uracil
  modified_base 3194: mod_base = OTHER; note = Uracil
  modified_base 3195: mod_base = OTHER; note = Uracil
  modified_base 3205: mod_base = OTHER; note = Uracil
  modified_base 3212: mod_base = OTHER; note = Uracil
  modified_base 3216: mod_base = OTHER; note = Uracil
  modified_base 3217: mod_base = OTHER; note = Uracil
  modified_base 3222: mod_base = OTHER; note = Uracil
  modified_base 3223: mod_base = OTHER; note = Uracil
  modified_base 3225: mod_base = OTHER; note = Uracil
  modified_base 3226: mod_base = OTHER; note = Uracil
  modified_base 3227: mod_base = OTHER; note = Uracil
  modified_base 3230: mod_base = OTHER; note = Uracil
  modified_base 3233: mod_base = OTHER; note = Uracil
  modified_base 3236: mod_base = OTHER; note = Uracil
  modified_base 3237: mod_base = OTHER; note = Uracil
  modified_base 3240: mod_base = OTHER; note = Uracil
  modified_base 3249: mod_base = OTHER; note = Uracil
  modified_base 3252: mod_base = OTHER; note = Uracil
  modified_base 3264: mod_base = OTHER; note = Uracil
  modified_base 3275: mod_base = OTHER; note = Uracil
  modified_base 3291: mod_base = OTHER; note = Uracil
  modified_base 3292: mod_base = OTHER; note = Uracil
  modified_base 3298: mod_base = OTHER; note = Uracil
  modified_base 3302: mod_base = OTHER; note = Uracil
  modified_base 3306: mod_base = OTHER; note = Uracil
  modified_base 3310: mod_base = OTHER; note = Uracil
  modified_base 3311: mod_base = OTHER; note = Uracil
  modified_base 3317: mod_base = OTHER; note = Uracil
  modified_base 3331: mod_base = OTHER; note = Uracil
  modified_base 3333: mod_base = OTHER; note = Uracil
  modified_base 3334: mod_base = OTHER; note = Uracil
  modified_base 3344: mod_base = OTHER; note = Uracil
  modified_base 3346: mod_base = OTHER; note = Uracil
  modified_base 3352: mod_base = OTHER; note = Uracil
  modified_base 3356: mod_base = OTHER; note = Uracil
  modified_base 3357: mod_base = OTHER; note = Uracil
  modified_base 3363: mod_base = OTHER; note = Uracil
  modified_base 3364: mod_base = OTHER; note = Uracil
  modified_base 3367: mod_base = OTHER; note = Uracil
  modified_base 3375: mod_base = OTHER; note = Uracil
  modified_base 3376: mod_base = OTHER; note = Uracil
  modified_base 3377: mod_base = OTHER; note = Uracil
  modified_base 3378: mod_base = OTHER; note = Uracil
  modified_base 3387: mod_base = OTHER; note = Uracil
  modified_base 3388: mod_base = OTHER; note = Uracil
  modified_base 3390: mod_base = OTHER; note = Uracil
  modified_base 3391: mod_base = OTHER; note = Uracil
  modified_base 3393: mod_base = OTHER; note = Uracil
  modified_base 3394: mod_base = OTHER; note = Uracil
  modified_base 3400: mod_base = OTHER; note = Uracil
  modified_base 3406: mod_base = OTHER; note = Uracil
  modified_base 3407: mod_base = OTHER; note = Uracil
  modified_base 3408: mod_base = OTHER; note = Uracil
  modified_base 3415: mod_base = OTHER; note = Uracil
  modified_base 3420: mod_base = OTHER; note = Uracil
  modified_base 3421: mod_base = OTHER; note = Uracil
  modified_base 3423: mod_base = OTHER; note = Uracil
  modified_base 3427: mod_base = OTHER; note = Uracil
  modified_base 3430: mod_base = OTHER; note = Uracil
  modified_base 3432: mod_base = OTHER; note = Uracil
  modified_base 3436: mod_base = OTHER; note = Uracil
  modified_base 3442: mod_base = OTHER; note = Uracil
  modified_base 3443: mod_base = OTHER; note = Uracil
  modified_base 3444: mod_base = OTHER; note = Uracil
  modified_base 3454: mod_base = OTHER; note = Uracil
  modified_base 3459: mod_base = OTHER; note = Uracil
  modified_base 3463: mod_base = OTHER; note = Uracil
  modified_base 3466: mod_base = OTHER; note = Uracil
  modified_base 3467: mod_base = OTHER; note = Uracil
  modified_base 3470: mod_base = OTHER; note = Uracil
  modified_base 3474: mod_base = OTHER; note = Uracil
  modified_base 3479: mod_base = OTHER; note = Uracil
  modified_base 3482: mod_base = OTHER; note = Uracil
  modified_base 3486: mod_base = OTHER; note = Uracil
  modified_base 3497: mod_base = OTHER; note = Uracil
  modified_base 3501: mod_base = OTHER; note = Uracil
  modified_base 3537: mod_base = OTHER; note = Uracil
  modified_base 3544: mod_base = OTHER; note = Uracil
  modified_base 3546: mod_base = OTHER; note = Uracil
  modified_base 3554: mod_base = OTHER; note = Uracil
  modified_base 3557: mod_base = OTHER; note = Uracil
  modified_base 3558: mod_base = OTHER; note = Uracil
  modified_base 3560: mod_base = OTHER; note = Uracil
  modified_base 3563: mod_base = OTHER; note = Uracil
  modified_base 3571: mod_base = OTHER; note = Uracil
  modified_base 3573: mod_base = OTHER; note = Uracil
  modified_base 3615: mod_base = OTHER; note = Uracil
  modified_base 3633: mod_base = OTHER; note = Uracil
  modified_base 3635: mod_base = OTHER; note = Uracil
  modified_base 3636: mod_base = OTHER; note = Uracil
  modified_base 3639: mod_base = OTHER; note = Uracil
  modified_base 3654: mod_base = OTHER; note = Uracil
  modified_base 3655: mod_base = OTHER; note = Uracil
  modified_base 3667: mod_base = OTHER; note = Uracil
  modified_base 3668: mod_base = OTHER; note = Uracil
  modified_base 3669: mod_base = OTHER; note = Uracil
  modified_base 3671: mod_base = OTHER; note = Uracil
  modified_base 3672: mod_base = OTHER; note = Uracil
  modified_base 3673: mod_base = OTHER; note = Uracil
  modified_base 3674: mod_base = OTHER; note = Uracil
  modified_base 3676: mod_base = OTHER; note = Uracil
  modified_base 3687: mod_base = OTHER; note = Uracil
  modified_base 3689: mod_base = OTHER; note = Uracil
  modified_base 3699: mod_base = OTHER; note = Uracil
  modified_base 3700: mod_base = OTHER; note = Uracil
  modified_base 3713: mod_base = OTHER; note = Uracil
  modified_base 3718: mod_base = OTHER; note = Uracil
  modified_base 3719: mod_base = OTHER; note = Uracil
  modified_base 3729: mod_base = OTHER; note = Uracil
  modified_base 3731: mod_base = OTHER; note = Uracil
  modified_base 3733: mod_base = OTHER; note = Uracil
  modified_base 3739: mod_base = OTHER; note = Uracil
  modified_base 3748: mod_base = OTHER; note = Uracil
  modified_base 3749: mod_base = OTHER; note = Uracil
  modified_base 3753: mod_base = OTHER; note = Uracil
  modified_base 3756: mod_base = OTHER; note = Uracil
  modified_base 3761: mod_base = OTHER; note = Uracil
  modified_base 3764: mod_base = OTHER; note = Uracil
  modified_base 3772: mod_base = OTHER; note = Uracil
  modified_base 3777: mod_base = OTHER; note = Uracil
  modified_base 3781: mod_base = OTHER; note = Uracil
  modified_base 3783: mod_base = OTHER; note = Uracil
  modified_base 3788: mod_base = OTHER; note = Uracil
  modified_base 3790: mod_base = OTHER; note = Uracil
  modified_base 3794: mod_base = OTHER; note = Uracil
  modified_base 3797: mod_base = OTHER; note = Uracil
  modified_base 3800: mod_base = OTHER; note = Uracil
  modified_base 3801: mod_base = OTHER; note = Uracil
  modified_base 3802: mod_base = OTHER; note = Uracil
  modified_base 3803: mod_base = OTHER; note = Uracil
  modified_base 3808: mod_base = OTHER; note = Uracil
  modified_base 3809: mod_base = OTHER; note = Uracil
  modified_base 3812: mod_base = OTHER; note = Uracil
  modified_base 3814: mod_base = OTHER; note = Uracil
  modified_base 3823: mod_base = OTHER; note = Uracil
  modified_base 3825: mod_base = OTHER; note = Uracil
  modified_base 3826: mod_base = OTHER; note = Uracil
  modified_base 3834: mod_base = OTHER; note = Uracil
  modified_base 3835: mod_base = OTHER; note = Uracil
  modified_base 3840: mod_base = OTHER; note = Uracil
  modified_base 3841: mod_base = OTHER; note = Uracil
  modified_base 3845: mod_base = OTHER; note = Uracil
  modified_base 3849: mod_base = OTHER; note = Uracil
  modified_base 3850: mod_base = OTHER; note = Uracil
  modified_base 3857: mod_base = OTHER; note = Uracil
  modified_base 3868: mod_base = OTHER; note = Uracil
  modified_base 3870: mod_base = OTHER; note = Uracil
  modified_base 3875: mod_base = OTHER; note = Uracil
  modified_base 3880: mod_base = OTHER; note = Uracil
  modified_base 3884: mod_base = OTHER; note = Uracil
  modified_base 3885: mod_base = OTHER; note = Uracil
  modified_base 3886: mod_base = OTHER; note = Uracil
  modified_base 3889: mod_base = OTHER; note = Uracil
  modified_base 3894: mod_base = OTHER; note = Uracil
  modified_base 3896: mod_base = OTHER; note = Uracil
  modified_base 3904: mod_base = OTHER; note = Uracil
  modified_base 3905: mod_base = OTHER; note = Uracil
  modified_base 3911: mod_base = OTHER; note = Uracil
  modified_base 3934: mod_base = OTHER; note = Uracil
  modified_base 3935: mod_base = OTHER; note = Uracil
  modified_base 3936: mod_base = OTHER; note = Uracil
  modified_base 3943: mod_base = OTHER; note = Uracil
  modified_base 3945: mod_base = OTHER; note = Uracil
  modified_base 3946: mod_base = OTHER; note = Uracil
  modified_base 3950: mod_base = OTHER; note = Uracil
  modified_base 3964: mod_base = OTHER; note = Uracil
  modified_base 3974: mod_base = OTHER; note = Uracil
  modified_base 3975: mod_base = OTHER; note = Uracil
  modified_base 3988: mod_base = OTHER; note = Uracil
  modified_base 3989: mod_base = OTHER; note = Uracil
  modified_base 3995: mod_base = OTHER; note = Uracil
  modified_base 3998: mod_base = OTHER; note = Uracil
  modified_base 4000: mod_base = OTHER; note = Uracil
  modified_base 4011: mod_base = OTHER; note = Uracil
  modified_base 4014: mod_base = OTHER; note = Uracil
  modified_base 4015: mod_base = OTHER; note = Uracil
  modified_base 4017: mod_base = OTHER; note = Uracil
  modified_base 4018: mod_base = OTHER; note = Uracil
  modified_base 4021: mod_base = OTHER; note = Uracil
  modified_base 4025: mod_base = OTHER; note = Uracil
  modified_base 4029: mod_base = OTHER; note = Uracil
  modified_base 4032: mod_base = OTHER; note = Uracil
  modified_base 4033: mod_base = OTHER; note = Uracil
  modified_base 4035: mod_base = OTHER; note = Uracil
  modified_base 4036: mod_base = OTHER; note = Uracil
  modified_base 4037: mod_base = OTHER; note = Uracil
  modified_base 4043: mod_base = OTHER; note = Uracil
  modified_base 4049: mod_base = OTHER; note = Uracil
  modified_base 4054: mod_base = OTHER; note = Uracil
  modified_base 4060: mod_base = OTHER; note = Uracil
  modified_base 4061: mod_base = OTHER; note = Uracil
  modified_base 4068: mod_base = OTHER; note = Uracil
  modified_base 4075: mod_base = OTHER; note = Uracil
  modified_base 4079: mod_base = OTHER; note = Uracil
  modified_base 4110: mod_base = OTHER; note = Uracil
  modified_base 4111: mod_base = OTHER; note = Uracil
  modified_base 4113: mod_base = OTHER; note = Uracil
  modified_base 4117: mod_base = OTHER; note = Uracil
  modified_base 4119: mod_base = OTHER; note = Uracil
  modified_base 4144: mod_base = OTHER; note = Uracil
  modified_base 4146: mod_base = OTHER; note = Uracil
  modified_base 4149: mod_base = OTHER; note = Uracil
  modified_base 4150: mod_base = OTHER; note = Uracil
  modified_base 4151: mod_base = OTHER; note = Uracil
  modified_base 4152: mod_base = OTHER; note = Uracil
  modified_base 4154: mod_base = OTHER; note = Uracil
  modified_base 4157: mod_base = OTHER; note = Uracil
  modified_base 4159: mod_base = OTHER; note = Uracil
  modified_base 4161: mod_base = OTHER; note = Uracil
  modified_base 4164: mod_base = OTHER; note = Uracil
  modified_base 4169: mod_base = OTHER; note = Uracil
  modified_base 4175: mod_base = OTHER; note = Uracil
  modified_base 4182: mod_base = OTHER; note = Uracil
  modified_base 4184: mod_base = OTHER; note = Uracil
  modified_base 4187: mod_base = OTHER; note = Uracil
  modified_base 4191: mod_base = OTHER; note = Uracil
  modified_base 4195: mod_base = OTHER; note = Uracil
  modified_base 4203: mod_base = OTHER; note = Uracil
  modified_base 4211: mod_base = OTHER; note = Uracil
  modified_base 4213: mod_base = OTHER; note = Uracil
  modified_base 4215: mod_base = OTHER; note = Uracil
  modified_base 4216: mod_base = OTHER; note = Uracil
  modified_base 4217: mod_base = OTHER; note = Uracil
  modified_base 4219: mod_base = OTHER; note = Uracil
  modified_base 4226: mod_base = OTHER; note = Uracil
  modified_base 4230: mod_base = OTHER; note = Uracil
  modified_base 4231: mod_base = OTHER; note = Uracil
  modified_base 4240: mod_base = OTHER; note = Uracil
  modified_base 4241: mod_base = OTHER; note = Uracil
  modified_base 4242: mod_base = OTHER; note = Uracil
  modified_base 4246: mod_base = OTHER; note = Uracil
  modified_base 4250: mod_base = OTHER; note = Uracil
  modified_base 4265: mod_base = OTHER; note = Uracil
  modified_base 4270: mod_base = OTHER; note = Uracil
  modified_base 4280: mod_base = OTHER; note = Uracil
  modified_base 4284: mod_base = OTHER; note = Uracil
  modified_base 4285: mod_base = OTHER; note = Uracil
  modified_base 4291: mod_base = OTHER; note = Uracil
  modified_base 4294: mod_base = OTHER; note = Uracil
  modified_base 4296: mod_base = OTHER; note = Uracil
  modified_base 4297: mod_base = OTHER; note = Uracil
  modified_base 4305: mod_base = OTHER; note = Uracil
  modified_base 4306: mod_base = OTHER; note = Uracil
  modified_base 4308: mod_base = OTHER; note = Uracil
  modified_base 4309: mod_base = OTHER; note = Uracil
  modified_base 4310: mod_base = OTHER; note = Uracil
  modified_base 4319: mod_base = OTHER; note = Uracil
  modified_base 4325: mod_base = OTHER; note = Uracil
  modified_base 4328: mod_base = OTHER; note = Uracil
  modified_base 4331: mod_base = OTHER; note = Uracil
  modified_base 4334: mod_base = OTHER; note = Uracil
  modified_base 4340: mod_base = OTHER; note = Uracil
  modified_base 4344: mod_base = OTHER; note = Uracil
  modified_base 4350: mod_base = OTHER; note = Uracil
  modified_base 4354: mod_base = OTHER; note = Uracil
  modified_base 4361: mod_base = OTHER; note = Uracil
  modified_base 4367: mod_base = OTHER; note = Uracil
  modified_base 4368: mod_base = OTHER; note = Uracil
  modified_base 4369: mod_base = OTHER; note = Uracil
  modified_base 4373: mod_base = OTHER; note = Uracil
  modified_base 4381: mod_base = OTHER; note = Uracil
  modified_base 4383: mod_base = OTHER; note = Uracil
  modified_base 4393: mod_base = OTHER; note = Uracil
  modified_base 4398: mod_base = OTHER; note = Uracil
  modified_base 4407: mod_base = OTHER; note = Uracil
  modified_base 4410: mod_base = OTHER; note = Uracil
  modified_base 4413: mod_base = OTHER; note = Uracil
  modified_base 4428: mod_base = OTHER; note = Uracil
  modified_base 4431: mod_base = OTHER; note = Uracil
  modified_base 4433: mod_base = OTHER; note = Uracil
  modified_base 4436: mod_base = OTHER; note = Uracil
  modified_base 4437: mod_base = OTHER; note = Uracil
  modified_base 4441: mod_base = OTHER; note = Uracil
  modified_base 4446: mod_base = OTHER; note = Uracil
  modified_base 4449: mod_base = OTHER; note = Uracil
  modified_base 4454: mod_base = OTHER; note = Uracil
  modified_base 4464: mod_base = OTHER; note = Uracil
  modified_base 4465: mod_base = OTHER; note = Uracil
  modified_base 4470: mod_base = OTHER; note = Uracil
  modified_base 4472: mod_base = OTHER; note = Uracil
  modified_base 4478: mod_base = OTHER; note = Uracil
  modified_base 4479: mod_base = OTHER; note = Uracil
  modified_base 4480: mod_base = OTHER; note = Uracil
  modified_base 4482: mod_base = OTHER; note = Uracil
aaagaaaccaggagggaatcctaagttgatgcaatttgtcgctcacgataactttcacactctagaagtcaggaaagtcagatacctccacccaaagcaagtaacgtttcttttagcaggtctattgcttaacatyaaacaatttgtmaaagcaatcaaagagcgcaacaatgarttcaaaactgatatttttcttcgctctctgctctatcagcttcctctccacctcggagaccacatccacgatgatgtcaggaagtccatacttgctcctgaaccagagctttgtgcctggttctctttacaaacgggatatgctcccgcctccacctcaggccgtgttaacttacacgtgccaggaaccaagacctctcgcggaagaatcatacaacgatcttttgcgagcaatttctcagaaaagctcaagagatttccagaatgcctattcggtagccttgagtatttccagcgatttctatcaacatggactaaagacgttgaaagacgtatcttttctcgctgtcgagaaattcctytggggtctgacacgcttrtggagctcgctaatcttggcgagcttctccgcgttatggtggttggtgagcaatttcacaactcccgtcttctgtctcgccttgctgtacactgttacaagatttatggtgaagacggtttcatttctttttggaggattgccaatctggatcatttcgattgctttctcactcctgaagaaatccttttcagctcttcggtctacaccgaaatgtttgtatgaaaaggccatagacggtttcaagagtttcactatcccgcagagccccccaaaatcttgcgtgattcccatcacccacgcaagcggaaaccacgctggctacgccagctgtgtcaagctatacaacggcgaaaacgctttratgacggcaactcacgtyctacgtgactgccccaatgctgtagctatttccgccaaagggcttaagactcggattccactcgctgaattcaaaacaattgcgaaatccgacaaaggtgatgttaccctcctccgcggcccccccaaytgggaaggactgttaggctgyaaagcggccaacgtcattacagccgccaacctagcraaatgcaaagcatccatatactcttttgagagagatggctgggttagcggttaygccgagattgtrggctcagaaggtaccgatgttatggtcctgagccacacggaaggaggacactccggaagcccctactttaatggyaaaaccatyttaggggttcattcaggtgccagtgcyacaggaaattacaacttgatggcaccaatcccgyccmtccccggrctcacaagtccgacttatgtgtttgaaaccaccgcaccacaaggaagagttttcgcacaagaagatrtcgccsagatygaarryctctatgcgcaagcagtagagagatcgaatcgaaygaaaaatttcaagtcyaagactggaayaaattgggsygatatggakgaygatgaagacatcttcttcgaaagcagagaagatctgtcgggaaacggagtgcgcggcgccgaccgcgaaacaaacggagaaggcagctccaccccaaagacaaacaacgtcgatgggaaagagatgatggagaaaataatctcatctctggtggggaaaataaatctcgagaacatcgagaagaaagtgatagaggagatmtccgcgaaagcgttgaargctccaaagtcccgccgcagaagagccccaaagaaacagccggagagttcgaaagatacttctcctcactctacaactgggaggtaccaacctccccacgtgaggtccccggcttccgtcactgcggcaagctgccccaatactaccaccccaagcaaaaagaagaatccagctgggggaaaaccctcgtcgggaaccatcccgcgttgggtgaaaagacaagcggcttcggctggcccaagttcggccccgaagcggaactgaaragcctgcgaytgcaggcttcacggtggctggaacgcgcccagtccgcagaaataccctctgacgctgagagggagcgtgtgattcaaaagaccgcagatgtttaccatccttgccaaacaaatggacctgcggcaacccgaggaggarcactgacctggaacaacttcatgattgatttcaaacaggcrgtgttctcgctggagttcgatgccggaatcggcgtcccctatattgcctatggcaarcccacacaccgtgggtgggtagaagatcagacactccttccggtcctagctcaattraccttcatccgactacagaagatgttggaggttagtttcgaagatatgggaccwgaggagctgatccagaacggtttgtgcgaccccatccgattattcgtgaarggggagccccacaaacaagcgaagctcgatgaaggccgctaccgcctcatyatgagcgtttccctcgtggatcaaytggtrgcccgggttctgttccaaaaccagaacaagcgggaaattgccctttggagggccgtccccagtaaacccggtttyggcttgtccacggacgagcaagtgctggactttgtraaaggtttggcccgtcaagtaggcactackacaacagaggtagttgcyaattggaamaaytacttgacgcccacggattgctccggtttygactggagtgttgcggactggatgcttcacgacgatatgatcgtccgcaatagacttaccatcgacctcaaccctrttacagaaagattaagatcctgttggttgagrtgtgtttcaaactcagtgytatgcctgagtgatggtacccttttagcccaaacccatccgggcgttcagaaaagtgggagctacaatacttcaagctccaactcccggatcagagttatggccgcctttcayacaggtgctgcctgggctatggcgatgggcgatgacgccctcgagtccaatcccgctgacctagcagcgtacaaaagactaggtttcaaggttgaggtttccggacaactggaattctgctctcacatatttagagcgccggacctcgccctccctgtgaaygaaaayaaaatgatctacaaactgatctatggctacaatccagggagcggaaacgctgaggtagtttcaaactacttagccgcttgtttctcagttctgaacgagttgcggcaygatccagcgtccgttgaacttctttactcgtggttagtcgacccggtgctaccacaaaagataccaggagagtaaagaagagaagaagttagccctacatacgtaagttgcaagtaagggaaacaaagtcttacacccgagtataaacatagattacaagttcctagcaggcttttccgcaggcttcttgttaagcataccagtttcgacactcggattatacataatctacctgagaatttccacccacctgagatcaatcgttaatgaatacggtcgtgggtagaagaacaatcaatggaagaagacgaccacggaggcaarcacgacgcgctcagcgctctcagccagtggttgtggtccaagcctctcggacaacacaacgccgaccaagacgacgacgaagaggcggtaaccggacaggaagaactgttcctaccagaggagcaggttcgagcgagacatttgttttctcaaaagacaatctcgcgggaagttccagcggagcaatcacgttcgggccgagtctatcagactgcccggcattcgctaatggaatgctcaaggcctaccatgagtataaaatctcaatggtcattttggagttcatctccgaagcctcttcccaaaattccggttccatcgcttacgagctggacccacactgtaaactcaactccctttcctcaactatcaacaaattcgggatcacaaagcccgggaggagggcgtttacagcgtcttacatcaacgggacagaatggcacgacgttgccgaggaccaattcaggatcctctacaaaggcaatggttcttcatcgatagctggttctttcagaatcaccataaagtgccaattccacaatcccaaataggtagacgaggaacccggccccagcccagggccttctccctctccacaacccacaccccaaaagaaatatcgttttatcgtctatactggagtccccgtgacccgtataatggctcaatccacagatgacgccatctctttgtacgacatgccttcccaaaggtttcgctacatagaggacgagaacatgaactggacgaacctcgattcccgatggtattcccagaattctttgaaagccatcccgatgataatagtaccagtccctcaaggtgagtggacygtggaaatttcgatggaggggtatcaaccaacctcaagtaccacagatcctaataaggacaaacaagatggtctcattgcatacaatgatgacctcaaggagggttggaatgtgggggtttataataatgtggagataaccaacaataaggccgacaacactctyaagtacggccacccagacatggaactcaatagctgtcatttcaatcaaggacagtgtctggaaagagatggagatttgacttgtcacatcaaaacaactggtgatgacgcctccttctttattgttggacccgctgtycagaagcaatcaaagtacaattacgccgtttcatacggagcttggacagatcggatgatggagataggattgatagccatagcacttgatgaacaaggctcatccggttccacaargacagaaagaccaaagagagttgggcactccatggcagtctcaacctgggagactataaacttaccggagaaggaaaactccgagaaattcgaaaccagtcaaagacaagactctaaaactcaacctaaacaaggtttctcagatgctggttctgaagacaacaaggaatgggattttacacccgggactcgaatgtttcttcctgaagacttcgaggaaccccgtgaggttcagaatgacgatcttctcagagatagaggaattgtagtrgatccctccttgttggaaatacctgactcacctcctccgagagcggaccataaggacacagatccttgggctgaggttgaaagatataaatcttcccayaaagcccttgtcggcgaggacacgcgttcccgacaatcatccttracaggaggttcrcttgctggcggcagcctyagaaarctgaagccggttacaccggacgagtatgagcrggcraaamtgaaagaygagtcgagactccagtatgaacaggatctckctgagyatcacarragtttgaaaagacccggaagcgcgttaataccacgtgatgacggtgagctcatggagatggatccttctcaacgtaaattgatgaagaacgctggagtcaaccttgataagtatggtagaccagttaagaaaggtttcctctctttcaggaaatagtcccccagtacttaactggctcgttggtagtgatacgaggcgaaaatcaaaactacccttatccagctttatttctatttagctaataaagtcaagccagaga

SEQ ID NO: 7 - protein, 196 aa, synthetic construct
IRGRAPRTRPSPPPPLLPLLSLSLLLLSPTVRGQVTFAVSSLLLAVVAVSAYPASLNPESRAAILVQDSAPNADGSFKNNFQTENGIKQESVGYLKAGPEGPVAVFQGASAYVAPDGQTIQIGYIADENGYQPYGAHLPTPPPIPAEIQESLRYLASLPSTPEPKYQGGDRYIYGHTCLITLTVLHVMLSLIGYLT

SEQ ID NO: 8 - DNA, 591 bp, synthetic construct
  misc_feature 1..591: note = synthetic Polynucleotide
  source 1..591: mol_type = other DNA
ataagaggccgcgccccacgcacccgccctagtccccctcctcctttgttgccacttctttctctgtcccttctcctcctctctcctactgtgaggggtcaggtcacttttgccgtatcgtcgttgctgttagctgtcgtcgccgtcagcgcctaccccgcatcactgaacccggaatccagagccgccatcttggtccaagattcagcacccaacgccgatggatcattcaagaacaatttccaaaccgaaaacggaatcaaacaagaatcagtcggatacttgaaggctggcccagaaggacccgtagctgtgttccagggagcttctgcgtacgtcgccccagacggccagaccatccaaatcggatacatcgccgacgagaacggttaccagccgtacggcgctcatttgcccactccaccaccaatcccagctgagatccaagagtcgctcagatacctcgcctctctgcccagcacccccgaaccaaaataccagggaggagaccggtacatatacgggcatacctgcttgataactctgacagtgctgcatgtgatgctttcacttattggttacttgacttga

SEQ ID NO: 9 - RNA, 591 nt, synthetic construct
  misc_feature 1..591: note = synthetic Polynucleotide
  source 1..591: mol_type = other RNA
  modified_base 2: mod_base = OTHER; note = Uracil
  modified_base 30: mod_base = OTHER; note = Uracil
  modified_base 33: mod_base = OTHER; note = Uracil
  modified_base 39: mod_base = OTHER; note = Uracil
  modified_base 42: mod_base = OTHER; note = Uracil
  modified_base 45: mod_base = OTHER; note = Uracil
  modified_base 46: mod_base = OTHER; note = Uracil
  modified_base 47: mod_base = OTHER; note = Uracil
  modified_base 49: mod_base = OTHER; note = Uracil
  modified_base 50: mod_base = OTHER; note = Uracil
  modified_base 56: mod_base = OTHER; note = Uracil
  modified_base 57: mod_base = OTHER; note = Uracil
  modified_base 59: mod_base = OTHER; note = Uracil
  modified_base 60: mod_base = OTHER; note = Uracil
  modified_base 61: mod_base = OTHER; note = Uracil
  modified_base 63: mod_base = OTHER; note = Uracil
  modified_base 65: mod_base = OTHER; note = Uracil
  modified_base 67: mod_base = OTHER; note = Uracil
  modified_base 71: mod_base = OTHER; note = Uracil
  modified_base 72: mod_base = OTHER; note = Uracil
  modified_base 74: mod_base = OTHER; note = Uracil
  modified_base 77: mod_base = OTHER; note = Uracil
  modified_base 80: mod_base = OTHER; note = Uracil
  modified_base 82: mod_base = OTHER; note = Uracil
  modified_base 84: mod_base = OTHER; note = Uracil
  modified_base 87: mod_base = OTHER; note = Uracil
  modified_base 90: mod_base = OTHER; note = Uracil
  modified_base 92: mod_base = OTHER; note = Uracil
  modified_base 99: mod_base = OTHER; note = Uracil
  modified_base 104: mod_base = OTHER; note = Uracil
  modified_base 108: mod_base = OTHER; note = Uracil
  modified_base 109: mod_base = OTHER; note = Uracil
  modified_base 110: mod_base = OTHER; note = Uracil
  modified_base 111: mod_base = OTHER; note = Uracil
  modified_base 116: mod_base = OTHER; note = Uracil
  modified_base 118: mod_base = OTHER; note = Uracil
  modified_base 121: mod_base = OTHER; note = Uracil
  modified_base 124: mod_base = OTHER; note = Uracil
  modified_base 125: mod_base = OTHER; note = Uracil
  modified_base 128: mod_base = OTHER; note = Uracil
  modified_base 130: mod_base = OTHER; note = Uracil
  modified_base 131: mod_base = OTHER; note = Uracil
  modified_base 135: mod_base = OTHER; note = Uracil
  modified_base 137: mod_base = OTHER; note = Uracil
  modified_base 140: mod_base = OTHER; note = Uracil
  modified_base 146: mod_base = OTHER; note = Uracil
  modified_base 154: mod_base = OTHER; note = Uracil
  modified_base 163: mod_base = OTHER; note = Uracil
  modified_base 167: mod_base = OTHER; note = Uracil
  modified_base 178: mod_base = OTHER; note = Uracil
  modified_base 191: mod_base = OTHER; note = Uracil
  modified_base 193: mod_base = OTHER; note = Uracil
  modified_base 194: mod_base = OTHER; note = Uracil
  modified_base 197: mod_base = OTHER; note = Uracil
  modified_base 204: mod_base = OTHER; note = Uracil
  modified_base 205: mod_base = OTHER; note = Uracil
  modified_base 222: mod_base = OTHER; note = Uracil
  modified_base 226: mod_base = OTHER; note = Uracil
  modified_base 229: mod_base = OTHER; note = Uracil
  modified_base 230: mod_base = OTHER; note = Uracil
  modified_base 240: mod_base = OTHER; note = Uracil
  modified_base 241: mod_base = OTHER; note = Uracil
  modified_base 242: mod_base = OTHER; note = Uracil
  modified_base 260: mod_base = OTHER; note = Uracil
  modified_base 271: mod_base = OTHER; note = Uracil
  modified_base 275: mod_base = OTHER; note = Uracil
  modified_base 280: mod_base = OTHER; note = Uracil
  modified_base 283: mod_base = OTHER; note = Uracil
  modified_base 284: mod_base = OTHER; note = Uracil
  modified_base 291: mod_base = OTHER; note = Uracil
  modified_base 308: mod_base = OTHER; note = Uracil
  modified_base 312: mod_base = OTHER; note = Uracil
  modified_base 314: mod_base = OTHER; note = Uracil
  modified_base 316: mod_base = OTHER; note = Uracil
  modified_base 317: mod_base = OTHER; note = Uracil
  modified_base 327: mod_base = OTHER; note = Uracil
  modified_base 328: mod_base = OTHER; note = Uracil
  modified_base 330: mod_base = OTHER; note = Uracil
  modified_base 334: mod_base = OTHER; note = Uracil
  modified_base 338: mod_base = OTHER; note = Uracil
  modified_base 359: mod_base = OTHER; note = Uracil
  modified_base 365: mod_base = OTHER; note = Uracil
  modified_base 370: mod_base = OTHER; note = Uracil
  modified_base 374: mod_base = OTHER; note = Uracil
  modified_base 390: mod_base = OTHER; note = Uracil
  modified_base 391: mod_base = OTHER; note = Uracil
  modified_base 400: mod_base = OTHER; note = Uracil
  modified_base 408: mod_base = OTHER; note = Uracil
  modified_base 411: mod_base = OTHER; note = Uracil
  modified_base 412: mod_base = OTHER; note = Uracil
  modified_base 413: mod_base = OTHER; note = Uracil
  modified_base 420: mod_base = OTHER; note = Uracil
  modified_base 431: mod_base = OTHER; note = Uracil
  modified_base 438: mod_base = OTHER; note = Uracil
  modified_base 443: mod_base = OTHER; note = Uracil
  modified_base 451: mod_base = OTHER; note = Uracil
  modified_base 455: mod_base = OTHER; note = Uracil
  modified_base 460: mod_base = OTHER; note = Uracil
  modified_base 464: mod_base = OTHER; note = Uracil
  modified_base 469: mod_base = OTHER; note = Uracil
  modified_base 471: mod_base = OTHER; note = Uracil
  modified_base 473: mod_base = OTHER; note = Uracil
  modified_base 496: mod_base = OTHER; note = Uracil
  modified_base 514: mod_base = OTHER; note = Uracil
  modified_base 518: mod_base = OTHER; note = Uracil
  modified_base 520: mod_base = OTHER; note = Uracil
  modified_base 528: mod_base = OTHER; note = Uracil
  modified_base 532: mod_base = OTHER; note = Uracil
  modified_base 535: mod_base = OTHER; note = Uracil
  modified_base 536: mod_base = OTHER; note = Uracil
  modified_base 539: mod_base = OTHER; note = Uracil
  modified_base 543: mod_base = OTHER; note = Uracil
  modified_base 545: mod_base = OTHER; note = Uracil
  modified_base 551: mod_base = OTHER; note = Uracil
  modified_base 554: mod_base = OTHER; note = Uracil
  modified_base 558: mod_base = OTHER; note = Uracil
  modified_base 560: mod_base = OTHER; note = Uracil
  modified_base 563: mod_base = OTHER; note = Uracil
  modified_base 566: mod_base = OTHER; note = Uracil
  modified_base 567: mod_base = OTHER; note = Uracil
  modified_base 568: mod_base = OTHER; note = Uracil
  modified_base 572: mod_base = OTHER; note = Uracil
  modified_base 573: mod_base = OTHER; note = Uracil
  modified_base 575: mod_base = OTHER; note = Uracil
  modified_base 576: mod_base = OTHER; note = Uracil
  modified_base 579: mod_base = OTHER; note = Uracil
  modified_base 580: mod_base = OTHER; note = Uracil
  modified_base 583: mod_base = OTHER; note = Uracil
  modified_base 584: mod_base = OTHER; note = Uracil
  modified_base 588: mod_base = OTHER; note = Uracil
  modified_base 589: mod_base = OTHER; note = Uracil
ataagaggccgcgccccacgcacccgccctagtccccctcctcctttgttgccacttctttctctgtcccttctcctcctctctcctactgtgaggggtcaggtcacttttgccgtatcgtcgttgctgttagctgtcgtcgccgtcagcgcctaccccgcatcactgaacccggaatccagagccgccatcttggtccaagattcagcacccaacgccgatggatcattcaagaacaatttccaaaccgaaaacggaatcaaacaagaatcagtcggatacttgaaggctggcccagaaggacccgtagctgtgttccagggagcttctgcgtacgtcgccccagacggccagaccatccaaatcggatacatcgccgacgagaacggttaccagccgtacggcgctcatttgcccactccaccaccaatcccagctgagatccaagagtcgctcagatacctcgcctctctgcccagcacccccgaaccaaaataccagggaggagaccggtacatatacgggcatacctgcttgataactctgacagtgctgcatgtgatgctttcacttattggttacttgacttga

SEQ ID NO: 10 - protein, 297 aa, synthetic construct
MIRGRAPRTRPSPPPPLLPLLSLSLLLLSPTVRGSQVFVDFFAKTHLMTRLLVSVACPAILARDSWYASPATGPIDKYDPEQPPSMVIIHHSRLPPCSTTESCIVRMLELQRLHQRDRHWFDIGFNFAIGGDGSVYEGRGWNQKPAAVKNYNNKSINIAFLGDFSSSVPSAEMLKTARDLIDCGVRTGKISRDYKLVGYSEQDVSSSVSGPSSSTSSPTSDSSDSSSSPSSFPSSLFSHLQSWPHWSPMNLTDQAEPPTELKLALLVEGGDRYIYGHTCLITLTVLHVMLSLIGYLT

SEQ ID NO: 11 - DNA, 894 bp, synthetic construct
  misc_feature 1..894: note = synthetic Polynucleotide
  source 1..894: mol_type = other DNA
atgataagaggccgcgccccacgcacccgccctagtccccctcctcctttgttgccacttctttctctgtcccttctcctcctctctcctactgtgaggggtagccaggttttcgtggatttctttgcgaagacgcacctcatgacccggctgctagtctcggtggcatgtccggcgatcctagcccgagattcgtggtacgcgagccctgcgacgggcccgatcgacaaatacgaccccgagcaacctccatccatggtcatcatccaccactcccgacttccaccttgcagcaccaccgaaagttgtatcgtacgaatgctagaattgcagaggctacaccagagagatcgccactggttcgacatcggtttcaattttgcgataggtggcgatggatctgtttatgagggacgaggatggaatcaaaaaccagcggctgttaaaaattacaataacaaaagcatcaacatagcatttctcggcgattttagctcgagcgttccctcggcggagatgctgaaaacggcgcgggacctaatcgactgcggcgtgcgaaccgggaaaatctcgcgggactacaagctcgtgggctactcggagcaggacgtgagctcgagcgtgagcgggccgagcagctcgacgtcatcgccgacgtcagactcgtccgactcctcgtcatcgccgtcgtcattcccgtcctccctgttcagtcacctccagagctggccgcactggtccccgatgaacctcaccgaccaggccgagccgcccaccgagctcaaactcgccctcctcgtcgaaggaggagaccggtacatatacgggcatacctgcttgataactctgacagtgctgcatgtgatgctttcacttattggttacttgacttga

SEQ ID NO: 12 - skipped

SEQ ID NO: 13 - protein, 682 aa, synthetic construct
MIRGRAPRTRPSPPPPLLPLLSLSLLLLSPTVRGHKYHQHHCDDSSHHHHRHNHHHHLNRHHRCCLAAVFLLLAVAIVSPGRAEHVVLLDTTTEPSLRWTTYPYGPDANAAGWVEESYINFEKGINWRSYVVCDVTKQNVNNWVWTPFIERGLANLIYIEVKFTIRDCSLFPGYALSCKETFSLLYYEFDVATREPPPWEPDSYKSVGRIAAGEGRFNANNEVVINTETKAVKVTKKGVYFAFRDQGACISIMAVKVYYIVCPEVVINFANFSATPTARELTQIEHATGKCVDNAEVVGGGAPTYLCKGDGKWYLPSGGCKCKAGFEADIEAQTCIICPPGKYKYGVGDDKCQPCPAHSKAPDQGMSECRCNTGYYRSPKDPKSVPCTQPPSAPQNLTVNFVDQSTVTLSWNPPNFLGGRTDIVYRVTCDMCGPSVVFMPNNEVFNDTKITISGLGPVTTYKFHVWAENGVSNLTSSENRQFVDIAVTTTEASVKSASVNNVRVMLVKASEITLSWDPPMASFFDSGDDDAAVEVYEVKFYPRGDESNVSNKLTADRHMVFTALRPKTDYGFQVRAKTAHGWGEYSPTIYKTTGQLLGSGKNTQQQVQRKNGIKDDTAYIGDEDNMEVRIIAGATVAVVVVLVVVIIMTVLFLGGDRYIYGHTCLITLTVLHVMLSLIGYLT

SEQ ID NO: 14 - DNA, 2049 bp, synthetic construct
  misc_feature 1..2049: note = synthetic Polynucleotide
  source 1..2049: mol_type = other DNA
atgataagaggccgcgccccacgcacccgccctagtccccctcctcctttgttgccacttctttctctgtcccttctcctcctctctcctactgtgaggggtcacaagtatcaccaacaccactgcgacgacagcagccatcaccaccatcggcacaaccaccatcatcacctcaaccgtcaccaccgatgctgtctcgccgcggtgtttctgctgctggccgtcgcgatcgtcagccccggacgagccgaacacgttgtacttttggacaccacgacagagcctagtctacggtggactacgtacccttacggaccggacgccaacgcagcagggtgggtcgaagagagctacataaattttgaaaagggcatcaactggcggtcgtacgtcgtgtgcgatgtcaccaagcaaaacgtcaacaattgggtgtggacgcctttcatagagcgcgggctggccaatctcatttacatcgaagtcaagtttaccatccgagattgttcgctgttcccggggtacgcgctgtcatgcaaagaaacgttttcactactctactatgagtttgacgtggccaccagggaaccaccgccttgggaacctgacagctataaatctgttggtcggatagctgctggtgaagggaggttcaacgctaataacgaagtggtgatcaatactgagacaaaagctgtgaaagttaccaaaaaaggagtgtactttgccttcagggaccagggcgcatgtatttccatcatggcagtcaaggtgtattacatcgtgtgcccagaagttgtgataaactttgcaaacttctctgctactccaactgctagagaacttacccaaatcgaacatgccaccggcaaatgtgttgataatgctgaagtggtaggaggtggtgctccaacttacctgtgcaaaggagatggaaaatggtatctaccgtcaggtggatgcaagtgcaaagctggttttgaagcagacatagaagcgcaaacttgtataatctgtcctccgggcaagtacaaatacggcgttggggacgacaagtgtcagccgtgtccggcgcacagcaaagcgcccgaccaggggatgtccgagtgcaggtgcaacaccggatactacaggtcgcccaaggacccaaaatcggtgccgtgcacgcaaccgccgtcagcgccgcaaaacttgacagtcaactttgtggaccagtcgactgtgaccctgtcgtggaacccaccgaacttcctcggtggcaggaccgatatcgtgtacagagtgacatgcgacatgtgcggtccatccgttgtgttcatgcccaacaacgaggtgttcaacgataccaaaataacgatcagcggtctgggtccggtcaccacgtacaagtttcacgtatgggccgaaaacggtgtcagtaatctaacgtcgtccgaaaaccggcaattcgtcgacatagccgtgaccactacagaagcttccgttaagtctgcgtccgtcaacaatgtccgtgtcatgttggtaaaggcgtccgagatcacgctatcttgggatccgcccatggcgagcttcttcgattctggcgatgacgacgctgccgtcgaagtttacgaagtgaagttctatccccgaggtgacgagtcaaacgtcagcaacaaacttaccgccgacaggcacatggtgtttacggcactcaggcccaagacggactacggattccaagtgcgagcgaagaccgctcacggctggggcgagtacagtccgactatttacaaaacgacggggcaattgctcggaagcggtaagaacacacaacaacaagtccaaagaaaaaacgggattaaagatgatacggcctacatcggtgacgaagacaacatggaagtccgaataatcgcaggtgcgacggtcgcggtagtggtggtcttagtagttgtcatcatcatgacggtattgttcttaggaggagaccggtacatatacgggcatacctgcttgataactctgacagtgctgcatgtgatgctttcacttattggttacttgacttga

SEQ ID NO: 15 - RNA, 2049 nt, synthetic construct
  misc_feature 1..2049: note = synthetic Polynucleotide
  source 1..2049: mol_type = other RNA
  modified_base 2: mod_base = OTHER; note = Uracil
  modified_base 5: mod_base = OTHER; note = Uracil
  modified_base 33: mod_base = OTHER; note = Uracil
  modified_base 36: mod_base = OTHER; note = Uracil
  modified_base 42: mod_base = OTHER; note = Uracil
  modified_base 45: mod_base = OTHER; note = Uracil
  modified_base 48: mod_base = OTHER; note = Uracil
  modified_base 49: mod_base = OTHER; note = Uracil
  modified_base 50: mod_base = OTHER; note = Uracil
  modified_base 52: mod_base = OTHER; note = Uracil
  modified_base 53: mod_base = OTHER; note = Uracil
  modified_base 59: mod_base = OTHER; note = Uracil
  modified_base 60: mod_base = OTHER; note = Uracil
  modified_base 62: mod_base = OTHER; note = Uracil
  modified_base 63: mod_base = OTHER; note = Uracil
  modified_base 64: mod_base = OTHER; note = Uracil
  modified_base 66: mod_base = OTHER; note = Uracil
  modified_base 68: mod_base = OTHER; note = Uracil
  modified_base 70: mod_base = OTHER; note = Uracil
  modified_base 74: mod_base = OTHER; note = Uracil
  modified_base 75: mod_base = OTHER; note = Uracil
  modified_base 77: mod_base = OTHER; note = Uracil
  modified_base 80: mod_base = OTHER; note = Uracil
  modified_base 83: mod_base = OTHER; note = Uracil
  modified_base 85: mod_base = OTHER; note = Uracil
  modified_base 87: mod_base = OTHER; note = Uracil
  modified_base 90: mod_base = OTHER; note = Uracil
  modified_base 93: mod_base = OTHER; note = Uracil
  modified_base 95: mod_base = OTHER; note = Uracil
  modified_base 102: mod_base = OTHER; note = Uracil
  modified_base 109: mod_base = OTHER; note = Uracil
  modified_base 111: mod_base = OTHER; note = Uracil
  modified_base 124: mod_base = OTHER; note = Uracil
  modified_base 141: mod_base = OTHER; note = Uracil
  modified_base 150: mod_base = OTHER; note = Uracil
  modified_base 165: mod_base = OTHER; note = Uracil
  modified_base 168: mod_base = OTHER; note = Uracil
  modified_base 173: mod_base = OTHER; note = Uracil
  modified_base 180: mod_base = OTHER; note = Uracil
  modified_base 190: mod_base = OTHER; note = Uracil
  modified_base 193: mod_base = OTHER; note = Uracil
  modified_base 195: mod_base = OTHER; note = Uracil
  modified_base 197: mod_base = OTHER; note = Uracil
  modified_base 206: mod_base = OTHER; note = Uracil
  modified_base 208: mod_base = OTHER; note = Uracil
  modified_base 209: mod_base = OTHER; note = Uracil
  modified_base 210: mod_base = OTHER; note = Uracil
  modified_base 212: mod_base = OTHER; note = Uracil
  modified_base 215: mod_base = OTHER; note = Uracil
  modified_base 218: mod_base = OTHER; note = Uracil
  modified_base 224: mod_base = OTHER; note = Uracil
  modified_base 230: mod_base = OTHER; note = Uracil
  modified_base 233: mod_base = OTHER; note = Uracil
  modified_base 257: mod_base = OTHER; note = Uracil
  modified_base 258: mod_base = OTHER; note = Uracil
  modified_base 260: mod_base = OTHER; note = Uracil
  modified_base 263: mod_base = OTHER; note = Uracil
  modified_base 264: mod_base = OTHER; note = Uracil
  modified_base 265: mod_base = OTHER; note = Uracil
  modified_base 266: mod_base = OTHER; note = Uracil
  modified_base 285: mod_base = OTHER; note = Uracil
  modified_base 288: mod_base = OTHER; note = Uracil
  modified_base 290: mod_base = OTHER; note = Uracil
  modified_base 295: mod_base = OTHER; note = Uracil
  modified_base 300: mod_base = OTHER; note = Uracil
  modified_base 304: mod_base = OTHER; note = Uracil
  modified_base 309: mod_base = OTHER; note = Uracil
  modified_base 310: mod_base = OTHER; note = Uracil
  modified_base 337: mod_base = OTHER; note = Uracil
  modified_base 341: mod_base = OTHER; note = Uracil
  modified_base 352: mod_base = OTHER; note = Uracil
  modified_base 356: mod_base = OTHER; note = Uracil
  modified_base 360: mod_base = OTHER; note = Uracil
  modified_base 361: mod_base = OTHER; note = Uracil
  modified_base 362: mod_base = OTHER; note = Uracil
  modified_base 363: mod_base = OTHER; note = Uracil
  modified_base 374: mod_base = OTHER; note = Uracil
  modified_base 379: mod_base = OTHER; note = Uracil
  modified_base 385: mod_base = OTHER; note = Uracil
  modified_base 388: mod_base = OTHER; note = Uracil
  modified_base 392: mod_base = OTHER; note = Uracil
  modified_base 395: mod_base = OTHER; note = Uracil
  modified_base 397: mod_base = OTHER; note = Uracil
  modified_base 402: mod_base = OTHER; note = Uracil
  modified_base 404: mod_base = OTHER; note = Uracil
  modified_base 419: mod_base = OTHER; note = Uracil
  modified_base 426: mod_base = OTHER; note = Uracil
  modified_base 427: mod_base = OTHER; note = Uracil
  modified_base 431: mod_base = OTHER; note = Uracil
  modified_base 433: mod_base = OTHER; note = Uracil
  modified_base 441: mod_base = OTHER; note = Uracil
  modified_base 442: mod_base = OTHER; note = Uracil
  modified_base 443: mod_base = OTHER; note = Uracil
  modified_base 446: mod_base = OTHER; note = Uracil
  modified_base 458: mod_base = OTHER; note = Uracil
  modified_base 465: mod_base = OTHER; note = Uracil
  modified_base 467: mod_base = OTHER; note = Uracil
  modified_base 470: mod_base = OTHER; note = Uracil
  modified_base 471: mod_base = OTHER; note = Uracil
  modified_base 472: mod_base = OTHER; note = Uracil
  modified_base 476: mod_base = OTHER; note = Uracil
  modified_base 482: mod_base = OTHER; note = Uracil
  modified_base 487: mod_base = OTHER; note = Uracil
  modified_base 488: mod_base = OTHER; note = Uracil
  modified_base 489: mod_base = OTHER; note = Uracil
  modified_base 494: mod_base = OTHER; note = Uracil
  modified_base 501: mod_base = OTHER; note = Uracil
  modified_base 502: mod_base = OTHER; note = Uracil
  modified_base 504: mod_base = OTHER; note = Uracil
  modified_base 505: mod_base = OTHER; note = Uracil
  modified_base 509: mod_base = OTHER; note = Uracil
  modified_base 511: mod_base = OTHER; note = Uracil
  modified_base 512: mod_base = OTHER; note = Uracil
  modified_base 520: mod_base = OTHER; note = Uracil
  modified_base 527: mod_base = OTHER; note = Uracil
  modified_base 529: mod_base = OTHER; note = Uracil
  modified_base 532: mod_base = OTHER; note = Uracil
  modified_base 544: mod_base = OTHER; note = Uracil
  modified_base 545: mod_base = OTHER; note = Uracil
  modified_base 546: mod_base = OTHER; note = Uracil
  modified_base 547: mod_base = OTHER; note = Uracil
  modified_base 551: mod_base = OTHER; note = Uracil
  modified_base 554: mod_base = OTHER; note = Uracil
  modified_base 556: mod_base = OTHER; note = Uracil
  modified_base 559: mod_base = OTHER; note = Uracil
  modified_base 561: mod_base = OTHER; note = Uracil
  modified_base 565: mod_base = OTHER; note = Uracil
  modified_base 566: mod_base = OTHER; note = Uracil
  modified_base 567: mod_base = OTHER; note = Uracil
  modified_base 572: mod_base = OTHER; note = Uracil
  modified_base 594: mod_base = OTHER; note = Uracil
  modified_base 595: mod_base = OTHER; note = Uracil
  modified_base 603: mod_base = OTHER; note = Uracil
  modified_base 610: mod_base = OTHER; note = Uracil
  modified_base 612: mod_base = OTHER; note = Uracil
  modified_base 616: mod_base = OTHER; note = Uracil
  modified_base 618: mod_base = OTHER; note = Uracil
  modified_base 620: mod_base = OTHER; note = Uracil
  modified_base 621: mod_base = OTHER; note = Uracil
  modified_base 624: mod_base = OTHER; note = Uracil
  modified_base 629: mod_base = OTHER; note = Uracil
  modified_base 633: mod_base = OTHER; note = Uracil
  modified_base 636: mod_base = OTHER; note = Uracil
  modified_base 639: mod_base = OTHER; note = Uracil
  modified_base 649: mod_base = OTHER; note = Uracil
  modified_base 650: mod_base = OTHER; note = Uracil
  modified_base 657: mod_base = OTHER; note = Uracil
  modified_base 660: mod_base = OTHER; note = Uracil
  modified_base 668: mod_base = OTHER; note = Uracil
  modified_base 671: mod_base = OTHER; note = Uracil
  modified_base 674: mod_base = OTHER; note = Uracil
  modified_base 678: mod_base = OTHER; note = Uracil
  modified_base 681: mod_base = OTHER; note = Uracil
  modified_base 693: mod_base = OTHER; note = Uracil
  modified_base 695: mod_base = OTHER; note = Uracil
  modified_base 701: mod_base = OTHER; note = Uracil
  modified_base 702: mod_base = OTHER; note = Uracil
  modified_base 716: mod_base = OTHER; note = Uracil
  modified_base 718: mod_base = OTHER; note = Uracil
  modified_base 721: mod_base = OTHER; note = Uracil
  modified_base 722: mod_base = OTHER; note = Uracil
  modified_base 723: mod_base = OTHER; note = Uracil
  modified_base 727: mod_base = OTHER; note = Uracil
  modified_base 728: mod_base = OTHER; note = Uracil
  modified_base 745: mod_base = OTHER; note = Uracil
  modified_base 747: mod_base = OTHER; note = Uracil
  modified_base 749: mod_base = OTHER; note = Uracil
  modified_base 750: mod_base = OTHER; note = Uracil
  modified_base 751: mod_base = OTHER; note = Uracil
  modified_base 755: mod_base = OTHER; note = Uracil
  modified_base 758: mod_base = OTHER; note = Uracil
  modified_base 764: mod_base = OTHER; note = Uracil
  modified_base 770: mod_base = OTHER; note = Uracil
  modified_base 772: mod_base = OTHER; note = Uracil
  modified_base 774: mod_base = OTHER; note = Uracil
  modified_base 775: mod_base = OTHER; note = Uracil
  modified_base 779: mod_base = OTHER; note = Uracil
  modified_base 782: mod_base = OTHER; note = Uracil
  modified_base 784: mod_base = OTHER; note = Uracil
  modified_base 794: mod_base = OTHER; note = Uracil
  modified_base 795: mod_base = OTHER; note = Uracil
  modified_base 797: mod_base = OTHER; note = Uracil
  modified_base 800: mod_base = OTHER; note = Uracil
  modified_base 805: mod_base = OTHER; note = Uracil
  modified_base 806: mod_base = OTHER; note = Uracil
  modified_base 807: mod_base = OTHER; note = Uracil
  modified_base 814: mod_base = OTHER; note = Uracil
  modified_base 815: mod_base = OTHER; note = Uracil
  modified_base 817: mod_base = OTHER; note = Uracil
  modified_base 819: mod_base = OTHER; note = Uracil
  modified_base 822: mod_base = OTHER; note = Uracil
  modified_base 825: mod_base = OTHER; note = Uracil
  modified_base 831: mod_base = OTHER; note = Uracil
  modified_base 834: mod_base = OTHER; note = Uracil
  modified_base 842: mod_base = OTHER; note = Uracil
  modified_base 843: mod_base = OTHER; note = Uracil
  modified_base 851: mod_base = OTHER; note = Uracil
  modified_base 858: mod_base = OTHER; note = Uracil
  modified_base 871: mod_base = OTHER; note = Uracil
  modified_base 873: mod_base = OTHER; note = Uracil
  modified_base 875: mod_base = OTHER; note = Uracil
  modified_base 876: mod_base = OTHER; note = Uracil
  modified_base 879: mod_base = OTHER; note = Uracil
  modified_base 882: mod_base = OTHER; note = Uracil
  modified_base 885: mod_base = OTHER; note = Uracil
  modified_base 890: mod_base = OTHER; note = Uracil
  modified_base 893: mod_base = OTHER; note = Uracil
  modified_base 900: mod_base = OTHER; note = Uracil
  modified_base 903: mod_base = OTHER; note = Uracil
  modified_base 906: mod_base = OTHER; note = Uracil
  modified_base 912: mod_base = OTHER; note = Uracil
  modified_base 913: mod_base = OTHER; note = Uracil
  modified_base 917: mod_base = OTHER; note = Uracil
  modified_base 919: mod_base = OTHER; note = Uracil
  modified_base 930: mod_base = OTHER; note = Uracil
  modified_base 937: mod_base = OTHER; note = Uracil
  modified_base 940: mod_base = OTHER; note = Uracil
  modified_base 942: mod_base = OTHER; note = Uracil
  modified_base 944: mod_base = OTHER; note = Uracil
  modified_base 949: mod_base = OTHER; note = Uracil
  modified_base 954: mod_base = OTHER; note = Uracil
  modified_base 958: mod_base = OTHER; note = Uracil
  modified_base 964: mod_base = OTHER; note = Uracil
  modified_base 972: mod_base = OTHER; note = Uracil
  modified_base 975: mod_base = OTHER; note = Uracil
  modified_base 976: mod_base = OTHER; note = Uracil
  modified_base 977: mod_base = OTHER; note = Uracil
  modified_base 978: mod_base = OTHER; note = Uracil
  modified_base 989: mod_base = OTHER; note = Uracil
  modified_base 1002: mod_base = OTHER; note = Uracil
  modified_base 1003: mod_base = OTHER; note = Uracil
  modified_base 1005: mod_base = OTHER; note = Uracil
  modified_base 1007: mod_base = OTHER; note = Uracil
  modified_base 1010: mod_base = OTHER; note = Uracil
  modified_base 1012: mod_base = OTHER; note = Uracil
  modified_base 1014: mod_base = OTHER; note = Uracil
  modified_base 1017: mod_base = OTHER; note = Uracil
  modified_base 1027: mod_base = OTHER; note = Uracil
  modified_base 1033: mod_base = OTHER; note = Uracil
  modified_base 1040: mod_base = OTHER; note = Uracil
  modified_base 1041: mod_base = OTHER; note = Uracil
  modified_base 1054: mod_base = OTHER; note = Uracil
  modified_base 1056: mod_base = OTHER; note = Uracil
  modified_base 1063: mod_base = OTHER; note = Uracil
  modified_base 1065: mod_base = OTHER; note = Uracil
  modified_base 1097: mod_base = OTHER; note = Uracil
  modified_base 1099: mod_base = OTHER; note = Uracil
  modified_base 1105: mod_base = OTHER; note = Uracil
  modified_base 1111: mod_base = OTHER; note = Uracil
  modified_base 1123: mod_base = OTHER; note = Uracil
  modified_base 1126: mod_base = OTHER; note = Uracil
  modified_base 1132: mod_base = OTHER; note = Uracil
  modified_base 1150: mod_base = OTHER; note = Uracil
  modified_base 1154: mod_base = OTHER; note = Uracil
  modified_base 1159: mod_base = OTHER; note = Uracil
  modified_base 1174: mod_base = OTHER; note = Uracil
  modified_base 1189: mod_base = OTHER; note = Uracil
  modified_base 1190: mod_base = OTHER; note = Uracil
  modified_base 1196: mod_base = OTHER; note = Uracil
  modified_base 1201: mod_base = OTHER; note = Uracil
  modified_base 1202: mod_base = OTHER; note = Uracil
  modified_base 1203: mod_base = OTHER; note = Uracil
  modified_base 1205: mod_base = OTHER; note = Uracil
  modified_base 1213: mod_base = OTHER; note = Uracil
  modified_base 1218: mod_base = OTHER; note = Uracil
  modified_base 1220: mod_base = OTHER; note = Uracil
  modified_base 1226: mod_base = OTHER; note = Uracil
  modified_base 1228: mod_base = OTHER; note = Uracil
  modified_base 1231: mod_base = OTHER; note = Uracil
  modified_base 1246: mod_base = OTHER; note = Uracil
  modified_base 1247: mod_base = OTHER; note = Uracil
  modified_base 1250: mod_base = OTHER; note = Uracil
  modified_base 1254: mod_base = OTHER; note = Uracil
  modified_base 1266: mod_base = OTHER; note = Uracil
  modified_base 1268: mod_base = OTHER; note = Uracil
  modified_base 1271: mod_base = OTHER; note = Uracil
  modified_base 1273: mod_base = OTHER; note = Uracil
  modified_base 1280: mod_base = OTHER; note = Uracil
  modified_base 1285: mod_base = OTHER; note = Uracil
  modified_base 1292: mod_base = OTHER; note = Uracil
  modified_base 1294: mod_base = OTHER; note = Uracil
  modified_base 1299: mod_base = OTHER; note = Uracil
  modified_base 1303: mod_base = OTHER; note = Uracil
  modified_base 1307: mod_base = OTHER; note = Uracil
  modified_base 1308: mod_base = OTHER; note = Uracil
  modified_base 1310: mod_base = OTHER; note = Uracil
  modified_base 1312: mod_base = OTHER; note = Uracil
  modified_base 1313: mod_base = OTHER; note = Uracil
  modified_base 1316: mod_base = OTHER; note = Uracil
  modified_base 1331: mod_base = OTHER; note = Uracil
  modified_base 1333: mod_base = OTHER; note = Uracil
  modified_base 1334: mod_base = OTHER; note = Uracil
  modified_base 1341: mod_base = OTHER; note = Uracil
  modified_base 1349: mod_base = OTHER; note = Uracil
  modified_base 1355: mod_base = OTHER; note = Uracil
  modified_base 1362: mod_base = OTHER; note = Uracil
  modified_base 1364: mod_base = OTHER; note = Uracil
  modified_base 1368: mod_base = OTHER; note = Uracil
  modified_base 1373: mod_base = OTHER; note = Uracil
  modified_base 1381: mod_base = OTHER; note = Uracil
  modified_base 1387: mod_base = OTHER; note = Uracil
  modified_base 1388: mod_base = OTHER; note = Uracil
  modified_base 1389: mod_base = OTHER; note = Uracil
  modified_base 1394: mod_base = OTHER; note = Uracil
  modified_base 1396: mod_base = OTHER; note = Uracil
  modified_base 1410: mod_base = OTHER; note = Uracil
  modified_base 1412: mod_base = OTHER; note = Uracil
  modified_base 1416: mod_base = OTHER; note = Uracil
  modified_base 1419: mod_base = OTHER; note = Uracil
  modified_base 1421: mod_base = OTHER; note = Uracil
  modified_base 1426: mod_base = OTHER; note = Uracil
  modified_base 1429: mod_base = OTHER; note = Uracil
  modified_base 1444: mod_base = OTHER; note = Uracil
  modified_base 1445: mod_base = OTHER; note = Uracil
  modified_base 1448: mod_base = OTHER; note = Uracil
  modified_base 1454: mod_base = OTHER; note = Uracil
  modified_base 1460: mod_base = OTHER; note = Uracil
  modified_base 1467: mod_base = OTHER; note = Uracil
  modified_base 1476: mod_base = OTHER; note = Uracil
  modified_base 1477: mod_base = OTHER; note = Uracil
  modified_base 1481: mod_base = OTHER; note = Uracil
  modified_base 1482: mod_base = OTHER; note = Uracil
  modified_base 1486: mod_base = OTHER; note = Uracil
  modified_base 1488: mod_base = OTHER; note = Uracil
  modified_base 1492: mod_base = OTHER; note = Uracil
  modified_base 1496: mod_base = OTHER; note = Uracil
  modified_base 1503: mod_base = OTHER; note = Uracil
  modified_base 1505: mod_base = OTHER; note = Uracil
  modified_base 1509: mod_base = OTHER; note = Uracil
  modified_base 1511: mod_base = OTHER; note = Uracil
  modified_base 1514: mod_base = OTHER; note = Uracil
  modified_base 1516: mod_base = OTHER; note = Uracil
  modified_base 1517: mod_base = OTHER; note = Uracil
  modified_base 1520: mod_base = OTHER; note = Uracil
  modified_base 1528: mod_base = OTHER; note = Uracil
  modified_base 1535: mod_base = OTHER; note = Uracil
  modified_base 1541: mod_base = OTHER; note = Uracil
  modified_base 1543: mod_base = OTHER; note = Uracil
  modified_base 1545: mod_base = OTHER; note = Uracil
  modified_base 1546: mod_base = OTHER; note = Uracil
  modified_base 1551: mod_base = OTHER; note = Uracil
  modified_base 1559: mod_base = OTHER; note = Uracil
  modified_base 1567: mod_base = OTHER; note = Uracil
  modified_base 1568: mod_base = OTHER; note = Uracil
  modified_base 1570: mod_base = OTHER; note = Uracil
  modified_base 1571: mod_base = OTHER; note = Uracil
  modified_base 1575: mod_base = OTHER; note = Uracil
  modified_base 1576: mod_base = OTHER; note = Uracil
  modified_base 1578: mod_base = OTHER; note = Uracil
  modified_base 1584: mod_base = OTHER; note = Uracil
  modified_base 1593: mod_base = OTHER; note = Uracil
  modified_base 1598: mod_base = OTHER; note = Uracil
  modified_base 1604: mod_base = OTHER; note = Uracil
  modified_base 1605: mod_base = OTHER; note = Uracil
  modified_base 1606: mod_base = OTHER; note = Uracil
  modified_base 1613: mod_base = OTHER; note = Uracil
  modified_base 1618: mod_base = OTHER; note = Uracil
  modified_base 1619: mod_base = OTHER; note = Uracil
  modified_base 1621: mod_base = OTHER; note = Uracil
  modified_base 1623: mod_base = OTHER; note = Uracil
  modified_base 1632: mod_base = OTHER; note = Uracil
  modified_base 1639: mod_base = OTHER; note = Uracil
  modified_base 1646: mod_base = OTHER; note = Uracil
  modified_base 1658: mod_base = OTHER; note = Uracil
  modified_base 1659: mod_base = OTHER; note = Uracil
  modified_base 1676: mod_base = OTHER; note = Uracil
  modified_base 1679: mod_base = OTHER; note = Uracil
  modified_base 1681: mod_base = OTHER; note = Uracil
  modified_base 1682: mod_base = OTHER; note = Uracil
  modified_base 1683: mod_base = OTHER; note = Uracil
  modified_base 1691: mod_base = OTHER; note = Uracil
  modified_base 1708: mod_base = OTHER; note = Uracil
  modified_base 1714: mod_base = OTHER; note = Uracil
  modified_base 1715: mod_base = OTHER; note = Uracil
  modified_base 1721: mod_base = OTHER; note = Uracil
  modified_base 1737: mod_base = OTHER; note = Uracil
  modified_base 1744: mod_base = OTHER; note = Uracil
  modified_base 1753: mod_base = OTHER; note = Uracil
  modified_base 1758: mod_base = OTHER; note = Uracil
  modified_base 1764: mod_base = OTHER; note = Uracil
  modified_base 1766: mod_base = OTHER; note = Uracil
  modified_base 1767: mod_base = OTHER; note = Uracil
  modified_base 1768: mod_base = OTHER; note = Uracil
  modified_base 1786: mod_base = OTHER; note = Uracil
  modified_base 1787: mod_base = OTHER; note = Uracil
  modified_base 1790: mod_base = OTHER; note = Uracil
  modified_base 1800: mod_base = OTHER; note = Uracil
  modified_base 1820: mod_base = OTHER; note = Uracil
  modified_base 1838: mod_base = OTHER; note = Uracil
  modified_base 1839: mod_base = OTHER; note = Uracil
  modified_base 1845: mod_base = OTHER; note = Uracil
  modified_base 1848: mod_base = OTHER; note = Uracil
  modified_base 1855: mod_base = OTHER; note = Uracil
  modified_base 1859: mod_base = OTHER; note = Uracil
  modified_base 1863: mod_base = OTHER; note = Uracil
  modified_base 1877: mod_base = OTHER; note = Uracil
  modified_base 1883: mod_base = OTHER; note = Uracil
  modified_base 1889: mod_base = OTHER; note = Uracil
  modified_base 1892: mod_base = OTHER; note = Uracil
  modified_base 1899: mod_base = OTHER; note = Uracil
  modified_base 1907: mod_base = OTHER; note = Uracil
  modified_base 1913: mod_base = OTHER; note = Uracil
  modified_base 1916: mod_base = OTHER; note = Uracil
  modified_base 1919: mod_base = OTHER; note = Uracil
  modified_base 1922: mod_base = OTHER; note = Uracil
  modified_base 1924: mod_base = OTHER; note = Uracil
  modified_base 1925: mod_base = OTHER; note = Uracil
  modified_base 1928: mod_base = OTHER; note = Uracil
  modified_base 1931: mod_base = OTHER; note = Uracil
  modified_base 1932: mod_base = OTHER; note = Uracil
  modified_base 1934: mod_base = OTHER; note = Uracil
  modified_base 1937: mod_base = OTHER; note = Uracil
  modified_base 1940: mod_base = OTHER; note = Uracil
  modified_base 1943: mod_base = OTHER; note = Uracil
  modified_base 1949: mod_base = OTHER; note = Uracil
  modified_base 1951: mod_base = OTHER; note = Uracil
  modified_base 1952: mod_base = OTHER; note = Uracil
  modified_base 1954: mod_base = OTHER; note = Uracil
  modified_base 1955: mod_base = OTHER; note = Uracil
  modified_base 1957: mod_base = OTHER; note = Uracil
  modified_base 1958: mod_base = OTHER; note = Uracil
  modified_base 1972: mod_base = OTHER; note = Uracil
  modified_base 1976: mod_base = OTHER; note = Uracil
  modified_base 1978: mod_base = OTHER; note = Uracil
  modified_base 1986: mod_base = OTHER; note = Uracil
  modified_base 1990: mod_base = OTHER; note = Uracil
  modified_base 1993: mod_base = OTHER; note = Uracil
  modified_base 1994: mod_base = OTHER; note = Uracil
  modified_base 1997: mod_base = OTHER; note = Uracil
  modified_base 2001: mod_base = OTHER; note = Uracil
  modified_base 2003: mod_base = OTHER; note = Uracil
  modified_base 2009: mod_base = OTHER; note = Uracil
  modified_base 2012: mod_base = OTHER; note = Uracil
  modified_base 2016: mod_base = OTHER; note = Uracil
  modified_base 2018: mod_base = OTHER; note = Uracil
  modified_base 2021: mod_base = OTHER; note = Uracil
  modified_base 2024: mod_base = OTHER; note = Uracil
  modified_base 2025: mod_base = OTHER; note = Uracil
  modified_base 2026: mod_base = OTHER; note = Uracil
  modified_base 2030: mod_base = OTHER; note = Uracil
  modified_base 2031: mod_base = OTHER; note = Uracil
  modified_base 2033: mod_base = OTHER; note = Uracil
  modified_base 2034: mod_base = OTHER; note = Uracil
  modified_base 2037: mod_base = OTHER; note = Uracil
  modified_base 2038: mod_base = OTHER; note = Uracil
  modified_base 2041: mod_base = OTHER; note = Uracil
  modified_base 2042: mod_base = OTHER; note = Uracil
  modified_base 2046: mod_base = OTHER; note = Uracil
  modified_base 2047: mod_base = OTHER; note = Uracil
atgataagaggccgcgccccacgcacccgccctagtccccctcctcctttgttgccacttctttctctgtcccttctcctcctctctcctactgtgaggggtcacaagtatcaccaacaccactgcgacgacagcagccatcaccaccatcggcacaaccaccatcatcacctcaaccgtcaccaccgatgctgtctcgccgcggtgtttctgctgctggccgtcgcgatcgtcagccccggacgagccgaacacgttgtacttttggacaccacgacagagcctagtctacggtggactacgtacccttacggaccggacgccaacgcagcagggtgggtcgaagagagctacataaattttgaaaagggcatcaactggcggtcgtacgtcgtgtgcgatgtcaccaagcaaaacgtcaacaattgggtgtggacgcctttcatagagcgcgggctggccaatctcatttacatcgaagtcaagtttaccatccgagattgttcgctgttcccggggtacgcgctgtcatgcaaagaaacgttttcactactctactatgagtttgacgtggccaccagggaaccaccgccttgggaacctgacagctataaatctgttggtcggatagctgctggtgaagggaggttcaacgctaataacgaagtggtgatcaatactgagacaaaagctgtgaaagttaccaaaaaaggagtgtactttgccttcagggaccagggcgcatgtatttccatcatggcagtcaaggtgtattacatcgtgtgcccagaagttgtgataaactttgcaaacttctctgctactccaactgctagagaacttacccaaatcgaacatgccaccggcaaatgtgttgataatgctgaagtggtaggaggtggtgctccaacttacctgtgcaaaggagatggaaaatggtatctaccgtcaggtggatgcaagtgcaaagctggttttgaagcagacatagaagcgcaaacttgtataatctgtcctccgggcaagtacaaatacggcgttggggacgacaagtgtcagccgtgtccggcgcacagcaaagcgcccgaccaggggatgtccgagtgcaggtgcaacaccggatactacaggtcgcccaaggacccaaaatcggtgccgtgcacgcaaccgccgtcagcgccgcaaaacttgacagtcaactttgtggaccagtcgactgtgaccctgtcgtggaacccaccgaacttcctcggtggcaggaccgatatcgtgtacagagtgacatgcgacatgtgcggtccatccgttgtgttcatgcccaacaacgaggtgttcaacgataccaaaataacgatcagcggtctgggtccggtcaccacgtacaagtttcacgtatgggccgaaaacggtgtcagtaatctaacgtcgtccgaaaaccggcaattcgtcgacatagccgtgaccactacagaagcttccgttaagtctgcgtccgtcaacaatgtccgtgtcatgttggtaaaggcgtccgagatcacgctatcttgggatccgcccatggcgagcttcttcgattctggcgatgacgacgctgccgtcgaagtttacgaagtgaagttctatccccgaggtgacgagtcaaacgtcagcaacaaacttaccgccgacaggcacatggtgtttacggcactcaggcccaagacggactacggattccaagtgcgagcgaagaccgctcacggctggggcgagtacagtccgactatttacaaaacgacggggcaattgctcggaagcggtaagaacacacaacaacaagtccaaagaaaaaacgggattaaagatgatacggcctacatcggtgacgaagacaacatggaagtccgaataatcgcaggtgcgacggtcgcggtagtggtggtcttagtagttgtcatcatcatgacggtattgttcttaggaggagaccggtacatatacgggcatacctgcttgataactctgacagtgctgcatgtgatgctttcacttattggttacttgacttga